SEQUENCE LISTING (94 sequences)

SEQ ID NO: 1 - protein, 8 aa, mus musculus
  misc_feature: Antibody 3E2-Heavy chain-CDR1
GFDFSRYW

SEQ ID NO: 2 - protein, 8 aa, mus musculus
  misc_feature: Antibody 3E2-Heavy chain-CDR2
INPDSSTI

SEQ ID NO: 3 - protein, 12 aa, mus musculus
  misc_feature: Antibody3E2-Heavy chain-CDR3
ARGDYYGSRCDY

SEQ ID NO: 4 - protein, 6 aa, mus musculus
  misc_feature: Antibody 3E2-Light chain-CDR1
QDIKSY

SEQ ID NO: 5 - protein, 3 aa, mus musculus
  misc_feature: Antibody 3E2-Light chain-CDR2
YAT

SEQ ID NO: 6 - protein, 9 aa, mus musculus
  misc_feature: Antibody 3E2-Light chain-CDR3
LQHGESPLT

SEQ ID NO: 7 - protein, 8 aa, mus musculus
  misc_feature: Antibody 14C11-Heavy chain-CDR1
GFSLTGYG

SEQ ID NO: 8 - protein, 7 aa, mus musculus
  misc_feature: Antibody 14C11-Heavy chain-CDR2
IWGDGST

SEQ ID NO: 9 - protein, 11 aa, mus musculus
  misc_feature: Antibody 14C11-Heavy chain-CDR3
ARNGNYLAFDY

SEQ ID NO: 10 - protein, 6 aa, mus musculus
  misc_feature: Antibody 14C11-Light chain-CDR1
QSISDY

SEQ ID NO: 11 - protein, 3 aa, mus musculus
  misc_feature: Antibody 14C11-Light chain-CDR2
YAS

SEQ ID NO: 12 - protein, 9 aa, mus musculus
  misc_feature: Antibody 14C11-Light chain-CDR3
QNGHSFPLT

SEQ ID NO: 13 - protein, 8 aa, mus musculus
  misc_feature: Antibody 15C11-Heavy chain-CDR1
GYAFSSSW

SEQ ID NO: 14 - protein, 8 aa, mus musculus
  misc_feature: Antibody 15C11-Heavy chain-CDR2
IYPGDGDT

SEQ ID NO: 15 - protein, 10 aa, mus musculus
  misc_feature: Antibody 15C11-Heavy chain-CDR3
ARGDRAWFAY

SEQ ID NO: 16 - protein, 7 aa, mus musculus
  misc_feature: Antibody 15C11-Light chain-CDR1
SSISSNY

SEQ ID NO: 17 - protein, 3 aa, mus musculus
  misc_feature: Antibody 15C11-Light chain-CDR2
RTS

SEQ ID NO: 18 - protein, 9 aa, mus musculus
  misc_feature: Antibody 15C11-Light chain-CDR3
QQGSSIPRT

SEQ ID NO: 19 - protein, 8 aa, mus musculus
  misc_feature: Antibody 22F6-Heavy chain-CDR1
GYTFTSYW

SEQ ID NO: 20 - protein, 8 aa, mus musculus
  misc_feature: Antibody 22F6-Heavy chain-CDR2
IYPGSGST

SEQ ID NO: 21 - protein, 8 aa, mus musculus
  misc_feature: Antibody 22F6-Heavy chain-CDR3
TRKFLFDY

SEQ ID NO: 22 - protein, 6 aa, mus musculus
  misc_feature: Antibody 22F6-Light chain-CDR1
QSISNY

SEQ ID NO: 23 - protein, 3 aa, mus musculus
  misc_feature: Antibody 22F6-Light chain-CDR2
YAS

SEQ ID NO: 24 - protein, 9 aa, mus musculus
  misc_feature: Antibody 22F6-Light chain-CDR3
QQSNSWPHT

SEQ ID NO: 25 - protein, 8 aa, mus musculus
  misc_feature: Antibody 25C10-Heavy chain-CDR1
GYSFTSYW

SEQ ID NO: 26 - protein, 8 aa, mus musculus
  misc_feature: Antibody 25C10-Heavy chain-CDR2
IHPSDSET

SEQ ID NO: 27 - protein, 8 aa, mus musculus
  misc_feature: Antibody 25C10-Heavy chain-CDR3
AGGDRYGY

SEQ ID NO: 28 - protein, 10 aa, mus musculus
  misc_feature: Antibody 25C10-Light chain-CDR1
ESVDNYGISF

SEQ ID NO: 29 - protein, 3 aa, mus musculus
  misc_feature: Antibody 25C10-Light chain-CDR2
AAS

SEQ ID NO: 30 - protein, 9 aa, mus musculus
  misc_feature: Antibody 25C10-Light chain-CDR3
QQSKEVPRT

SEQ ID NO: 31 - protein, 8 aa, mus musculus
  misc_feature: Antibody 11E10-Heavy chain-CDR1
GYTFTNYG

SEQ ID NO: 32 - protein, 8 aa, mus musculus
  misc_feature: Antibody 11E10-Heavy chain-CDR2
INTYTGEP

SEQ ID NO: 33 - protein, 5 aa, mus musculus
  misc_feature: Antibody 11E10-Heavy chain-CDR3
ASGVY

SEQ ID NO: 34 - protein, 11 aa, mus musculus
  misc_feature: Antibody 11E10-Light chain-CDR1
QSLLYSNGKTY

SEQ ID NO: 35 - protein, 3 aa, mus musculus
  misc_feature: Antibody 11E10-Light chain-CDR2
LVS

SEQ ID NO: 36 - protein, 8 aa, mus musculus
  misc_feature: Antibody 11E10-Light chain-CDR3
VQGTHLDV

SEQ ID NO: 37 - protein, 8 aa, mus musculus
  misc_feature: Antibody 16G8-Heavy chain-CDR1
GFTFSDYY

SEQ ID NO: 38 - protein, 8 aa, mus musculus
  misc_feature: Antibody 16G8-Heavy chain-CDR2
ISNGGGST

SEQ ID NO: 39 - protein, 10 aa, mus musculus
  misc_feature: Antibody 16G8-Heavy chain-CDR3
ARPTYGYFDY

SEQ ID NO: 40 - protein, 5 aa, mus musculus
  misc_feature: Antibody 16G8-Light chain-CDR1
SSVSY

SEQ ID NO: 41 - protein, 3 aa, mus musculus
  misc_feature: Antibody 16G8-Light chain-CDR2
ATS

SEQ ID NO: 42 - protein, 9 aa, mus musculus
  misc_feature: Antibody 16G8-Light chain-CDR3
QQWSSNPPT

SEQ ID NO: 43 - protein, 101 aa, mus musculus
  misc_feature: Antibody 3E2-Heavy chain-Variable region
ELSCAASGFDFSRYWMSWVRQAPGKGLEWIGEINPDSSTINYTPSLKDKFIISRDNAKNTLYLQMSKVRSEDTALYYCARGDYYGSRCDYWGQGTTLQSPQ

SEQ ID NO: 44 - protein, 101 aa, mus musculus
  misc_feature: Antibody 14C11-Heavy chain-Variable region
SLSITCTVSGFSLTGYGVNWVRQPPGKGLEWLGMIWGDGSTDYNSALKSRLSISKDNSKSQVFLKMNSLQTDDTARYYCARNGNYLAFDYWGQGTTLQSSS

SEQ ID NO: 45 - protein, 90 aa, mus musculus
  misc_feature: Antibody 15C11-Heavy chain-Variable region
ASGYAFSSSWMNWVKQRPGQGLEWIGRIYPGDGDTNYNGKFKGKATLTADKSSSTAYMQLSSLTSVDSAVYFCARGDRAWFAYWGQGTLV

SEQ ID NO: 46 - protein, 95 aa, mus musculus
  misc_feature: Antibody 22F6-Heavy chain-Variable region
LSCKASGYTFTSYWMHWVKQRPGQGLEWIGNIYPGSGSTNYDEKFKSKATLTVDTSSSTAYMQLSSLTSEDSAVYYCTRKFLFDYWGQGTTLTVS

SEQ ID NO: 47 - protein, 95 aa, mus musculus
  misc_feature: Antibody 25C10-Heavy chain-Variable region
LSCKASGYSFTSYWMNWVKQRPGQGLEWIGMIHPSDSETRLNQKFKDKATLTVDKSSSTAYMQLSSPTSEDSAVYYCAGGDRYGYWGQGTTLTVS

SEQ ID NO: 48 - protein, 94 aa, mus musculus
  misc_feature: Antibody 11E10-Heavy chain-Variable region
QISCKASGYTFTNYGMNWVKQAPGKGLKWMGWINTYTGEPTYADDFKGRFAFSLETSASTAYLQINNLKNEDTATYFCASGVYWGQGTTLTVSS

SEQ ID NO: 49 - protein, 100 aa, mus musculus
  misc_feature: Antibody 16G8-Heavy chain-Variable region
PELSCATSGFTFSDYYMYWVRQTPEKRLEWVAYISNGGGSTYYPDTVKGRFTISRDNAKNTLYLQMSRLKSEDTAMYYCARPTYGYFDYWGQGTTLTVSS

SEQ ID NO: 50 - protein, 88 aa, mus musculus
  misc_feature: Antibody 3E2-Light chain-Variable region
ITCKASQDIKSYLSWYQQKPWKSPKTLIYYATSLADGVPSRFSGSGSGQDYSLTISSLESDDTATYYCLQHGESPLTFGAGTKLELKR

SEQ ID NO: 51 - protein, 89 aa, mus musculus
  misc_feature: Antibody 14C11-Light chain-Variable region
SLSCRASQSISDYLHWYQQKSHESPRLLIKYASQSISGIPSRFSGSGSGSDFTLSINSVEPEDVGVYYCQNGHSFPLTFGAGTKLELKR

SEQ ID NO: 52 - protein, 85 aa, mus musculus
  misc_feature: Antibody 15C11-Light chain-Variable region
XTCSASSSISSNYLHWYQQKPGFSPKLLIYRTSNLASGVPARFSGSGSGTSYSLTIGTMEAEDVATYYCQQGSSIPRTFGGGTKL

SEQ ID NO: 53 - protein, 88 aa, mus musculus
  misc_feature: Antibody 22F6-Light chain-Variable region
LSCRASQSISNYLHWYQQKSHESPRLLIKYASQSISGIPSRFSGSGSGTDFTLSINSVETENFGMYFCQQSNSWPHTFGAGTKLELKR

SEQ ID NO: 54 - protein, 88 aa, mus musculus
  misc_feature: Antibody 25C10-Light chain-Variable region
ISCRASESVDNYGISFMNWFQQKPGQPPKLLIYAASNQGSGVPARFSGSGSGTDFSLNIHPMEEDDTAMYFCQQSKEVPRTFGGGTKL

SEQ ID NO: 55 - protein, 87 aa, mus musculus
  misc_feature: Antibody 11E10-Light chain-Variable region
ISCKSSQSLLYSNGKTYLNWLLQRPGQSPKRLIYLVSKLDSGVPDRFTGSGSGTDFTLKISRVEAEDLGVYYCVQGTHLDVRWRHQA

SEQ ID NO: 56 - protein, 82 aa, mus musculus
  misc_feature: Antibody 16G8-Light chain-Variable region
TCRASSSVSYMHWYQQKPGSSPKPWIYATSNLASGVPARFSGSGSGTSYSLTISKVEAEDAATYYCQQWSSNPPTFGAGTKL

SEQ ID NO: 57 - DNA, 304 bp, mus musculus
  misc_feature: Antibody 3E2-Heavy chain-Variable region
tgaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagaggggattactacggtagtagatgtgactactggggccaaggcaccactctccagtctcctcaa

SEQ ID NO: 58 - DNA, 305 bp, mus musculus
  misc_feature: Antibody 14C11-Heavy chain-Variable region
gagcctgtccatcacatgcaccgtctcagggttctcattaaccggctatggtgtaaactgggttcgccagcctccaggaaagggtctggagtggctgggaatgatatggggtgatggaagcacagactataattcagctctcaaatccagactgagcatcagcaaggacaactccaagagccaagttttcttaaaaatgaacagtctgcaaactgatgacacagccaggtactactgtgccagaaatggtaactacctcgcctttgactactggggccaaggcaccactctccagtcctcctcaa

SEQ ID NO: 59 - DNA, 272 bp, mus musculus
aagcttctggctacgcattcagtagctcttggatgaactgggtgaagcagaggcctggacagggtcttgagtggattggacggatttatcctggagatggagatactaactacaatgggaagttcaagggcaaggccacactgactgcagacaaatcctccagcacagcctacatgcagctcagcagcctgacctctgtggactctgcggtctatttctgtgcaagaggggacagggcctggtttgcttactggggccaagggactctggtc

SEQ ID NO: 60 - DNA, 287 bp, mus musculus
  misc_feature: Antibody 22F6-Heavy chain-Variable region
gctgtcctgcaaggcttctggctacacattcaccagctactggatgcactgggtgaagcagaggcctggacaaggccttgagtggattggaaatatttatcctggtagtggtagtactaactacgatgagaagttcaagagcaaggccacactgactgtagacacatcctccagcacagcctacatgcagctcagcagcctgacatctgaggactctgcggtctattactgtacaagaaagttcctctttgactactggggccaaggcaccactctcacagtctcct

SEQ ID NO: 61 - DNA, 288 bp, mus musculus
  misc_feature: Antibody 25C10-Heavy chain-Variable region
gctgtcctgcaaggcttctggctactccttcaccagctactggatgaactgggtgaagcagaggcctggacaaggccttgagtggattggcatgattcatccttccgatagtgaaactaggttaaatcagaagttcaaggacaaggccacattgactgtagacaaatcctccagcacagcctacatgcaactcagcagcccgacatctgaggactctgcggtctattactgtgcagggggggataggtacggctactggggccaaggcaccactctcacagtctcctc

SEQ ID NO: 62 - DNA, 285 bp, mus musculus
  misc_feature: Antibody 11E10-Heavy chain-Variable region
gtcagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacacggctacatatttctgtgcaagtggggtttactggggccaaggcaccactctcacagtctcctcaa

SEQ ID NO: 63 - DNA, 301 bp, mus musculus
  misc_feature: Antibody 16G8-Heavy chain-Variable region
cctgaactctcctgtgcaacctctggattcactttcagtgactattacatgtattgggttcgccagactccagagaagaggctggagtgggtcgcatacattagtaatggtggtggtagcacctattatccagacactgtaaagggccgattcaccatctccagagacaatgccaagaacaccctgtacctgcaaatgagccgtctgaagtctgaggacacagccatgtattactgtgcaagaccgacgtatggttactttgactactggggccaaggcaccactctcacagtctcctcaa

SEQ ID NO: 64 - DNA, 265 bp, mus musculus
  misc_feature: Antibody 3E2-Light chain-Variable region
tatcacttgcaaggcgagtcaggacattaaaagctatttaagctggtaccagcagaaaccatggaaatctcctaagaccctgatctattatgcaacaagcttggcagatggggtcccatcaagattcagtggcagtggatctgggcaagattattctctaaccatcagcagcctggagtctgacgatacagcaacttattactgtctacagcatggtgagagcccgctcacgttcggtgctgggaccaagctggagctgaaacgg

SEQ ID NO: 65 - DNA, 267 bp, mus musculus
  misc_feature: Antibody 14C11-Light chain-Variable region
tctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggtgctgggaccaagctggagctgaaacgg

SEQ ID NO: 66 - DNA, 254 bp, mus musculus
  misc_feature: Antibody 15C11-Light chain-Variable region
tcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccacgcacgttcggaggggggaccaagctg

SEQ ID NO: 67 - DNA, 266 bp, mus musculus
  misc_feature: Antibody 22F6-Light chain-Variable region
gtctttcctgcagggccagtcaaagtattagcaactacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaaaattttggaatgtatttctgtcaacagagtaacagctggcctcacacgttcggtgctgggaccaagctggagctgaaacgg

SEQ ID NO: 68 - DNA, 266 bp, mus musculus
  misc_feature: Antibody 25C10-Light chain-Variable region
ccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttcctcggacgttcggtggaggcaccaagctg

SEQ ID NO: 69 - DNA, 263 bp, mus musculus
  misc_feature: Antibody 11E10-Light chain-Variable region
atctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacatttggacgttcggtggaggcaccaagctgg

SEQ ID NO: 70 - DNA, 250 bp, mus musculus
  misc_feature: Antibody 16G8-Light chain-Variable region
tgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcaaagtggaggctgaagatgctgccacttattactgccagcagtggagtagtaacccacccacgttcggtgctgggaccaagctgga

SEQ ID NO: 71 - DNA, 22 bp, artificial
  Back PCR primer VH1 A
aggtgcagcttcaggagtcagg

SEQ ID NO: 72 - DNA, 22 bp, artificial
  Back PCR primer VH1 B
aggtgcagctagaggagtcagg

SEQ ID NO: 73 - DNA, 22 bp, artificial
  Back PCR primer VH2 A
aggtccagctgcagcagtcatg

SEQ ID NO: 74 - DNA, 22 bp, artificial
  Back PCR primer VH2 B
aggtccacctgcagcagcctgg

SEQ ID NO: 75 - DNA, 22 bp, artificial
  Back PCR primer VH2 C
aggtccagctgcagcagtctgg

SEQ ID NO: 76 - DNA, 22 bp, artificial
  Back PCR primer VH3 A
aggtgaagctggtggagtctgg

SEQ ID NO: 77 - DNA, 22 bp, artificial
  Back PCR primer VH3 B
aggtgaagcttctcgagtctgg

SEQ ID NO: 78 - DNA, 22 bp, artificial
  Back PCR primer VH3 C
aagtgaagcttgaggagtctgg

SEQ ID NO: 79 - DNA, 22 bp, artificial
  Back PCR primer VH3 D
aagtgatgctggtggagtcgtg

SEQ ID NO: 80 - DNA, 22 bp, artificial
  Back PCR primer VH5
aggttcagctccagcagtctgg

SEQ ID NO: 81 - DNA, 24 bp, artificial
  Forward PCR primer JH1
tgaggagacggtgaccgtggtccc

SEQ ID NO: 82 - DNA, 24 bp, artificial
  Forward PCR primer JH2
tgaggagactgtgagagtggtgcc

SEQ ID NO: 83 - DNA, 24 bp, artificial
  Forward PCR primer JH3
tgcagagacagtgaccagagtccc

SEQ ID NO: 84 - DNA, 24 bp, artificial
  Forward PCR primer JH4
tgaggagacggtgactgaggttcc

SEQ ID NO: 85 - DNA, 24 bp, artificial
  Back PCR primer VkA
gatgttttgatgacccaaactcca

SEQ ID NO: 86 - DNA, 24 bp, artificial
  Back PCR primer VkB
gatattgtgataacccaggatgaa

SEQ ID NO: 87 - DNA, 24 bp, artificial
  Back PCR primer VkC
gacattgtgctracccartctcca

SEQ ID NO: 88 - DNA, 24 bp, artificial
  Back PCR primer VkD
gacatccagatgacncagtctcca

SEQ ID NO: 89 - DNA, 24 bp, artificial
  Back PCR primer VkE
caaattgttctcacccagtctcca

SEQ ID NO: 90 - DNA, 24 bp, artificial
  Back PCR primer VkF
gaaaatgtgctcacccagtctcca

SEQ ID NO: 91 - DNA, 21 bp, artificial
  Forward PCR primer Jk1
ccgtttgatcagcttggtgcc

SEQ ID NO: 92 - DNA, 24 bp, artificial
  Forward PCR primer Jk2
ccgttttatttccagcttggtccc

SEQ ID NO: 93 - DNA, 24 bp, artificial
  Forward PCR primer Jk3
ccgttttatttccaactttgtccc

SEQ ID NO: 94 - DNA, 24 bp, artificial
  Forward PCR primer Jk4
ccgtttcagctccagcttggtccc

(19) United States
(12) Patent Application Publication
(10) Pub. No.: US 2014/0044721 A1
(43) Pub. Date: Feb. 13, 2014

(54) ANTI-GB3 ANTIBODIES USEFUL IN TREATING DISORDERS ASSOCIATED WITH ANGIOGENESIS

(71) Applicants: François Paris, Nantes (FR); Stephane Birkle, Nantes (FR); Ariane Desselle, Montpellier (FR); Jacques Aubry, Nantes (FR); Marie-Hélène Gaugler, Nantes (FR); Denis Cochonneau, Laigne En Belin (FR); Tanguy Chaumette, Nantes (FR)

(72) Inventors: François Paris, Nantes (FR); Stephane Birkle, Nantes (FR); Ariane Desselle, Montpellier (FR); Jacques Aubry, Nantes (FR); Marie-Hélène Gaugler, Nantes (FR); Denis Cochonneau, Laigne En Belin (FR); Tanguy Chaumette, Nantes (FR)

(73) Assignees: INSTITUT DE RADIOPROTECTION ET DE SURETE NUCLEAIRE, Fontenay-Aux-Roses (FR); UNIVERSITE DE NANTES, Nantes (FR)

(21) Appl. No.: 13/983,937

(22) PCT Filed: Feb. 7, 2012
(86) PCT No.: PCT/EP2012/052004

§ 371 Date: Oct. 21, 2013

(30) Foreign Application Priority Data
Feb. 7, 2011 (FR) 1150958

(51) Int. Cl. C07K 16/28

(52) U.S. Cl. CPC C07K 16/28
USPC 424/137.1; 530/387.5; 530/387.3; 536/23.53; 435/320.1; 435/329

(57) ABSTRACT

The present invention lies in the field of new cancer therapies, more precisely in the field of antiangiogenic compounds. It notably concerns anti-Gb3 antibodies having specific CDR sequences, as well as the use of anti-Gb3 antibodies not coupled to a therapeutic molecule in the treatment of diseases associated with angiogenesis, such as solid tumors.

### FIELD OF THE INVENTION

The present invention lies in the field of new cancer therapies, more precisely in the field of antiangiogenic compounds. It notably concerns anti-Gb3 antibodies having specific CDR sequences, as well as the use of anti-Gb3 antibodies not coupled to a therapeutic molecule in the treatment of diseases associated with angiogenesis.

### PRIOR ART

Cancer cells, as a result of their genetic instability, may acquire resistance to cancer treatments, which causes treatment failure or cancer relapse.

There is therefore a need for new anticancer agents less sensitive to the genetic variability of tumor cells.

Unlike tumor cells themselves, endothelial cells of tumor blood vessels are stable genetically. Moreover, they are necessary to neovascularization, without which the tumor cannot continue to grow, due to lack of nutrients. Consequently, a strategy seeking to inhibit angiogenesis, i.e., the proliferation of tumor endothelial cells, would not be sensitive to tumor genetic variability, since endothelial cells are genetically stable.

Targeting endothelial cells also has other advantages:
In the case of a solid tumor, tumor cells are difficult to reach. With traditional cancer therapies, the penetration of treating agents into tumor tissues is reduced due to the high interstitial pressure in most tumors. This is not the case in the vessels, which constitute a more easily accessible target. As a result of this accessibility, targeting endothelial cells allows effective distribution and fast accumulation in tumor vessels. As a result, targeting vascularization is a strategy that can be applied to the majority of tumor types.
In an adult organism, the vast majority of endothelial cells are in a quiescent state. In return, the endothelium of tumor vessels is composed of endothelial cells with a so-called “angiogenic” proliferating phenotype, which it is theoretically possible to target. Indeed, the vessels that supply tumors have different structural and functional characteristics than those of normal vessels. Therefore, this strategy is expected to be selective and induce few side effects on so-called “physiological” angiogenesis.
Finally, targeting vascularization and inhibiting the formation of metastases are key aspects, since invasion is one of the most serious aspects of the disease.

Thus, targeting proliferating endothelial cells has many advantages, in terms of accessibility of the target cells, the number of tumors that can be treated, the reduction of side effects if only these proliferating endothelial cells are targeted, and the prevention of metastases.

Globotriaosylceramide (Gb3), also called CD77, P^{k}, ceramide trihexoside (CTH) and Burkitt lymphoma antigen (BLA) is a neutral glycosphingolipid of formula galactose α1→4 galactose β1→4 glucose β1→ ceramide, synthesized by the enzyme lactosylceramide 4-alpha-galactosyltransferase (A4GALT). It is expressed by HUVEC endothelial cell line, more when this line is proliferating than when the cells are confluent and therefore no longer in the exponential growth phase. It is therefore an endothelial cell marker involved in angiogenesis (Heath-Engel et al., Obrig et al.).

Ceramide, the molecule hydrophobic part anchored in the outer layer of the plasma membrane, is formed of a fatty acid chain composed of 16 to 28 carbon atoms, bound by an amide bond to a sphingoid base, typically sphingosine. It may have variations in the chain length and the number of unsaturated carbons of the fatty acid chain. Notably, the main variations possible for the sphingoid base and the fatty acid chain are shown in Table 1 below.

TABLE 1
Main molecular types of fatty acids
and sphingoid bases used in ceramide.

| Fatty acids | Sphingoid bases |
|---|---|
| Palmitic acid (16:0) | Sphingosine (18:1) |
| Stearic acid (18:0) | Dihydrosphingosine (18:0) |
| Oleic acid (18:1) | C20-dihydrosphingosine (20:0) |
| Arachidonic acid (20:0) | C20-sphingosine (20:1) |
| Behenic acid (22:0) | |
| Lignoceric acid (24:0) | |
| Nervonic acid (24:1) | |

Gb3 is a natural receptor for certain bacterial toxins such as Shiga toxin, produced by Shigella dysenteriae, and verotoxins produced by Escherichia coli. These toxins are composed of two subunits A and B, subunit B being involved in Gb3 binding, while subunit A corresponds to the toxic part inhibiting protein synthesis. When these bacterial toxins bind Gb3, they are transported from the plasma membrane to the endoplasmic reticulum via the early endosomes and Golgi apparatus. Subunit A produces its toxic effects in the endoplasmic reticulum.

Shiga toxin and verotoxin have therefore been proposed to exert a cytotoxic activity on endothelial cells, in particular on proliferating endothelial cells (Heath-Engel et al., Obrig et al. WO98/51326).

Verotoxins and Shiga toxin have also been proposed as a therapeutic agent in the treatment of tumors expressing Gb3 on their surface, subunit B serving for targeting while subunit A plays a cytotoxic role. In particular, verotoxin 1 has been shown to be able to induce apoptosis in Burkitt lymphoma cells (Tétaud et al.).

However, these approaches are not really applicable in cancer therapy due to the very substantial side effects of the toxic subunit A on the rest of the human body. Indeed, in addition to vascular tissue, Gb3 is expressed in many other tissues, such as stomach, esophagus, prostate and kidney tissue. Now, since Shiga toxin and verotoxins are small molecules (around 68 kilodaltons (KDa)), they can leave the bloodstream and enter healthy tissue, where they have the same cytotoxic properties for cells expressing Gb3 as for tumor cells. Due to their lack of specificity of action, these toxins lead to unacceptable side effects. Thus, for example, Bast et al. show that two hours after administration to rabbits, verotoxin 1 is no longer present in the bloodstream and can be found in target organs expressing Gb3.

Moreover, in addition to the associated side effects, the small molecular mass of these toxins and the fact that they leave the bloodstream also reduces their duration of action on blood vessel cells, thus also reducing their efficacy.

Using only subunit B of these toxins has also been proposed, notably for Shiga toxin, to target tumor cells expressing Gb3, with a cytotoxic therapeutic molecule coupled to subunit B (Janssen et al. Viel et al.). However, as long as the therapeutic molecule coupled to subunit B (only about 8 kDa per subunit B) has a low molecular weight, then this strategy poses the same problems of toxicity and duration of action on blood vessel cells as the previous one.

There is therefore a need for new molecules that can bind Gb3 and inhibit the angiogenic activity of tumor blood vessel endothelial cells, which are less toxic and have a longer duration of action.

Monoclonal antibodies may also be generated for many types of ligands, including glycosphingolipids, although some are poorly immunogenic. Monoclonal antibodies directed against Gb3 have also already been used for staining cells expressing Gb3, in particular monoclonal antibody 38.13 (Bordron et al., Chark et al.).

However, this monoclonal antibody 38.13 has been described as not binding Gb3 in the same way as the verotoxins (Chark et al.). Moreover, an anti-Gb3 monoclonal antibody 1A4 obtained from ascites has also been described as inducing apoptosis of Burkitt lymphoma cells by a different mechanism than that of verotoxin 1 (Tétaud et al.). It was therefore not very probable that an anti-Gb3 monoclonal antibody could have the same antiangiogenic effect as verotoxins.

Furthermore, WO98/51326, which describes the use of compounds binding Gb3 as antiangiogenic agents, proposes, as such agents, verotoxins and anti-Gb3 antibodies coupled to a toxic molecule, such as toxins. The authors of this document thus believed that an anti-Gb3 antibody alone would not have an antiangiogenic effect.

Moreover, Bordron et al. describe the apoptotic effect on endothelial cells of the various anti-endothelial cell antibodies (precise antigenic specificities not determined, probably mixtures of antibodies of different specificities) derived from patients with autoimmune disease, as well as several monoclonal antibodies of known specificity, including an anti-Gb3 monoclonal antibody (clone 38/13). The results presented in this document show that only 8% of endothelial cells are in a state of apoptosis after contact with anti-Gb3 monoclonal antibody 38/13, a percentage that is lower than that obtained in the presence of the medium alone, without antibodies (14%). These results also suggest that anti-Gb3 monoclonal antibodies do not have the same effect on endothelial cells as Shiga toxin or verotoxins.

However, the inventors found, surprisingly, that several anti-Gb3 monoclonal antibodies have an antiangiogenic activity on proliferating endothelial cells. These monoclonal antibodies may therefore be used in the treatment of diseases associated with angiogenesis, and particularly solid tumors, with all the advantages mentioned above that this targeting has (reduced risk of resistance, applicable to any type of tumor, targets easily reached, prevention of metastases). Moreover, they have several advantages with regard to Shiga toxin and verotoxins:
Depending on their isotypes, antibodies have a molecular mass that varies between 150 (IgG) and 1000 (IgM) kDa, and therefore a larger molecular mass than Shiga toxin and verotoxins. This should enable them to remain longer in the bloodstream and therefore have a longer duration of action than these toxins on the endothelial cells of tumor neovessels. IgG (especially IgG1) and IgM isotypes are the most advantageous. In fact, IgM antibodies have the largest molecular mass, around 80% remaining in the bloodstream, and they have an in vivo half-life of around 10 days. As for IgG, 45% remains in the bloodstream, and their half-life is around 20 days (Nikolayenko et al.). This should be compared with the fact that the administration of verotoxin 1 in rabbits shows that only 2% of verotoxin 1 is still present in the bloodstream after two hours, the rest being found in tissues expressing Gb3.
These antibodies are not toxic themselves, which should limit their side effects on other tissues expressing Gb3; the fact that they remain primarily in the bloodstream also limits these side effects.
The mouse antibodies obtained by the inventors can be humanized, so as to minimize any receiver immune reaction, which is not possible for bacterial toxins, which generate an immune response that could reduce their efficacy.

These antibodies therefore share their recognizing Gb3 and being useful in the treatment of diseases associated with angiogenesis. Moreover, two of these antibodies (3E2 and 22F6) further share their recognizing the upper band but not the lower band of the Gb3 doublet expressed by HMEC-1 cells, which upper band seems to be particularly overexpressed when endothelial cells proliferate, compared to quiescent cells. This could give them increased specificity for endothelial cells undergoing angiogenesis.

### DESCRIPTION OF THE INVENTION

Antibodies

The present invention therefore concerns an antibody directed against the membrane glycosphingolipid globotriaosylceramide (Gb3), or a functional fragment or a derivative thereof, characterized in that it has at least one complementarity determining region (CDR) with an amino acid sequence selected from SEQ ID NO: 1 to 42, or with an amino acid sequence having at least 80%, preferably at least 85%, at least 90%, at least 95%, at least 96%, at least 97%, at least 98% or at least 99% identity with one of SEQ ID NO: 1 to 42.

In one embodiment, the anti-Gb3 antibody, functional fragment or derivative according to the invention has a heavy chain comprising at least one complementarity determining region (CDR) with an amino acid sequence selected from SEQ ID NO: 1 to 3, 7 to 9, 13 to 15, 19 to 21, 25 to 27, 31 to 33, and 37 to 39, or with an amino acid sequence having at least 80%, preferably at least 85%, at least 90%, at least 95%, at least 96%, at least 97%, at least 98%, or at least 99% identity with one of SEQ ID NO: 1 to 3, 7 to 9, 13 to 15, 19 to 21, 25 to 27, 31 to 33, and 37 to 39.

In another embodiment, the anti-Gb3 antibody, functional fragment or derivative according to the invention has a light chain comprising at least on complementarity determining region (CDR) with an amino acid sequence selected from SEQ ID NO: 4 to 6, 10 to 12, 16 to 18, 22 to 24, 28 to 30, 34 to 36, and 40 to 42, or with an amino acid sequence having at least 80%, preferably at least 85%, at least 90%, at least 95%, at least 96%, at least 97%, at least 98%, or at least 99% identity with one of SEQ ID NO: 4 to 6, 10 to 12, 16 to 18, 22 to 24, 28 to 30, 34 to 36, and 40 to 42.

Advantageously, the anti-Gb3 antibody, functional fragment or derivative according to the invention has:
a heavy chain comprising at least one complementarity determining region (CDR) with at least one amino acid sequence selected from SEQ ID NO: 1 to 3, 7 to 9, 13 to 15, 19 to 21, 25 to 27, 31 to 33, and 37 to 39, or with an amino acid sequence having at least 80%, preferably at least 85%, at least 90%, at least 95%, at least 96%, at least 97%, at least 98%, or at least 99% identity with one of SEQ ID NO: 1 to 3, 7 to 9, 13 to 15, 19 to 21, 25 to 27, 31 to 33, and 37 to 39, and
a light chain comprising at least one complementarity determining region (CDR) with at least one amino acid sequence selected from SEQ ID NO: 4 to 6, to 12, 16 to 18, 22 to 24, 28 to 30, 34 to 36, and 40 to 42, or with an amino acid sequence having at least 80%, preferably at least 85%, at least 90%, at least 95%, at least 96%, at least 97%, at least 98%, or at least 99% identity with one of SEQ ID NO: 4 to 6, 10 to 12, 16 to 18, 22 to 24, 28 to 30, 34 to 36, and 40 to 42.

The anti-Gb3 antibody, functional fragment or derivative according to the invention may also advantageously have a heavy chain comprising three CDR-H (heavy chain CDR) with the following amino acid sequences, or sequences having at least 80%, at least 85%, at least 90%, at least 95%, at least 96%, at least 97%, at least 98%, or at least 99% identity with the following sequences:

a) CDR1-H-3E2: SEQ ID NO: 1, CDR2-H-3E2: SEQ ID NO: 2, CDR3-H-3E2: SEQ ID NO: 3,

b) CDR1-H-14C11: SEQ ID NO: 7, CDR2-H-14C11: SEQ ID NO: 8, CDR3-H-14C11: SEQ ID NO: 9,

c) CDR1-H-15C11: SEQ ID NO: 13, CDR2-H-15C11: SEQ ID NO: 14, CDR3-H-15C11: SEQ ID NO: 15,

d) CDR1-H-22F6: SEQ ID NO: 19, CDR2-H-22F6: SEQ ID NO: 20, CDR3-H-22F6: SEQ ID NO: 21,

e) CDR1-H-25C10: SEQ ID NO: 25, CDR2-H-25C10: SEQ ID NO: 26, CDR3-H-25C10: SEQ ID NO: 27,

f) CDR1-H-11E10: SEQ ID NO: 31, CDR2-H-11E10: SEQ ID NO: 32, CDR3-H-11E10: SEQ ID NO: 33, or

g) CDR1-H-16G8: SEQ ID NO: 37, CDR2-H-16G8: SEQ ID NO: 38, CDR3-H-16G8: SEQ ID NO: 39.

The anti-Gb3 antibody, functional fragment or derivative according to the invention may also advantageously have a light chain comprising three CDR-L (light chain CDR) with the following amino acid sequences, or sequences having at least 80%, at least 85%, at least 90%, at least 95%, at least 96%, at least 97%, at least 98%, or at least 99% identity with the following sequences:

a) CDR1-L-3E2: SEQ ID NO: 4, CDR2-L-3E2: SEQ ID NO: 5, CDR3-L-3E2: SEQ ID NO: 6,

b) CDR1-L-14C11: SEQ ID NO: 10, CDR2-L-14C11: SEQ ID NO: 11, CDR3-L-14C11: SEQ ID NO: 12,

c) CDR1-L-15C11: SEQ ID NO: 16, CDR2-L-15C11: SEQ ID NO: 17, CDR3-L-15C11: SEQ ID NO: 18,

d) CDR1-L-22F6: SEQ ID NO: 22, CDR2-L-22F6: SEQ ID NO: 23, CDR3-L-22F6: SEQ ID NO: 24,

e) CDR1-L-25C10: SEQ ID NO: 28, CDR2-L-25C10: SEQ ID NO: 29, CDR3-L-25C10: SEQ ID NO: 30,

f) CDR1-L-11E10: SEQ ID NO: 34, CDR2-L-11E10: SEQ ID NO: 35, CDR3-L-11E10: SEQ ID NO: 36, or

g) CDR1-L-16G8: SEQ ID NO: 40, CDR2-L-16G8: SEQ ID NO: 41, CDR3-L-16G8: SEQ ID NO: 42.

Still advantageously, the anti-Gb3 antibody, functional fragment or derivative according to the invention has heavy and light chains respectively comprising CDR-H and CDR-L with the following amino acid sequences, or sequences having at least 80%, at least 85%, at least 90%, at least 95%, at least 96%, at least 97%, at least 98%, or at least 99% identity with the following sequences:

a) CDR1-H-3E2: SEQ ID NO: 1, CDR2-H-3E2: SEQ ID NO: 2, CDR3-H-3E2: SEQ ID NO: 3, CDR1-L-3E2: SEQ ID NO: 4, CDR2-L-3E2: SEQ ID NO: 5, CDR3-L-3E2: SEQ ID NO: 6,

b) CDR1-H-14C11: SEQ ID NO: 7, CDR2-H-14C11: SEQ ID NO: 8, CDR3-H-14C11: SEQ ID NO: 9, CDR1-L-14C11: SEQ ID NO: 10, CDR2-L-14C11: SEQ ID NO: 11, CDR3-L-14C11: SEQ ID NO: 12,

c) CDR1-H-15C11: SEQ ID NO: 13, CDR2-H-15C11: SEQ ID NO: 14, CDR3-H-15C11: SEQ ID NO: 15, CDR1-L-15C11: SEQ ID NO: 16, CDR2-L-15C11: SEQ ID NO: 17, CDR3-L-15C11: SEQ ID NO: 18,

d) CDR1-H-22F6: SEQ ID NO: 19, CDR2-H-22F6: SEQ ID NO: 20, CDR3-H-22F6: SEQ ID NO: 21, CDR1-L-22F6: SEQ ID NO: 22, CDR2-L-22F6: SEQ ID NO: 23, CDR3-L-22F6: SEQ ID NO: 24,

e) CDR1-H-25C10: SEQ ID NO: 25, CDR2-H-25C10: SEQ ID NO: 26, CDR3-H-25C10: SEQ ID NO: 27, CDR1-L-25C10: SEQ ID NO: 28, CDR2-L-25C10: SEQ ID NO: 29, CDR3-L-25C10: SEQ ID NO: 30,

f) CDR1-H-11E10: SEQ ID NO: 31, CDR2-H-11E10: SEQ ID NO: 32, CDR3-H-11E10: SEQ ID NO: 33, CDR1-11E10L: SEQ ID NO: 34, CDR2-L-11E10: SEQ ID NO: 35, CDR3-L-11E10: SEQ ID NO: 36, or

g) CDR1-H-16G8: SEQ ID NO: 37, CDR2-H-16G8: SEQ ID NO: 38, CDR3-H-16G8: SEQ ID NO: 39, CDR1-L-16G8: SEQ ID NO: 40, CDR2-L-16G8: SEQ ID NO: 41, CDR3-L-16G8: SEQ ID NO: 42.

In one advantageous embodiment of the invention, the anti-Gb3 antibody, functional fragment or derivative according to the invention has a heavy chain comprising a variable region with a sequence selected from SEQ ID NO: 43 to 49, or with a sequence having at least 80%, preferably at least 85%, at least 90%, at least 95%, at least 96%, at least 97%, at least 98%, at least 99% identity with one of SEQ ID NO: 43 to 49.

In another advantageous embodiment of the invention, the anti-Gb3 antibody, functional fragment or derivative according to the invention has a light chain comprising a variable region having a sequence selected from SEQ ID NO: 50 to 56, or having a sequence having at least 80%, preferably at least 85%, at least 90%, at least 95%, at least 96%, at least 97%, at least 98%, at least 99% identity with one of SEQ ID NO: 50 to 56.

Advantageously, the anti-Gb3 antibody, functional fragment or derivative according to the invention has:
a heavy chain comprising a variable region with a sequence selected from SEQ ID NO: 43 to 49, or having a sequence having at least 80%, preferably at least 85%, at least 90%, at least 95%, at least 96%, at least 97%, at least 98%, at least 99% identity with one of SEQ ID NO: 43 to 49, and
a light chain comprising a variable region with a sequence selected from SEQ ID NO: 50 to 56, or having a sequence having at least 80%, preferably at least 85%, at least 90%, at least 95%, at least 96%, at least 97%, at least 98%, at least 99% identity with one of SEQ ID NO: 50 to 56.

In an advantageous embodiment, the anti-Gb3 antibody, functional fragment or derivative according to the invention may also be selected from the anti-Gb3 monoclonal antibodies generated by the inventors or variants thereof, which have heavy and light chains whose variable regions have the following amino acid sequences or sequences having at least 80%, preferably at least 85%, at least 90%, at least 95%, at least 96%, at least 97%, at least 98%, or at least 99% identity with the following sequences:

a) Antibody 3E2: heavy chain: SEQ ID NO: 43, light chain: SEQ ID NO: 50,

b) Antibody 14C11: heavy chain: SEQ ID NO: 44, light chain: SEQ ID NO: 51,

c) Antibody 15C11: heavy chain: SEQ ID NO: 45, light chain: SEQ ID NO: 52,

d) Antibody 22F6: heavy chain: SEQ ID NO: 46, light chain: SEQ ID NO: 53,

e) Antibody 25C10: heavy chain: SEQ ID NO: 47, light chain: SEQ ID NO: 54,

f) Antibody 11E10: heavy chain: SEQ ID NO: 48, light chain: SEQ ID NO: 55, and

g) Antibody 16G8: heavy chain: SEQ ID NO: 49, light chain: SEQ ID NO: 56.

“Antibody” or “immunoglobulin” means a glycoprotein composed of two types of glycopeptide chains called “heavy chain” and “light chain”, an antibody being made up of two heavy chains and two light chains, bound by disulfide bridges. Each chain is made up of a variable region and a constant region. The constant region of a particular isotype of heavy or light chain is normally identical from one antibody to another of the same isotype, excluding somatic mutations. In return, the variable region varies from one antibody to another. Indeed, genes coding for antibody heavy chains and light chains are generated by recombination of, respectively, three and two segments of distinct genes called VH, DH and JH-CH for the heavy chain and VL and JL-CL for the light chain. The CH and CL segments do not participate in recombination and form the constant regions of the heavy and light chains respectively. Recombinations of the VH-DH-JH and VL-JL segments form the variable regions of heavy and light chains, respectively. The VH and VL regions have three hypervariable zones or complementarity determining regions (CDR) called CDR1, CDR2 and CDR3, the CDR3 region being the most variable, since it is located at the recombination zone. These three CDR regions, and particularly the CDR3 region, are found in the part of the antibody that will be in contact with the antigen and are therefore very important for antigen recognition. Thus, antibodies maintaining the three CDR regions and each of the heavy and light chains of an antibody mostly keep the antigenic specificity of the original antibody. In a certain number of cases, an antibody only maintaining one of the CDRs, and particularly CDR3, also keeps the specificity of the original antibody. The CDR1, CDR2 and CDR3 regions are each preceded by FR1, FR2 and FR3 regions, respectively, corresponding to framework regions (FR) which vary from one VH or VL segment to another. The CDR3 region is also followed by a framework region FR4.

The CDRs of an antibody are defined from the amino acid sequence of its heavy and light chains compared to criteria known to the skilled person. Various methods for determining CDRs have been proposed, and the portion of the amino acid sequence from a heavy or light chain variable region of an antibody defined as a CDR varies depending on the method chosen. The first determination method is the one proposed by Kabat et al. (Kabat et al. Sequences of proteins of immunological interest, 5^{th} Ed., U.S. Department of Health and Human Services, NIH, 1991, and later editions). In this method, CDRs are defined by looking for the amino acids responsible for binding the antigen to the antibody. A second method was proposed by the IMGT, based on determining hypervariable regions. In this method, a unique numbering has been defined to compare variable regions regardless of the antigen receptor, the chain type or the species (Lefranc et al. 2003). This numbering provides a standardized definition of framework regions ((FR1-IMGT: positions 1 to 26, FR2-IMGT: 39 to 55, FR3-IMGT: 66 to 104 and FR4-IMGT: 118 to 128) and complementarity determining regions (CDR1-IMGT: positions 27 to 38, CDR2-IMGT: positions 56 to 65 and CDR3-IMGT: positions 105 to 117). Finally, there is also a numbering called “common” in which the sequence of a particular CDR corresponds to the common sequence between the Kabat numbering and the IMGT numbering. Throughout the present description, the CDR sequences are indicated by the IMGT numbering. In particular, CDRs have been determined by using the IMGT/V-QUEST program available at http://imgt.cines.fr and described in Brochet et al. (2008).

Table 2 below summarizes the amino acid sequences of the CDRs and variable regions of the heavy and light chains of anti-Gb3 antibodies generated by the inventors:

Antibody 3E2
Heavy chain

| | |
|---|---|
| CDR1 | SEQ ID NO: 1 |
| CDR2 | SEQ ID NO: 2 |
| CDR3 | SEQ ID NO: 3 |
| Variable region | SEQ ID NO: 43 |

Light chain

| | |
|---|---|
| CDR1 | SEQ ID NO: 4 |
| CDR2 | SEQ ID NO: 5 |
| CDR3 | SEQ ID NO: 6 |
| Variable region | SEQ ID NO: 50 |

Antibody 14C11
Heavy chain

| | |
|---|---|
| CDR1 | SEQ ID NO: 7 |
| CDR2 | SEQ ID NO: 8 |
| CDR3 | SEQ ID NO: 9 |
| Variable region | SEQ ID NO: 44 |

Light chain

| | |
|---|---|
| CDR1 | SEQ ID NO: 10 |
| CDR2 | SEQ ID NO: 11 |
| CDR3 | SEQ ID NO: 12 |
| Variable region | SEQ ID NO: 51 |

Antibody 15C11
Heavy chain

| | |
|---|---|
| CDR1 | SEQ ID NO: 13 |
| CDR2 | SEQ ID NO: 14 |
| CDR3 | SEQ ID NO: 15 |
| Variable region | SEQ ID NO: 45 |

Light chain

| | |
|---|---|
| CDR1 | SEQ ID NO: 16 |
| CDR2 | SEQ ID NO: 17 |
| CDR3 | SEQ ID NO: 18 |
| Variable region | SEQ ID NO: 52 |

Antibody 22F6
Heavy chain

| | |
|---|---|
| CDR1 | SEQ ID NO: 19 |
| CDR2 | SEQ ID NO: 20 |
| CDR3 | SEQ ID NO: 21 |
| Variable region | SEQ ID NO: 46 |

Light chain

| | |
|---|---|
| CDR1 | SEQ ID NO: 22 |
| CDR2 | SEQ ID NO: 23 |
| CDR3 | SEQ ID NO: 24 |
| Variable region | SEQ ID NO: 53 |

Antibody 25C10
Heavy chain

| | |
|---|---|
| CDR1 | SEQ ID NO: 25 |
| CDR2 | SEQ ID NO: 26 |
| CDR3 | SEQ ID NO: 27 |
| Variable region | SEQ ID NO: 47 |

Light chain

| | |
|---|---|
| CDR1 | SEQ ID NO: 28 |
| CDR2 | SEQ ID NO: 29 |
| CDR3 | SEQ ID NO: 30 |
| Variable region | SEQ ID NO: 54 |

Antibody 11E10
Heavy chain

| | |
|---|---|
| CDR1 | SEQ ID NO: 31 |
| CDR2 | SEQ ID NO: 32 |
| CDR3 | SEQ ID NO: 33 |
| Variable region | SEQ ID NO: 48 |

Light chain

| | |
|---|---|
| CDR1 | SEQ ID NO: 34 |
| CDR2 | SEQ ID NO: 35 |
| CDR3 | SEQ ID NO: 36 |
| Variable region | SEQ ID NO: 55 |

Antibody 16G8
Heavy chain

| | |
|---|---|
| CDR1 | SEQ ID NO: 37 |
| CDR2 | SEQ ID NO: 38 |
| CDR3 | SEQ ID NO: 39 |
| Variable region | SEQ ID NO: 49 |

Light chain

| | |
|---|---|
| CDR1 | SEQ ID NO: 40 |
| CDR2 | SEQ ID NO: 41 |
| CDR3 | SEQ ID NO: 42 |
| Variable region | SEQ ID NO: 56 |

“Functional fragment” means an antibody conserving the antigen binding domain and therefore having the same antigenic specificity as the original antibody, such as fragments Fv, ScFv, Fab, F(ab′)2, Fab′, scFv-Fc or diabodies).

A “derivative” of an antibody means a binding protein formed of a support peptide and at least one CDR of the original antibody preserving its ability to recognize Gb3.

The antibodies, functional fragments or derivatives according to the invention can be obtained by genetic recombination or chemical synthesis, according to technologies well known to the skilled person.

According to a preferred embodiment, the antibody according to the invention is a monoclonal antibody. “Monoclonal” means an antibody obtained from a substantially homogenous population of antibodies, that is, the antibodies forming this population are essentially identical with the possible exception of natural mutations that may be present in small quantities. These antibodies are directed against a single epitope and are consequently very specific. “Epitope” means the site on the antigen where the antibody binds. Regardless of the antigen, an epitope is made up of a three-dimensional region formed by parts of the antigen which may or may not be adjacent in the linear, non-three-dimensional structure of the antigen.

The antibodies, functional fragments or derivatives are, of course, in an isolated rather than natural form, obtained by purification from a natural source, by genetic recombination or even by chemical synthesis.

In the sense of the present description, the “percentage of identity” between two nucleic acid or amino acid sequences means the percentage of nucleotides or amino acids identical between the two compared sequences, obtained after optimal alignment of both sequences. This percentage is purely statistical and the differences between the two sequences are randomly distributed over their length. The two nucleic acid sequences or amino acid sequences are generally compared after they are optimally aligned; the comparison can be done by using an “alignment window”. The sequences can be optimally aligned by using various software well known to the skilled person, including BLAST NR (nucleic acids) or BLAST P (proteins).

The percentage of identity between two nucleic acid or amino acid sequences is determined by comparing the two optimally-aligned sequences, in which the nucleic acid sequences or amino acid sequences compared may have deletions or insertions compared to the reference sequence. The percentage of identity is calculated by determining the number of positions at which the nucleotide or amino acid is identical between two sequences, preferably between two complete sequences, and dividing it by the total number of positions in the alignment window (preferably complete sequences) and multiplying the result by 100. This percentage of identity may be easily calculated by using BLAST software, for example, with the default parameters.

When the CDR or variable region of an antibody according to the invention has an amino acid sequence that is not 100% identical to one of those described above and in the sequence listing (reference sequences) but which has at least 80%, preferably at least 85%, at least 90%, at least 95%, at least 96%, at least 97%, at least 98%, at least 99% identity with one of the reference sequences, it may have insertions, deletions or substitutions with regard to the reference sequence. When it is a matter of substitutions, the substitution is preferably done with an “equivalent” amino acid, that is to say any amino acid whose structure is similar to the original amino acid and therefore unlikely to change the biological activity of the antibody. Examples of such substitutions are presented in Table 3 below:

TABLE 3
Substitutions with equivalent amino acids

| Original amino acid | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

All antibodies mentioned above recognize Gb3. In the human HMEC-1 cells used by the inventors, Gb3 is represented in immunostaining by a doublet corresponding to the presence of two different fatty acid chains at the ceramide. Pig cells also express two forms of Gb3 represented by a doublet. Antibody 3E2 recognizes both forms of Gb3 of the pig doublet, as well as the upper band, but not the lower band, of the doublet expressed by HMEC-1 cells. Preliminary results suggest that the upper band of the Gb3 doublet expressed by HMEC-1 cells is over-expressed in HMEC-1 cells during angiogenesis compared to quiescent HMEC-1 cells (see FIG. 14, where the percentage of cells recognized by antibody 3E2 specific for this upper band increases when the cells are in complete medium, while the percentage of cells recognized by antibody 1A4, which indifferently recognizes both bands, does not vary). Consequently, the specificity of antibody 3E2, and any antibody with the same CDR 1, CDR2 and CDR3 or the same variable regions for this upper band likely further increases its specificity for proliferating endothelial cells, compared to quiescent endothelial cells, which is likely to further decrease its side effects. Antibody 22F6 also recognizes this upper band of the Gb3 doublet expressed by HMEC-1 cells and should therefore be especially specific for proliferating endothelial cells, as well as any antibody having the same CDR 1, CDR2 and CDR 3 or the same variable regions. This is not the case for antibodies 38.3 and 1A4 of the prior art, which recognize both bands of the doublet (see FIGS. 7 and 8), antibody 38.3 even seeming to mainly recognize the lower band of the doublet (see FIG. 7-I). Thus, in an advantageous embodiment, the antibody, functional fragment or derivative according to the invention is antibody 3E2 or 22F6 such as defined above by the sequences of the variable regions of the heavy and light chains, advantageously antibody 3E2, or an antibody of the same antigenic specificity. Notably, the antibody, functional fragment or derivative according to the invention may have at least one complementarity determining region (CDR) with an amino acid sequence selected from SEQ ID NO: 1 to 6 and 19 to 24, or with an amino acid sequence having at least 80%, preferably at least 85%, at least 90%, at least 95%, at least 96%, at least 97%, at least 98%, at least 99% identity with one of SEQ ID NO: 1 to 6 and 19 to 24. More precisely, the antibody, functional fragment or derivative according to the invention may have at least one complementarity determining region (CDR) with an amino acid sequence selected from SEQ ID NO: 1 to 3 and 19 to 21, or with an amino acid sequence having at least 80%, preferably at least 85%, at least 90%, at least 95%, at least 96%, at least 97%, at least 98%, at least 99% identity with one of SEQ ID NO: 1 to 3, and 19 to 21 and/or a light chain comprising at least one complementarity determining region (CDR) with at least one amino acid sequence selected from SEQ ID NO: 4 to 6 and 22 to 24, or with an amino acid sequence having at least 80%, preferably at least 85%, at least 90%, at least 95%, at least 96%, at least 97%, at least 98%, at least 99% identity with one of SEQ ID NO: 4 to 6 and 22 to 24. It may also advantageously have a heavy chain comprising three CDR-H (heavy chain CDR) having the following amino acid sequences, or sequences having at least 80%, preferably at least 85%, at least 90%, at least 95%, at least 96%, at least 97%, at least 98%, or at least 99% identity with the following sequences:

a) CDR1-H-3E2: SEQ ID NO: 1, CDR2-H-3E2: SEQ ID NO: 2,

b) CDR1-H-22F6: SEQ ID NO: 19, CDR2-H-22F6: SEQ ID NO: 20, CDR3-H-22F6: SEQ ID NO: 21,

and/or have a light chain comprising three CDR-L (light chain CDR) having the following amino acid sequences, or sequences having at least 80%, preferably at least 85%, at least 90%, at least 95%, at least 96%, at least 97%, at least 98%, or at least 99% identity with the following sequences:

a) CDR1-L-3E2: SEQ ID NO: 4, CDR2-L-3E2: SEQ ID NO: 5, CDR3-L-3E2: SEQ ID NO: 6,

b) CDR1-L-22F6: SEQ ID NO: 22, CDR2-L-22F6: SEQ ID NO: 23, CDR3-L-22F6: SEQ ID NO: 24.

Advantageously, the anti-Gb3 antibody, functional fragment or derivative according to the invention can have:
a heavy chain comprising a variable region with a sequence selected from SEQ ID NO: 43 and 46, or having a sequence having at least 80%, preferably at least 85%, at least 90%, at least 95%, at least 96%, at least 97%, at least 98%, at least 99% identity with one of SEQ ID NO: 43 and 46, and/or
a light chain comprising a variable region with a sequence selected from SEQ ID NO: 50 and 53 or a sequence having at least 80%, preferably at least 85%, at least 90%, at least 95%, at least 96%, at least 97%, at least 98%, at least 99% identity with one of SEQ ID NO: 50 and 53.

In one particularly advantageous embodiment, the anti-Gb3 antibody, functional fragment or derivative according to the invention may particularly be selected from the anti-Gb3 monoclonal antibodies generated by the inventors or variants thereof, which have heavy and light chains whose variable regions have the following amino acid sequences or sequences having at least 80%, preferably at least 85%, at least 90%, at least 95%, at least 96%, at least 97%, at least 98%, or at least 99% identity with the following sequences:

a) Antibody 3E2: heavy chain: SEQ ID NO: 43, light chain: SEQ ID NO: 50, and

b) Antibody 22F6: heavy chain: SEQ ID NO: 46, light chain: SEQ ID NO: 53.

As indicated in the introduction, the most advantageous isotypes are the IgG isotypes, especially IgG1, and IgM isotypes, more particularly the IgM isotype, which has the highest molecular weight. Consequently, any antibody according to the invention is advantageously of isotype IgG or IgM, preferably IgM.

Moreover, any antibody, functional fragment or derivative according to the invention can advantageously be chimeric or humanized. This prevents patient immune reactions against the antibody administered. In particular, the antibody according to the invention can advantageously be any one of the chimeric or humanized versions of antibodies 3E2, 14C11, 15C11, 22F6, 25C10, 11E10, and 16G8 described above, advantageously antibodies 3E2 and 22F6, and especially antibody 3E2.

“Chimeric” antibody means an antibody that contains a natural variable region (light chain and heavy chain) derived from an antibody from a given species in combination with the constant regions of the light chain and heavy chain of an antibody of a species heterologous to said given species. Chimeric antibodies according to the invention can be prepared by using genetic recombinant techniques. For example, chimeric antibodies can be made by cloning recombinant DNA bearing a promoter and a sequence coding for the variable region of a non-human monoclonal antibody, particularly murine, according to the invention and a sequence coding for the constant region of a human antibody. A chimeric antibody of the invention encoded by such a recombinant gene will be, for example, a mouse-human chimera, the specificity of this antibody being determined by the variable region derived from mouse DNA and its isotype determined by the constant region derived from human DNA. This will notably be the case of chimeric antibodies obtained from murine monoclonal antibodies 3E2, 14C11, 15C11, 22F6, 25C10, 11E10, and 16G8 described in the present description. For chimeric antibody preparation methods, refer, for example, to the document Verhoeyn et al. (BioEssays, 8: 74, 1988).

“Humanized” antibody means an antibody that contains CDRs derived from an antibody of non-human origin, the other parts of the antibody molecule being derived from one (or more) human antibodies. Moreover, some skeleton segment residues (called FR) can be modified to conserve binding affinity (Jones et al. Nature, 321: 522-525, 1986; Verhoeyen et al. Science, 239: 1534-1536, 1988; Riechmann et al. Nature, 332: 323-327, 1988). Humanized antibodies according to the invention may be prepared from techniques known to the skilled person such as the technologies of CDR grafting, resurfacing, superhumanization, human string content, FR libraries, Guided selection, FR shuffling and humaneering, as summarized in the review by Almagro et al.

The anti-Gb3 antibody, or functional fragment or derivative according to the invention can also be optimized for certain effector functions. Thus, it can particularly comprise mutations increasing its affinity for the Fc receptor to which its isotype binds. It can also be produced under particular conditions (cells, media, etc.) to obtain a specific glycosylation of the antibody. Notably, for IgG, some substitutions of the Fcγ portion as well as a weak or null fucosylation increase the affinity for the FcγRIII receptor (see, in particular, Shields et al. Journal of Biological Chemistry. Vol. 276, No. 9, Issue of March 2, pp. 6591-6604, 2001, EP1176195A1, EP1331266A1, WO 01/77181). Alternatively or additionally, the antibody can also, in particular when it is an IgG isotype and especially an IgG1 isotype, have been modified to limit its ability to bind complement and therefore its complement-dependent cytotoxicity (CDC) while preserving its antibody dependent cellular cytotoxicity (ADCC). In the case of an anti-GD2 antibody, it has been shown that a point mutation of the constant part of the human IgG1 region (replacing the lysine in position 322 of the human constant part Fc of the κ light chain by an alanine) reduces its ability to bind complement while maintaining its ADCC properties, and decreases some of the side effects associated with the administration of this antibody (U.S. Pat. No. 7,432,357B2). Thus, in one advantageous embodiment, the anti-Gb3 antibody according the invention is an antibody with low CDC activity, i.e., in an in-vitro CDC activity test, the CDC activity of the antibody at a concentration of 0.1 μg/ml is not significantly different from that of the control without antibody. In particular, one advantageous embodiment concerns a humanized isotype IgG1 anti-Gb3 antibody, comprising a κ isotype light chain, in which the lysine in position 322 of the human constant part Fc of the κ light chain is replaced by an alanine or an equivalent amino acid, preferably by an alanine.

The invention also concerns a nucleic acid (also called nucleic or nucleotide sequence) encoding any of the anti-Gb3 antibodies, or functional fragments or derivatives according to the invention as described above. All the different nucleic sequences, due to the degeneracy of genetic code, encoding a particular amino acid sequence are within the scope of the invention. Such nucleic sequences may have been optimized to promote expression in a host cell of interest. Sequences of particular interest are those of the variable regions of the heavy and light chains of the antibodies generated by the inventors, represented as follows:
Antibody 3E2: heavy chain: SEQ ID NO: 57, light chain: SEQ ID NO: 64,
Antibody 14C11: heavy chain: SEQ ID NO: 58, light chain: SEQ ID NO: X65
Antibody 15C11: heavy chain: SEQ ID NO: 59, light chain: SEQ ID NO: 66,
Antibody 22F6: heavy chain: SEQ ID NO: 60, light chain: SEQ ID NO: 67
Antibody 25C10: heavy chain: SEQ ID NO: 61, light chain: SEQ ID NO: 68,
Antibody 11E10: heavy chain: SEQ ID NO: 62, light chain: SEQ ID NO: 69,
Antibody 16G8: heavy chain: SEQ ID NO: X63, light chain: SEQ ID NO: 70.

The invention also concerns a vector comprising at least one of the nucleic acid sequences described above. Such a vector includes the elements necessary to the expression of said nucleic sequence, and notably a promoter, a transcription initiation codon, termination sequences and appropriate transcription regulation sequences. These elements vary depending on the host used for expression and are easily selected by the skilled person in view of his general knowledge. The vector can notably be a plasmid or virus. It is used to clone or express the nucleic sequences according to the invention.

The invention also concerns a host cell comprising one or more nucleic sequences or one or more vectors according to the invention. The host cell can be of prokaryotic or eukaryotic origin, and may notably be selected from bacterial cells, insect, plant, yeast or mammals cells. The antibody, functional fragment or derivative according to the invention may then be produced by culturing the host cell under appropriate conditions. It may also be obtained by chemical synthesis, notably in the solid or semisolid phase.

The antibodies, functional fragments or derivatives according to the invention have antiangiogenic properties as such. Notably, they have a cytostatic and cytotoxic effect on proliferating endothelial cells. They may therefore be used alone in the treatment of diseases associated with angiogenesis, such as defined below. Thus, in one embodiment, the antibodies according to the invention described above are not coupled to a therapeutic molecule, especially a cytotoxic, cytostatic or antiangiogenic molecule such as a toxin, in particular a toxin inhibiting angiogenesis. In the sense of the present invention, an antibody is “coupled” to a molecule if there is a covalent bond between the antibody and the molecule. Thus, the fact that the antibody according to the invention is not coupled to a therapeutic molecule means that it is not bound by a covalent bond to such a molecule. However, this does not prevent the antibody, not coupled to a therapeutic molecule, from being administered in combination (simultaneously or sequentially) with a therapeutic molecule including a cytotoxic, cytostatic or antiangiogenic molecule in the case of solid tumors, the two molecules being able to act independently of one another.

In another embodiment, the anti-Gb3 antibodies or functional fragments or derivatives thereof according to the invention such as described above may nevertheless be coupled to another therapeutic molecule.

Treatment of Diseases Associated with Angiogenesis

The present invention also concerns an antibody directed against the membrane glycosphingolipid Gb3 and not coupled to a therapeutic molecule, for its use as a drug in the treatment of diseases associated with angiogenesis. It also concerns the use of an antibody directed against the membrane glycosphingolipid Gb3 and not coupled to a therapeutic molecule, for preparation of a drug intended to treat diseases associated with angiogenesis. It also concerns a method of treatment for diseases associated with angiogenesis in a subject in need thereof, comprising the administration of an effective quantity of an antibody directed against the membrane glycosphingolipid Gb3 and not coupled to a therapeutic molecule. It also concerns a method to inhibit angiogenesis in a subject in need thereof, comprising the administration of an effective quantity of an antibody directed against the membrane glycosphingolipid Gb3 and not coupled to a therapeutic molecule.

“Diseases associated with angiogenesis” means any disease that requires angiogenesis to progress, i.e., the formation of new vessels from a pre-existing vascular network. These diseases associated with angiogenesis can include solid tumors; psoriasis; angiomas; proliferative eye diseases, including age-related macular degeneration (AMD), diabetic retinopathy or neovascular glaucoma; autoimmune diseases such as lupus or rheumatoid arthritis and diseases related to atherosclerosis, obesity or Alzheimer's disease. Indeed, the development of all these diseases involves angiogenesis. Of course, blood cell diseases, such as leukemia or lymphoma, are liquid tumors, which do not require angiogenesis, and are therefore not considered diseases associated with angiogenesis in the sense of the present invention. In an advantageous embodiment, anti-Gb3 antibody, not bound to a cytotoxic, cytostatic or antiangiogenic molecule, is used in the treatment of solid tumors.

Solid tumors for which anti-Gb3 treatment is useful include adenomas, sarcomas and carcinomas; and especially adenocarcinomas, ovarian, breast, pancreatic, skin, lung, brain, kidney, liver, nasopharyngeal cavity, thyroid, central nervous system (neuroblastoma, for example), prostate, colon, rectum, cervical, testicular or bladder cancer.

In an advantageous embodiment of the application to the treatment of diseases associated with angiogenesis, especially for the treatment of solid tumors, the anti-Gb3 antibody used is one of those described above in the part concerning antibodies as such.

In another advantageous embodiment, the anti-Gb3 antibody recognizes the upper band but not the lower band of the Gb3 doublet expressed by HMEC-1 cells. This may particularly be antibodies 22F6 and 3E2, or an antibody having at least one CDR, or all the CDRs, or even one and/or the other variable region of these antibodies.

As part of the treatment of solid cancers, anti-Gb3 antibodies may be administered in combination with another treatment, notably surgical resection of the tumor, treatment with radiation therapy or chemotherapy with another therapeutic molecule, as long as the other therapeutic molecule is not coupled to the antibody. The two treatments (antibody and surgery/radiation therapy/other therapeutic molecule) may then be administered simultaneously (i.e., at the same time, in a same composition or in two separate compositions) or sequentially (one then the other, possibly alternating). They can also be administered first simultaneously and then sequentially, or vice versa. The combination may also be administered first (simultaneously or sequentially) then treatment can be continued with only one of the two therapies. Provided a therapeutic molecule is not coupled to the antibody, it can be selected from all other anticancer molecules, whether they also target angiogenesis or the tumor itself.

### DESCRIPTION OF THE FIGURES

FIG. 1. (A) HPTLC profiles of HMEC-1 cells in the growth (lane 1) or confluent phase (lane 2), rat brain ganglioside markers (lane 3) and standard neutral glycolipids (lane 4). (B) HPTLC profiles of HMEC-1 cells incubated in depleted culture medium (lane 1) or complete culture medium (lane 2), rat brain ganglioside markers (lane 3) and mixtures of standard neutral glycosphingolipids (lane 4). (C) HPTLC profiles of HMEC-1 cells treated by PET (lane 1) or S1P (lane 2), rat brain ganglioside markers (lane 3) and standard neutral glycolipids (lane 4). The migration solvent used is 0.2% C/M/H₂O, CaCl₂, 55:45:10 v/v/v. All the bands are revealed by orcinol.

FIG. 2. Protocol for obtaining antibodies directed against HMVEC-L animal cells cultured in the presence of T84 human adenocarcinoma cells.

FIG. 3. Principle of cloning by limiting dilution in 96-well microplates. (Panel A). 1000 hybridoma cells are seeded in position A1, which are then two-fold serially diluted from top to bottom, wells A to H, then left to right, wells 1 to 12. Panel B shows the theoretical number of cells remaining after dilution.

FIG. 4. Electrophoretic analysis of IgM mAb 3E2 on a 6% polyacrylamide gel under non-reducing conditions (A) and on a 12% polyacrylamide gel under reducing conditions (B).

FIG. 5. Analysis of staining by flow cytometry of HMEC-1, Raji and NXS2 cells with purified monoclonal anti-Gb3 antibodies 3E2.c (A), 25C10.a (B), 16G8.h (C), 14C11.1d (D), 15C7.2e (E). All the cells were stained with 10 μg/ml of antibody. The percentages of positive cells are noted in the figures for panel A, B, C, D, E and F.

FIG. 6. ELISA study of the specificity of antibody 3E2 (Panel A). Binding of antibody 3E2 measured by ELISA on Raji cells, HMEC-1 cells and IMR32 cells. (Panel B) Binding of antibody 3E2 and rat anti-Gb3 monoclonal antibody 38.13 on HMEC-1 cells measured by ELISA. The initial antibody concentration is equal to 10 μg/ml (3 independent analyses).

FIG. 7. Immunostaining on HPTLC of HMEC-1 cells by mAb 3E2, mAb 38.13 and the supernatant of the uncloned 3E2 hybridoma. Rat brain ganglioside markers (lane 1) and glycolipid extracts of HMEC-1 cells (lanes 2, 3, 4 and 5). (A) The bands are chemically revealed by orcinol. (B) Immunostaining is done with 5 μg/ml of rat anti-Gb3 mAb 38-13 (lane 3), 5 μg/ml mAb 3E2 (lane 4) and the supernatant of hybridoma 3E2 before cloning (lane 5).

FIG. 8. Immunostaining on HPTLC of HMEC-1 cells by mouse mAb 3E2 and 1A4. Rat brain ganglioside markers (lane M), mixture of neutral glycolipids (lane 1), purified pig Gb3 (lane 2) and HMEC-1 cell glycolipid extracts (lane 3). (Panel A). The lines are chemically revealed by orcinol. (Panel B) Immunostaining is done with 5 μg/ml mAb 3E2 or 1A4.

FIG. 9. Analysis of the specificity of antibody 3E2 by flow cytometry on HMEC-1, Raji and NXS2 cells. Expression of Gb3 on HMEC-1, Raji and NXS2 cells by flow cytometry with 10 μg/ml of mAb 3E2 (A), 1A4 (B) and isotype control IgM, (C). The percentages of positive cells are noted in Table 10.

FIG. 10. Glycolipid profile by HPTLC revealed with orcinol of HMEC-1 (4), Raji (5), NXS2 (6) and T84 (7) cells. Rat brain ganglioside markers (lane 1), mixture of standard neutral glycolipids (lane 2), and purified pig Gb3 (lane 3).

FIG. 11. Expression of Gb3 in HMEC-1, HMVEC-L and HUVEC cells by flow cytometry with 10 μg/ml of mAb 3E2 (A), 1A4 (B) and isotype control IgM, (C). The percentages of positive cells are noted in FIG. 11 and the mean fluorescence values are listed in Table 11.

FIG. 12. Glycolipid profile by HPTLC revealed with orcinol of HMEC-1 (4), HMVEC-L (5) and HUVEC (6) cells. Rat brain ganglioside markers (lane 1), mixture of standard neutral glycolipids (lane 2), and purified pig Gb3 (lane 3).

FIG. 13. Nucleotide sequences of the variable regions of the V_{H} (panel A) and V_{L} (panel B) gene segments of antibody 3E2.

FIG. 14. Flow cytometry analysis of the expression of Gb3 in HMEC-1 cells incubated for 24 h in depleted medium (IM) and complete medium (CM). Binding was measured by means of mAb 3E2 (Panel A) and mAb 1A4 (Panel B). The percentage of positive cells and their mean fluorescence intensity values are listed in Table 14 (three independent experiments).

FIG. 15. Flow cytometry analysis of the expression of Gb3 in HMEC-1 cells incubated in depleted medium supplemented with 1 μM SP or PET. Binding was measured by means of mAb 3E2 at 24 hours (Panel A) and at 72 hours (Panel B). The percentage of positive cells and their mean fluorescence intensity values are listed in Table 15 (three independent experiments).

FIG. 16. Cellular viability of HMEC-1, Raji and NXS2 cells measured by MTT after 24 h of incubation of mAb 3E2 and 1A4. Cellular viability of HMEC-1 cells in the presence of mAb 3E2 and 1A4 (Panel A) and Raji and NXS2 cells in the presence of mAb 3E2 (panel B), 3 independent experiments.

FIG. 17. Cellular viability kinetics of HMEC-1 cells measured by MTT after 24 h, 48 h and 72 h of incubation of mAb 3E2 at 20 μg/ml (3 independent experiments).

FIG. 18. Representation of the number of HMEC-1 cells as a function of the incubation time of mAb 3E2 at 20 μg/ml.

FIG. 19. Percentage of dividing HMEC-1 cells and estimate of their cell division time in the presence of antibody 3E2. (Panel A) Proportion of cells that enter into cell division compared to the initial number of cells, as a function of incubation time (n=3). (Panel B) Cell division time: proportion of cells entered into division as a function of their cell division time, compared to the total number of cell divisions over 24 h of incubation (n=3).

FIG. 20. Aortic ring test done with mAb 3E2 and 1A4 (n=3). (Panel A) Developing microvessels from cross sections of mouse aortic rings. Photo 1, growth of microvessels at implantation of aortic sections (D0). Photo 2, growth of microvessels at the end of five days after incubation in complete culture medium. Photos 3 and 4, aortic rings treated with, respectively, 20 μg/ml and 40 μg/ml mAb 3E2. (Panel B) Scores assigned to the length, density and number of vascular buds from analysis of aortic ring images (scale from 0 to 5, n=5).

FIG. 21. Evaluation of the CDC activity of mAb 3E2 and 1A4. Specific cell lysis was determined by flow cytometry by measuring the percentage of cells that incorporated propidium iodide. The CDC activity is measured in the presence of decomplemented (dc′) or non decomplemented (c′) human serum. (Panel A) CDC activity on HMEC-1 cells, (Panel B) Raji cells and (Panel C) NXS2 cells.

FIG. 22. Nucleotide sequences of the variable regions of the heavy chain of 7 anti-Gb3 antibodies.

FIG. 23. Nucleotide sequences of the variable regions of the light chain of 7 anti-Gb3 antibodies.

FIG. 24. Reduction in the growth of a subcutaneous tumor by antibody 3E2. Female AJ mice were inoculated subcutaneously in the right flank with 10⁶ living NXS2 cells (neuroblastoma line) (viability >95%) resuspended in 150 μL of PBS. When the tumors reached a volume V of 100-150 mm³, the mice received a single injection of antibody 3E2 directed against the upper band of the Gb3 doublet expressed by HMEC-1 cells, anti-GD2 antibody 14G2a, an isotype IgM control antibody or PBS alone. The graph represents the tumor volume, expressed in percentage of the tumor volume during the treatment injection, as a function of the duration after treatment.

FIG. 25. Antibody 3E2 inhibits in vivo growth of metastases. (A) Photographs representative of mice livers at 28 days after intravenous injection of NXS2 cells. The bars of the scale represent 1 cm. (B) Number of metastases evaluated for each animal liver (PBS n=4, 3E2 n=6; 14G2a n=4; control IgM n=13; mean±SEM; * p<0.05).

### EXAMPLES

### Example 1

### Materials and Methods for Examples 2 to 6

1.1 Cell Culture

Endothelial Cells: HMVEC-L, HMEC-1, HUVEC.

HMVEC-L cells are primary human microvascular endothelial cells from lungs (Cambrex, Clonetics®, USA). Each ampule corresponds to a single Caucasian donor. They are seeded at a density of 5 10⁴ cells/cm² on 6-well plates, then cultured in a humid atmosphere enriched in CO₂ at 37° C. in the specific culture medium EGM®-2 MV (Cambrex, USA) containing 25 ml of FCS (fetal calf serum), 0.2 ml of hydrocortisone, 2 ml of hFGF-B, 0.5 ml of VEGF, 0.5 ml of R3-IGF-1, 0.5 ml of ascorbic acid, 0.5 ml of hEGF and 0.5 ml of GA-1000, per 500 ml of medium. They are supplied at the third passage and may be cultured until the seventh passage. The HMEC-1 (human microvascular endothelial cell) line was obtained by the team of Prof. F. J. Candal (Center for Disease Control, Atlanta; Ades et al. 1992) after immortalization of microvascular endothelial cells from human skin by transfection with a plasmid containing the region coding for the T antigen of SV40 virus. These cells conserve the main characteristics of microvascular endothelial cells (Ades et al. 1992) and are cultured between passages 13 and 20 in order to limit the phenotype drift associated with cell culture. They are seeded at a cellular density of 2 10⁴ cells/cm² and cultured for 5 days until confluence is reached in a humid atmosphere enriched with CO₂ at 37° C. in MCDB 131 culture medium (Invitrogen, Cergy-Pontoise, France) comprising 15% inactivated FCS at 56° C. for 45 min, 10 ng/ml EGF (Becton-Dickinson, BD Biosciences, Le Pont de Claix, France), 2 μg/ml of hydrocortisone (Sigma-Aldrich, Saint Quentin-Favier, France), 2 mM of L-glutamine (Gibco-BRL, Cergy Pontoise, France), 100 IU/ml of penicillin and 100 μg/ml of streptomycin (Gibco-BRL). HUVEC cells are primary human macrovascular cells extracted from umbilical cord veins (Cambrex). Each ampule corresponds to several donors. They are seeded at a density of 3.5 10⁴ cells/cm² in 6-well plates, then cultured in a humid atmosphere enriched with CO₂ at 37° C. in the following specific culture medium EGM™-2 (Cambrex, USA): 10 ml of FCS, 0.2 ml of hydrocortisone, 2 ml of hFGF-B, 0.5 ml of VEGF, 0.5 ml of R3-IGF-1, 0.5 ml of ascorbic acid, 0.5 ml of hEGF, 0.5 ml of GA-1000 and 0.5 ml heparin, per 500 ml of medium. They may be cultured from the first to the sixth passage.

Tumor Lines: Raji, NXS2, IMR32 and T84.

The human Raji cells and the human neuroblastoma cell line IMR32 come from ATCC (Rockville, USA). The mouse neuroblastoma NXS2 cells were provided to us by Prof. H. Lode (Charité Children's Hospital, Berlin, Germany). Raji cells are seeded in an amount of 0.1 10⁶ cells/ml in RPMI 1640 medium (Invitrogen) supplemented with 10% inactivated FCS, 2 mM of L-glutamine, 100 IU/ml of penicillin and 100 μg/ml of streptomycin. The IMR32 cells are cultured in the same medium but are seeded in an amount of 2 10⁴ cells/cm². NXS2 cells, seeded in an amount of 2 10⁴ cells/cm² are kept in DMEM medium containing 4.5 g/l of glucose (Sigma), 10% inactivated FCS, 2 mM of L-glutamine, 100 IU/ml of penicillin and 100 μg/ml of streptomycin. T84 human colon adenocarcinoma cells were provided by Dr. M. Neunlist (INSERM UMR913, Nantes). They are seeded at a density of 2 10⁵ cells/cm² and cultured in DMEM/F12 (1:1, Gibco-BRL) with 10% inactivated FCS, 2 mM of L-glutamine, 100 IU/ml of penicillin and 100 μg/ml of streptomycin.

Reagents Used

Mouse 1A4 IgM monoclonal antibody directed against neutral glycolipid Gb3 was graciously provided to us as ascites by Dr. J. Wiels (Institut Gustave Roussy, Villejuif, France). It was purified by affinity chromatography using a mannan column (Pierce, Rockford, USA) and conditioned at 4° C. in PBS. Rat anti-Gb3 IgM monoclonal antibody 38.13 and the mouse isotype control (clone 11E10) were obtained in the purified form from Beckman Coulter (Fullerton, Calif., USA). The isotype control antibody was conditioned in a 100 mM borate solution, dialyzed in PBS and then filtered on a 0.22 μm membrane. Goat biotinylated secondary antibodies such as anti-mouse IgG+IgM (H+L) antibodies, anti-mouse IgG (H+L) antibodies and anti-mouse μ antibodies were provided by Jackson Immunoresearch (Laboratories, West Grove, Pa., USA). Goat antibodies coupled to peroxidase specific for the rat μ chain and goat biotinylated secondary antibodies specific for mouse subclasses (Fc_{γ1}, Fc_{γ}2a, Fc_{γ}2b, Fc_{γ}3) came from the same provider. Goat biotinylated antibodies specific for mouse light chains (κ and λ) were provided by SouthernBiotech (Birmingham, Ala., USA). These antibodies were adsorbed against human, bovine or rabbit serum proteins. The streptavidin-R-phycoerythrin-(R-PE) complex was obtained from Biosource (Camarillo, Calif., USA), the streptavidin-horseradish peroxidase complex and the streptavidin-FITC complex were obtained from Beckman Coulter.

The mixture of neutral glycosphingolipids containing lactosylceramide (LacCer), galactosylceramide (GalCer), trihexoside ceramide (Gb3) and globoside (Gb4) as well as purified Gb3 were obtained from Matreya (Pleasant Gap, Pa., USA). These glycolipids are of either pig (Gb3 and Gb4) or cow (LacCer and GalCer) origin. Purified Gb3 was redissolved with 1 ml of a chloroform methanol mixture: (2:1 v/v) to obtain a final concentration of 1 μg/μl. Sphingosine-1-phosphate (S1P) was obtained from Biomol International (Plymouth Meeting, Pa., USA) and rehydrated (Morita et al. 2000) to obtain a concentration of 10 μM in PET diluent made up of 5% polyethylene glycol, 2.5% ethanol and 0.8% Tween-80. The mixture of standard ganglioside markers (GM₁, GD₁ₐ, GD_{1b}, GT_{1b}) was prepared in the laboratory by extraction of gangliosides from rat brains according to the technique described by Folch (Folch et al. 1951).

1.2 Extraction and Purification of Glycolipids.

Extraction of Total Glycolipids from Cell Pellets.

Glycolipids were extracted from cell pellets according to the method of S. Ladish (Ladish and Li, 2000). The cells are trypsinized, washed twice in pH 7.3 PBS, then incubated at 20° C. overnight. They then undergo hypotonic lysis in 1 ml of distilled water. Aliquots are then harvested and centrifuged at 15,000 g, 4° C., for 15 min to eliminate insoluble particles, cell debris and DNA. The protein concentration of the aliquots is then determined by absorbance at 280 nm (Nanodrop ND-1000, Labtech, Palaiseau, France) to deposit, on a thin layer of silica, a glycolipid quantity of 0.5-2.5 mg protein equivalent per sample. Lipids are extracted from lysed cells in distilled water by chloroform:methanol (1:1, v/v; 20 ml/10⁹ cells) for 18 hours with mechanical stirring at room temperature. The samples are then centrifuged for 10 min at 750 g and the cell pellets extracted again for 4 h in an additional volume of chloroform:methanol (1:1, v/v). After evaporation of the organic solvent containing the glycolipids under nitrogen flow, the total lipid extract is obtained.

Purification of Glycolipids.

First Step: DIPE/1-Butanol Partition.

The total lipid extract is redissolved in 5 ml of an organic mixture containing DIPE/1-butanol (60:40, v/v). Series of vortexing and sonication (Diagenode's Bioruptor, Sparta, USA) are alternated and repeated until an opalescent solution is obtained. By adding and mixing 2.5 ml of saline solution at 0.1% NaCl, a partition is obtained after centrifugation at 750 g for 10 min at 4° C. The upper organic phase containing neutral lipids and phospholipids is collected with the pipette and may be stored at −20° C. The lower aqueous phase and the interface containing the most hydrophilic acid glycolipids and neutral glycolipids are partitioned again with a new volume of DIPE/1-butanol (60:40, v/v). The glycolipids contained in the aqueous phase are evaporated in a speed-vac (Eppendorf, Hamburg, Germany).

Second Step: Desalting by Gel Filtration

In order to eliminate salts and contaminants such as proteins that may be co-extracted at this stage, the dry residues are redissolved and vortexed in 500 μl of solvent A (chloroform:methanol:H₂O (30:60:8, v/v/v)) then introduced into a LH-20 Sephadex 10-ml column, 1×30 cm (Pharmacia Fine Chemicals, Uppsala, Sweden) pre-equilibrated with solvent A. After adding the glycolipids to desalt, the first 3 ml of solvent A are eluted then the next 3 ml are collected as the desalted glycolipid fraction. This fraction is then evaporated under nitrogen flow and stored at −20° C.

1.3 Analysis of Glycolipids by Silica Thin Layer Chromatography (HPTLC).

The recovered glycolipids are taken up in a small volume of chloroform:methanol (1:2, v/v) so as to deposit, using a TLC ATS4 automatic sampler (Camag, Muttenz, Switzerland) 2 to 2.5 mg protein equivalent per glycolipid sample. The glycolipids are separated on HPTLC plates made up of silica gel 60 covering an aluminum sheet (Merck, Darmstadt, Germany). Before depositing, these silica plates are subjected to a first migration in a glass tank (Camag) containing a mixture of chloroform:methanol (1:1, v/v), in order to remove any contaminants. After glycolipid deposit, a first migration system made up of chloroform:methanol (2:1, v/v) solvent permits migrating and eliminating very nonpolar lipids that can be co-extracted and that may hinder the separation of the glycolipids of interest. These are then separated during a final development in a saturated tank for 4 h with 70 ml of aqueous chloroform:methanol:0.2% CaCl₂ (55:45:10, v/v/v). The bands are chemically revealed at 150° C. after spraying the plate with a solution of orcinol previously prepared by dissolution of 0.2 g of orcinol (Sigma-Aldrich, Saint-Louis, USA) in 40 ml of distilled water and 10 ml of sulfuric acid. This reagent reveals the purple bands characteristic of the presence of glycosylated residues. The glycolipids can then be quantified by ImageQuant 5.2 densitometry software (GE Healthcare, Waukesha, USA).

1.4 Immunofixation of Glycolipids Separated by HPTLC (iTLC).

The antibody specificity can be determined on glycolipids separated by HPTLC. After separation of glycolipids by chromatography, for which 2 to 2.5 mg of protein equivalent are deposited per sample revealed with orcinol and 0.5 mg of protein equivalent for the samples that are stained by antibodies, the HPTLC plates are cut into strips and plasticized by incubation for 1 min in 0.1% poly-isobultyl-methacrylate dissolved in hexane, then saturated at ambient temperature for 1 hour with 1% PBS-BSA. The strips are then incubated overnight at 4° C. with the antibodies (either directly incubated with the hybridoma supernatants or incubated with 5 μg/ml of purified antibodies diluted in 0.1% PBS-BSA). After three washes with PBS, antibody binding is detected by two successive incubation steps, a first incubation step for 1 h at room temperature of biotinylated secondary antibodies (diluted to 1:2000 in 0.1% PBS-BSA), followed by incubation of the streptavidin-horseradish peroxidase complex diluted to 1:2000 for 1 h at room temperature. After washing several times with PBS, the bound peroxidase is revealed by a 4-chloro-1-naphthol solution (Sigma) which is prepared extemporaneously in an amount of 1 mg of product dissolved in 1 ml methanol, taken up in 20 ml of PBS and supplemented with 30 μl of 30 parts hydrogen peroxide.

1.5 Analysis of Antibody Specificity by Immunoenzymatic Assay (ELISA) on Desiccated Cells on 96-Well Microtiter Plates.

The desiccated cell plates were prepared as follows. The cells are trypsinized and washed three times with cold PBS, then diluted in order to obtain a cellular concentration of 2 10⁶ cells/ml. Then 50 μl of this suspension are deposited at the bottom of each of 96 wells of an Immuno-Plate microtiter plate (Nunc, Maxisorp®, Denmark). After desiccation of the cells overnight in a 37° C. oven, the plates are either used immediately or stored at room temperature for several months in aluminum.

After washing desiccated cells with PBS and blocking the non-specific interaction sites with 1% PBS-BSA for 1 h at room temperature with slow stirring, antibodies diluted in 0.1% PBS-BSA (10 μg/ml of the initial concentration) are deposited in each of the wells in triplicate, at a volume of 100 μl of antibodies according to a dilution range of 1:256. The plates are incubated for 2 h at room temperature then washed three times with PBS. Antibody binding is successively detected by a first incubation for 1 h at room temperature with biotinylated secondary antibodies (diluted to 1:4000 in 0.1% PBS-BSA), then after washing, by incubation of the streptavidin-horseradish peroxidase complex (diluted to 1:4000, 1 h at room temperature). After several washes with PBS, peroxidase binding is revealed by an ABTS solution (Merck) which shows a progressive green color. The reaction can then be blocked by addition of 10% SDS. The optical density is measured by reading the plate with a spectrophotometer at 405 nm (Multiskan Thermo Electron reader, Illkirch, France).

1.6 Study of Cell Staining by Immunofluorescence and Flow Cytometry.

To detect cell surface antigens by flow cytometry, cells are trypsinized and washed twice in cold PBS containing 2.5% decomplemented FCS (PBSF). They are then taken up in PBSF and deposited in an amount of 4 10⁵ cells per well on a conical bottom well (Nunc, Denmark). The plate is centrifuged at 750 g for 1 min in order to remove the supernatant then the non-specific binding sites are blocked by an incubation of 30 min directly on ice with 5% human serum, prefiltered (0.22 μm) and decomplemented at 56° C. for 45 min (donated by EFS, Nantes). The cells are then directly incubated with 100 μl of primary antibodies diluted to 10 μg/ml in PBSF for 45 min on ice. After incubation, the cells are washed three times with cold PBS, and then fixed in 4% paraformaldehyde (Electron Microscopy Science, Washington, USA) in PBS, on ice for 15 min, in order to prevent antigen-antibody complexes from being internalized. After three washes with PBS, antibody binding is detected first by an incubation of 30 min on ice for biotinylated secondary antibodies (diluted to 1:400 in PB SF) followed by washes and then incubation of the streptavidin-phycoerythrin complex (diluted to 1:400, 30 min on ice). After several washes with PBS, the distribution and intensity of cell fluorescence (1 10⁵ cells) is determined by means of a FACSCalibur flow cytometer and Cell Quest Pro execution software (BD Biosciences, San José, Calif., United States).

1.7 Cellular Localization of Gb3 by Immunocytochemistry

Cells are pre-seeded in the culture medium on 14-mm diameter glass slides (Thermo Scientific, Hudson, USA) in a 24-well plate, 24 to 48 h before staining, in an amount of 2 10⁵ cells/cm². The non-specific binding sites are blocked by incubation on ice of 5% deactivated human serum and then the cells are incubated with antibodies diluted to 40 μg/ml in PBSF for 45 min on ice. After staining, the cells are washed 3 times with cold PBS, and then fixed with 4% paraformaldehyde on ice for 15 min. The cells are again washed three times with PBS and antibody binding is detected first by an incubation of 30 min on ice for biotinylated secondary antibodies (diluted to 1:400 in PBSF) followed by 30 min of incubation on ice of the streptavidin-FITC complex (diluted to 1:400). After staining of the cell membrane, the nuclei are stained for 15 to 20 min at room temperature with Drag5 (Biostatus, Leicestershire, United Kingdom) diluted to 1:1000 in PBS. The slides are mounted in Fluoromount-G medium (SouthernBiotech, Birmingham, Ala., USA) and cell staining is observed with a TCS-SP1 confocal microscope (Leica, Mannheim, Germany, PICell platform, IFR 26, INSERM, Nantes) at a magnification of 63×.

1.8 Analysis of the Distribution of Gb3 by Immunohistochemistry on Frozen Tumor Sections.

Mice are reared in the animal room of INSERM unit U892 (under the control of the association Française des Sciences and Techniques de l'Animal de Laboratoire (AFSTAL, the French Association of Laboratory Animal Science and Techniques).

Raji cells and IMR32 cells (2.5 10⁵ cells) diluted to 1:2 in Matrigel with a high concentration of growth factors (BD Biosciences, Bedford, USA) are injected subcutaneously into the flanks of 12-week old Balb/c@BYJ Rj mice (Janvier, St Berthevin, France). The mice were sacrificed by neck stretching as soon as small blood capillaries appeared on the xenograft tumors. Directly after excision, the tumors were pre-immersed in isopentane and then immersed for several seconds in liquid nitrogen to be stored at −80° C. Frozen 5 μm sections made with the Leica CM-1900 cryostat (IFR 26 morphology platform, INSERM, Nantes) are deposited on Starfrost slides (Knittel Glaser, Braunschweig, Germany). They are then treated by reheating to room temperature for 30 min, fixed in cold acetone for 10 min, air-dried for 30 min and washed in PBS, before proceeding to the staining procedure. First, the non-specific binding sites are blocked with Kit MOM blocking reagent (Vector Laboratories, Burlingame, Calif., USA). The frozen sections are incubated successively with 50 μg/ml of anti-Gb3 antibodies or mouse IgM isotype control antibodies (clone 11E10) for 1 h at room temperature, then incubated after several washes with PBS, with the goat anti-mouse IgM biotinylated secondary antibodies, diluted to 1:100. Detection is done with a chromogenic substrate. The sections are incubated for 30 min with VECTASTAIN® Elite ABC reagent (Vector Laboratories). Peroxidase activity is detected with the DAB® Menarini Kit substrate (Rungis, France) diluted to 1:50, which leads to the deposit of a brown pigment. The sections are then counterstained with hematoxylin and observed at a magnification of 40× with a DM IRB microscope (Leica).

1.9 Obtaining 3E2 (IgM, κ) Anti-Gb3 Antibodies.

Immunization in Mice and Somatic Hybridization Protocol.

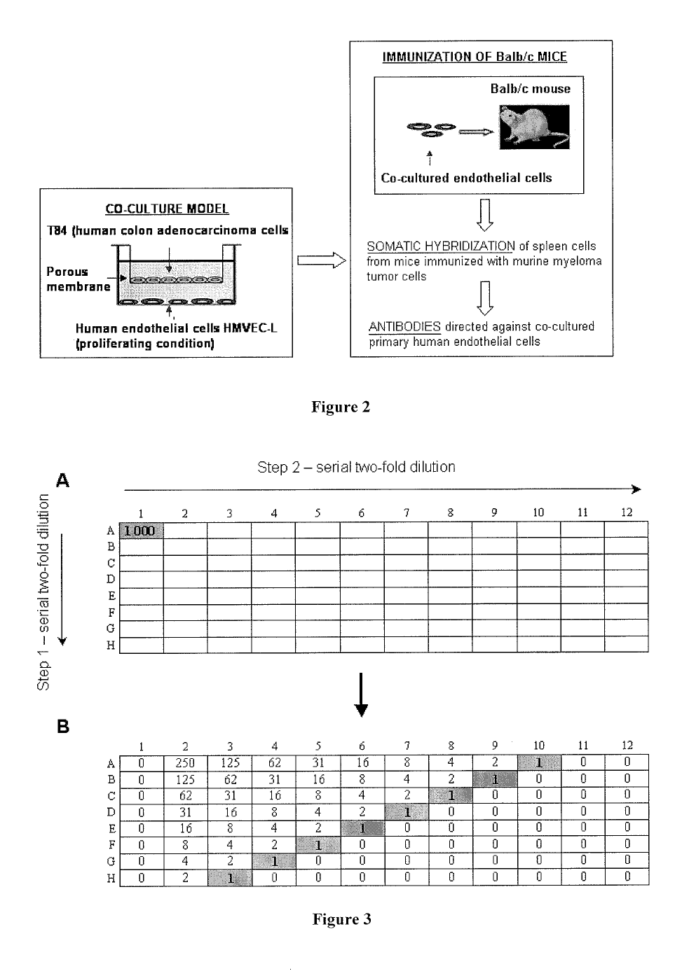

Five 6-week old Balb/c@BYJ Rj mice were immunized with HMVEC-L cells previously co-cultured with T84 human colon adenocarcinoma cells by means of membranes allowing the exchange of soluble factors between the two cellular compartments (Gaugler et al. 2007). The HMVEC-L cells were then seeded on 6-well plates in an amount of 5 10⁴ cells/cm² in EGM®-2 MV culture medium. Three days later, while the endothelial cells were still proliferating, T84 cells (1 10⁵ cells/cm²) were seeded in their DMEM:F12 culture medium, on the porous filtering membrane (0.4 mm pores) of a Transwell 6-well Transwell-Clear insert (Corning, the Netherlands) for 72 hours. The HMVEC-L were then trypsinized, washed in in PBS, fixed with 4% paraformaldehyde and stored at 4° C. in incomplete Freund's adjuvant (Sigma, Saint-Louis, USA). In all, the mice received five injections at weeks 0, 8, 10, 32, and 60 of 2.1-2.8 10⁶ previously co-cultured HMVEC-L cells and a booster at 65 weeks of 1.5 10⁶ cells (preserved in PBS without adjuvant), 6 days before their sacrifice. The mouse humeral response kinetics were analyzed during the immunization period by ELISA tests performed on desiccated HMVEC-L cells as well as on desiccated HMEC-1 cells in order to consider the use of these transformed cells for antibody screening studies. As soon as there is a positive anti-antibody response, the mouse splenocytes were fused with mouse SP2/O myeloma cells in a fusion ratio of 5/1:2, 5 10⁸ splenocytes were fused with polyethylene glycol (PEG 1500, Sigma) at 0.5 10⁸ cells of mouse SP2/O myeloma cells (cultured in RPMI 1640). After fusion, the cells were taken up in 200 ml of RPMI 1640 medium supplemented with 20% inactivated FCS, 2 mM L-glutamine, hypoxanthine-aminopterin-thymidine HAT 1× medium (Sigma-Aldrich) and seeded in an amount of 100 μl in 35 96-well microplates. At the end of 15 days, the first hybridomas were visible and could be amplified in 12-well microplates in a RPMI 1640 medium supplemented with 20% deactivated serum, 2 mM L-glutamine, 100 IU/ml penicillin and 100 μg/ml streptomycin. The hybridoma supernatants were harvested for screening and stored at 4° C. and all the hybridomas were taken up in FCS containing 10% dimethyl sulfoxide, to be stored at −80° C. The antibody generation diagram is recapitulated in FIG. 2.

Antibodies Cloning and Purification.

Antibodies Cloning.

Selected hybridomas were cloned by the limiting dilution method (FIG. 3). Each clone obtained was again tested by ELISA using anti IgG+IgM detection antibodies, then the isotype of the positive monoclonal antibodies was determined using anti-isotype (μ, γ1, γ2a, γ2b and γ3) and anti-light chain (κ and λ) detection antibodies.

Purification of Antibodies by Affinity Chromatography

Selected antibodies were then purified according to the supplier protocol using a 5 ml protein L column (Pierce, Rockford, USA) with an affinity for immunoglobulin kappa light chains. The hybridoma supernatant was diluted with a 0.1 M Tris-HCl solution, pH 7.8 (dilution 1:1), filtered on 0.22 μm and then slowly passed over the column. Non-specifically bound proteins were eliminated by 5 volumes of PBS. The antibodies are collected by elution with a 0.1 M glycine-HCl buffer solution, pH 2.5 and the eluate is neutralized with a 0.1 M Tris buffer solution, pH 9. The eluates are selected after measurement of absorbance at 280 nm (Nanodrop) and grouped to be dialyzed in PBS, filtered on 0.22 μm and stored at 4° C.

The degree of antibody purity is assessed by electrophoretic analysis under denaturing conditions (SDS-PAGE) and either reducing conditions with 12% acrylamide (375 mM of pH 8.8 Tris, 0.1% SDS, 0.1% APS, TEMED), or non-reducing conditions with 6% acrylamide. Gel migration is done with 5 μg of antibodies per condition, at 90 V for 1 hour and 45 min in Tris-glycine-SDS buffer (Biorad, Hercules, USA). The electrophoretic profile is detected by Coomassie blue staining.

1.10 Determination of Affinity Constants for Anti-Gb3 Antibody 3E2.

The antibodies are first marked with iodine 125 in the presence of an oxidant, iodogen. In a glass tube whose walls were treated with 10 μg iodogen in a volume of 100 μl, 200 μg of anti-Gb3 antibody 3E2 are deposited, then the tube is filled with 100 μl of 0.1 M phosphate buffer, pH 7.2. Iodine 125 (Perkin Elmer, Waltham, Mass., USA) is added so as to obtain an activity of 1 μCi/μg of antibody, or 200 μCi of activity. The reaction mixture is incubated for 30 min with slow stirring at room temperature. After incubation, the purity of the radiolabeled antibodies was checked by thin-layer chromatography. For this, 2 μl of the reaction medium were deposited on an ITLC-SG strip (PALL Science, Iris Parkway, USA). During migration in 10% trichloroacetic acid, the free I¹²⁵ is separated from the I¹²⁵ bound to antibodies. Then the amount of stained antibodies can be evaluated, which must be greater than 90% and which can be improved by purification on a NAP-5™ column (GE Healthcare, Waukesha, USA) and again evaluated by ITLC-SG thin layer chromatography.

Once antibody 3E2 is radiolabeled, the number of Gb3 antigenic sites was determined according to the Scatchard technique on Raji lymphoma lines and on HMEC-1 endothelial cells. For HMEC-1 cells, the number of antigenic sites was evaluated after 24 h of incubation in depleted medium (MCDB 131 medium supplemented with 0.1% decomplemented FCS, 2 mg/ml of hydrocortisone, 2 mM of L-glutamine, 100 IU/ml of penicillin and 100 mg/ml of streptomycin) and after 24 h of incubation in complete medium (MCDB 131 medium supplemented with 15% decomplemented FCS, 10 ng/ml of EGF, 2 mg/ml of hydrocortisone, 2 mM of L-glutamine, 100 IU/ml of penicillin and 100 mg/ml streptomycin) In brief, in a 96-well plate with a round bottom 2.5 10⁵ cells were contacted with antibodies previously radiolabeled and serially two-fold diluted, from 555 nM to 0.13 nM (n=3). At the same time, wells containing a mixture of 500 nM cold antibodies with 5 nM radiolabeled antibodies, two-fold diluted four times, are used to determine non-specific binding. The reaction medium is then incubated at 4° C. with stirring for 1 h, then 50 μl of this mixture were added into Scatchard tubes (Sarstedt, Numbrecht, Germany) containing 200 μl of a separation solution composed of 10% paraffin and 90% dibutyl phthalate. After centrifugation at 15,000 g for 3 minutes, the tubes are immersed in liquid nitrogen and divided into two parts to be immediately placed in hemolysis tubes for radioactive counting. The lower end containing the cells is the bound fraction and the upper end containing the supernatant is the unbound fraction. The measurements were done with a gamma counter (1480 Wizard 3 counter, Perkin Elmer, Finland) and the results were analyzed with GraphPad Prism software (GraphPad Software Inc., San Diego, USA).

1.11 Biological Properties of Antibody 3E2

MTT Cell Viability Test.

The MTT test (Mosmann, 1983) is based on the transformation of MTT (3-(4,5-dimethyl-2-thiazolyl)-2,5-diphenyl-2H-tetrazolium bromide) into blue formazan crystals by a mitochondrial enzyme, succinate dehydrogenase. The formazan crystals formed are chemically solubilized and detected by spectrophotometry at a wavelength of 570 nm. This test is used to compare the viability of control cells to that of cells treated by the molecules.

Briefly, the test is done on 24-well plates with cells seeded at a density of 2 10⁴ cells/cm². Adherent cells like HMEC-1 and NXS2 cells are incubated, 12 hours after seeding, with primary antibodies previously diluted in 300 μl of complete medium to give a final concentration of 100, 60, 40, 20, 10 and 0 μg/ml (n=3). Raji cells in suspension are directly seeded into the culture medium containing the antibodies (n=3). They are then incubated for 24 hours at 37° C., 5% CO₂. Then 500 μg/well of stock MTT solution (Roche, Mannheim, Germany) are added and the cells are kept at 37° C. for 4 hours to allow formazan crystal synthesis. 100 μl/well of the solubilization solution are added (10% SDS in 10 mM HCl). After in incubation of one night at 37° C., the plates are read with a Multiskan (Thermo Electron, Illkirch, France) reader by measuring absorbance at 570 nm and, for background noise, 650 nm

Video-Kinetic Study

HMEC-1 cells were then seeded in a 24-well microplate at an amount of 2 10⁵ cells/cm² and then kept for 12 hours in their complete medium at 37° C., 5% CO₂. The 3E2 and isotype control antibodies are then diluted to 20 μg/ml in complete culture medium and incubated with the cells, in triplicate. Immediately after incubation, digital images were record for 24 to 72 h to produce an accelerated film (Leica, DMI6000B/PICell platform). Observations were made of the number of cells per time interval, the number of cells in division per time interval and the cell division time (mean of the observations of three different wells).

Aortic Ring Tests

Four-week old C57bl6 mice were provided by the breeder Janvier (St Berthevin, France). Abdominal aorta were isolated when the mice were 6 to 8 weeks old. They were then cleaned in MCDB 131 complete medium and cut into cross sections of 0.5 to 1 mm width. These rings were then deposited at the bottom of a 96-well microplate pretreated with 30 μl of Matrigel (ECM Gel, Sigma-Aldrich, Saint-Louis, USA) two-fold diluted in PBS. The rings were then covered with 20 μl of non-diluted Matrigel then kept for 5 days at 37° C., 5% CO₂ in a complete medium containing 0, 20 and 40 μg/ml of antibodies. The culture medium with or without antibodies was renewed every 2 days. At the end of 5 days of incubation, the formation of vascular buds was observed by microscopy (DM IRB Microscope, Leica). To establish an objective measurement of the formation of these buds, we asked a panel of five people to participate independently in grading blind images of aortic rings recorded at 10× magnification and we asked them to assign to each image a score based on the number, length and density of microvessels formed (scale of 0 to 5, the minimum value of 0 corresponding to the absence of buds) to assess the antiangiogenic activity of the antibodies.

Evaluation of Cytotoxic Activity in the Presence of Complement.

The CDC activity is measured by flow cytometry by means of an iodide propidium (IP) solution that can incorporate cell DNA. The cells are trypsinized, taken up in the culture medium, then deposited in 96-well plates with conical bottoms (Nunc, Denmark) in an amount of 0.2 10⁶ cells/well. The plates are then centrifuged in order to eliminate the supernatant, and the cells are incubated with the antibody to give a final concentration of 0.1 to 10 μg/ml. Fresh filtered serum is collected 24 hours after the blood sample in the absence of anticoagulant. A fraction is decomplemented at 56° C. for 45 min, filtered again and stored at 4° C. Then human serum is added that represents 1:5 of the final volume, either in its decomplemented form or in its non-decomplemented form. The plates containing the cells, antibodies and serum are then incubated for 2 h at 37° C., 5% CO₂. The cell pellets are then collected by brief centrifugation of the plate and the cells taken up with 0.6 μg of a propidium iodide solution in a volume of 100 μl. After an incubation of 30 min at 37° C. protected from light, the cells are directly analyzed by means of cytometry in an amount of 1·10⁵ cells per analysis.

1.12 Nucleotide Sequencing of the Variable Regions of the Heavy and Light Chains of Anti-Gb3 Antibodies.

Extraction of Total RNA.

Total RNA of mouse hybridomas was extracted according to the method described by Chomczynski and Sacchi (Chomczynski and Sacchi, 1987) with RNAble® reagent (Eurobio, Les Ulis, France). The cells are homogenized in a denaturing solution containing guanidinium isothiocyanate and phenol. The nucleic acids are denatured and the proteins are dissociated by the formation of complexes between RNA and guanidine isothiocyanate, which permits breaking the hydrophilic interactions between DNA and proteins. Thus, DNA and proteins are extracted from the aqueous phase while RNA remains in this phase. The hybridoma cell pellet (10 10⁶ cells) is washed twice in PBS then taken up in 2 ml of RNAble®. The cells are lysed by repeated pipetting in order to dissociate the nucleotide-protein complexes. After addition of 0.2 ml of chloroform, the homogenate is mixed vigorously for 15-30 seconds. This step separates the nucleic acids by differential solubilization (since RNA is insoluble in phenol) from proteins. The mixture is incubated on ice for 5 min and then centrifuged for 15 min at 12,000 g at 4° C. The aqueous phase containing RNA is drawn off then transferred into a microtube. Once precipitated by 2 ml of isopropanol for 10 min at room temperature, they are recovered by centrifugation at 12,000 g for 5 min at 4° C., then washed twice with 1 ml of a solution of 75% ethanol (v/v), air-dried and taken up in 15 μl of 0.1% DEPC-H₂O (water pre-treated against ribonucleases by diethyl pyrocarbonate). The RNA concentration was determined spectrophotometrically by measuring the absorbance at 260 nm of an aliquot and its purity was determined at 280 nm by the ratio of absorbance at 260 nm/280 nm for a value of about 2. The RNA thus extracted is either stored at −80° C. or directly treated with DNAse.

Treatment with DNAse 1.

In order to eliminate any trace of genomic DNA, the following DNAse treatment is conducted following the supplier protocol. The reaction is conducted in a final volume of 10 μl comprising 2 μg of total RNA, a 20 mM Tris-HCl reaction buffer solution, pH 8.4, 2 mM MgCl₂, 50 mM KCl, and 1 U of DNAse 1 (Sigma, St-Quentin-Favier, France). After an incubation of 15 min at ambient temperature, the DNAse is deactivated by the addition of 1 μl of a 25 mM EDTA solution, followed by incubation at 65° C. for 10 min. The treated RNA may also be stored at −80° C., or even be directly subjected to a reverse transcription step.

Synthesis of Complementary DNA.

Retrotranscription followed by polymerization stabilizes single strand RNA into complementary DNA (cDNA). cDNA is synthesized from 0.2 μg of RNA in a final volume of 40 μl. According to the supplier protocol (Invitrogen), the total RNA is denatured by incubation at 65° C. for 5 min in the presence of 500 ng poly-(dT)₁₂₋₁₈ oligonucleotides and 1 nM of each dNTP in 12 μl d′H₂OmQ qsp. After incubation, the RNA is quickly placed on ice to prevent folding of its secondary structures, then 14 μl of reaction medium are added composed of reaction buffer (250 mM Tris-HCl, pH 8.3, 375 mM KCl, 15 mM MgCl₂), 0.2 μM DTT, 40 U of RNaseOUT® and 200 U of M-MLV (Moloney Murine Leukemia Virus Reverse Transcriptase). After 50 min of incubation at 37° C., the reaction is stopped by 15 min pf incubation at 70° C. The DNA thus obtained is stored at −20° C. or directly amplified by PCR.

Amplification of V_{H} and V_{L} DNA by PCR.

Gene amplification by PCR of V_{H} and V_{L} DNA is done by means of the specific oligonucleotide pairs described in Table 4 (Clackson et al. 1991, Lefranc and Lefranc, 1997). From the 5′ side, the oligonucleotides hybridize to the FR1 region and from the 3′ side, to the various J_{H} or J_{L} segments.

TABLE 4
Primers used for amplification of the variable regions of the V_{H} and V_{L}
gene segments of mouse monoclonal antibodies.
A - VH Primers
VH back primers (22-mer)

| | |
|---|---|
| VH1 A back | 5′ - AGG TGC AGC TTC AGG AGT CAG G - 3′ SEQ ID NO: 71 |
| VH1 B back | 5′ - AGG TGC AGC TAG AGG AGT CAG G - 3′ SEQ ID NO: 72 |
| VH2 A back | 5′ - AGG TCC AGC TGC AGC AGT CAT G - 3′ SEQ ID NO: 73 |
| VH2 B back | 5′ - AGG TCC ACC TGC AGC AGC CTG G - 3′ SEQ ID NO: 74 |
| VH2 C back | 5′ - AGG TCC AGC TGC AGC AGT CTG G - 3′ SEQ ID NO: 75 |
| VH3 A back | 5′ - AGG TGA AGC TGG TGG AGT CTG G - 3′ SEQ ID NO: 76 |
| VH3 B back | 5′ - AGG TGA AGC TTC TCG AGT CTG G - 3′ SEQ ID NO: 77 |
| VH3 C back | 5′ - AAG TGA AGC TTG AGG AGT CTG G - 3′ SEQ ID NO: 78 |
| VH3 D back | 5′ - AAG TGA TGC TGG TGG AGT CGT G - 3′ SEQ ID NO: 79 |
| VH5 back | 5′ - AGG TTC AGC TCC AGC AGT CTG G - 3′ SEQ ID NO: 80 |

JH for primers (24-mer)

| | |
|---|---|
| JH1 for | 5′ - TGA GGA GAC GGT GAC CGT GGT CCC - 3′ SEQ ID NO: 81 |
| JH2 for | 5′ - TGA GGA GAC TGT GAG AGT GGT GCC - 3′ SEQ ID NO: 82 |
| JH3 for | 5′ - TGC AGA GAC AGT GAC CAG AGT CCC - 3′ SEQ ID NO: 83 |
| JH4 for | 5′ - TGA GGA GAC GGT GAC TGA GGT TCC - 3′ SEQ ID NO: 84 |

B - Vκ primers
Vκ back primers (24-mer)

| | |
|---|---|
| VκA back | 5′ - GAT GTT TTG ATG ACC CAA ACT CCA - 3′ SEQ ID NO: 85 |
| VκB back | 5′ - GAT ATT GTG ATA ACC CAG GAT GAA - 3′ SEQ ID NO: 86 |
| VκC back | 5′ - GAC ATT GTG CTA/G ACC CAA/G TCT CCA - 3′ SEQ ID NO: 87 |
| VκD back | 5′ - GAC ATC CAG ATG AC(N) CAG TCT CCA - 3′ SEQ ID NO: 88 |
| VκE back | 5′ - CAA ATT GTT CTC ACC CAG TCT CCA - 3′ SEQ ID NO: 89 |
| VκF back | 5′ - GAA AAT GTG CTC ACC CAG TCT CCA - 3′ SEQ ID NO: 90 |

Jκ for primers (24-mer)

| | |
|---|---|
| Jκ1 for | 5′ - CCG TTT GAT CAG CTT GGT GCC - 3′ SEQ ID NO: 91 |
| Jκ2 for | 5′ - CCG TTT TAT TTC CAG CTT GGT CCC - 3′ SEQ ID NO: 92 |
| Jκ3 for | 5′ - CCG TTT TAT TTC CAA CTT TGT CCC - 3′ SEQ ID NO: 93 |
| Jκ4 for | 5′ - CCG TIT CAG CTC CAG CTT GGT CCC - 3′ SEQ ID NO: 94 |

The PCR amplification conditions specific to each hybridoma are described in Table 5 (Lefranc and Lefranc, 1997).

TABLE 5
Experimental conditions for PCR amplification
of mouse V_{H} and V_{L} DNA

| | Type of | VH or Vκ back | JH or Jκ for | PCR |
|---|---|---|---|---|
| | amplification | primers | primers | conditions |
| VH | a | VH1 A, VH1 B | JH1, JH2, | 94° C., 50 s |
| | | | JH3, JH4 | 60° C., 90 s |
| | | | | 72° C., 90 s |
| | b | VH2 A, VH2 B, | JH1, JH2, | 94° C., 50 s |
| | | VH2 C, VH5 | JH3, JH4 | 60° C., 120 s |
| | | | | 72° C., 120 s |
| | c | VH3 A, VH3 B | JH1, JH2, | 94° C., 50 s |
| | | | JH3, JH4 | 65° C., 90 s |
| | | | | 72° C., 90 s |
| | d | VH3 C, VH3 D | JH1, JH2, | 94° C., 60 s |
| | | | JH3, JH4 | 55° C., 120 s |
| | | | | 72° C., 120 s |
| VL | e | VκA, VκB, | Jκ1, Jκ2, | 94° C., 90 s |
| | | VκE, VκF | Jκ3, Jκ4 | 60° C., 90 s |
| | | | | 72° C., 90 s |
| | f | VκC, VκD | Jκ1, Jκ2, | 94° C., 50 s |
| | | | Jκ3, Jκ4 | 65° C., 90 s |
| | | | | 72° C., 90 s |

Table 5 proposes four different amplification conditions for the amplification of heavy chain variable regions (a, b, c and d) and two different conditions for amplification of light chain variable regions (e and f). For each of these conditions, there are mixtures of primers that hybridize from the 5′ side (back primers) and the 3′ side (forward primers). In order to sequence the variable regions of the heavy and light chains, we used PCR first to identify the condition (a, b, c and d, e and f) that permitted optimal amplification of the V_{H} and V_{L} DNA and then to identify the back primer that permitted the best amplification.

All the PCR reactions were done in a final volume of 25 μl containing 1 μl of cDNA, each primer (10 pM), each dNTP (10 nM), MgCl₂ (62.5 nM), reaction buffer, 5 U of GoTaq® DNA polymerase (Promega, Charbonnières-les-Bains, France) and H₂OmQ 25 μl qsp. Thirty amplification cycles (Gene-Amp® PCR System 2700, Applied Biosystems, Courtaboeuf, France) were performed with a denaturation step at 94° C. for 5 min. Each of the cycles included a step of 10 min at 72° C. to terminate the synthesis of DNA strands. The amplification products were then visualized in 1% agarose gel (QA-Agarose-™, MP Biomedicals, Strasbourg, France) containing 0.1% GelRed™ (FluoProbes, Montluçon, France).

For each hybridoma, three independent RNA extractions and three independent PCR amplifications were performed to sequence the heavy and light chain variable regions three independent times. The amplification conditions chosen for each hybridoma are listed in Table 5.

Nucleotide Sequencing

Nucleotide sequencing was done by the IFR 26 “DNA Sequencing and Genotyping platform” of the University of Nantes, according to the Sanger method (Sanger et al. 1977) with a 48-capillary AB3730 sequencer (Applied Biosystems). For each sequencing, 1 μl of appropriate back primer (Table 6) and 5 μl of V_{H} or V_{L} PCR product were used. The PCR products were purified by the ExoSAP-IT process (Amersham GE Healthcare) which degraded single strands smaller than 100 pb with an enzyme and thus eliminated the excess nucleotide primers and dNTP.

TABLE 6
Back primers used for nucleotide sequencing.

| | |
|---|---|
| Heavy chain | Light chain |

| | PCR | Back | PCR | Back |
|---|---|---|---|---|
| Hybridoma | conditions | primer | conditions | primer |
| 3E2.c | a | VH1 A | e | VκF |
| 14C11.1d | b | VH1 A | e | VκA |
| 15C11.h | b | VH2 A | e | VκF |
| 22F6.g | b | VH2 B | e | VκF |
| 25C10.h | b | VH2 B | e | VκA |
| 11E10.h | b | VH2 C | e | VκA |
| 16G8.h | c | VH3 A | e | VκF |

The three independent sequencings conducted on three different RNA extractions allowed us to compare and determine the exact sequences of the variable regions of the heavy and light chains of each hybridoma. The nucleotide sequences obtained were then aligned with the IMGT (International Immunogenetics Information System®, http://www.imgt.org) bank in order to study the V_{H} and V_{L} gene repertoire used in mice.

### Example 2

### Looking for Glycolipid Markers of Endothelial Cells in the Process of Angiogenesis

In order to look for glycolipid markers of intra-tumor endothelial cells, we studied the glycolipid polymorphism of these cells according to different stimuli. HMEC-1 cells were transformed by SV40 virus, to make them easier to use in culture while allowing them to retain the characteristics of microvascular endothelial cells. They were then placed under different culture conditions similar to the stimuli that endothelial cells may undergo in a tumor microenvironment, which includes sending chemical signals by the tumor, such as growth factors that will induce proliferation. Then we compared the glycolipid expression profiles of proliferating or confluent HMEC-1 cells, incubated in complete or depleted medium (in serum and in growth factor) or possibly incubated with a proangiogenic factor, sphingosine-1-phosphate. The proportions of different glycolipids could then be estimated by densitometry (ImageQuant^{5.2}).

2.1 Results: Demonstration of Glycolipid Expression Polymorphism of HMEC-1 Endothelial Cells

We sought to demonstrate an expression polymorphism for endothelial cells depending on various conditions, to look for the presence of new glycolipid markers and/or the presence of overexpressed glycolipids. We then compared the glycolipid expression of HMEC-1 cells depending on their culture state (glycolipids extracted from cells in the growth phase or at confluence) and depending on their physiological state (quiescent cells in depleted culture medium and proliferative cells in complete culture medium), as well as under the effect of proangiogenic factors (sphingosine-1-phosphate). Indeed, if there are glycolipid markers for proliferating endothelial cells, these markers are targets of interest from the perspective of intra-tumor antiangiogenic therapy.

Glycolipid Expression Profile of Proliferative or Confluent HMEC-1 Cells (by HPTLC).

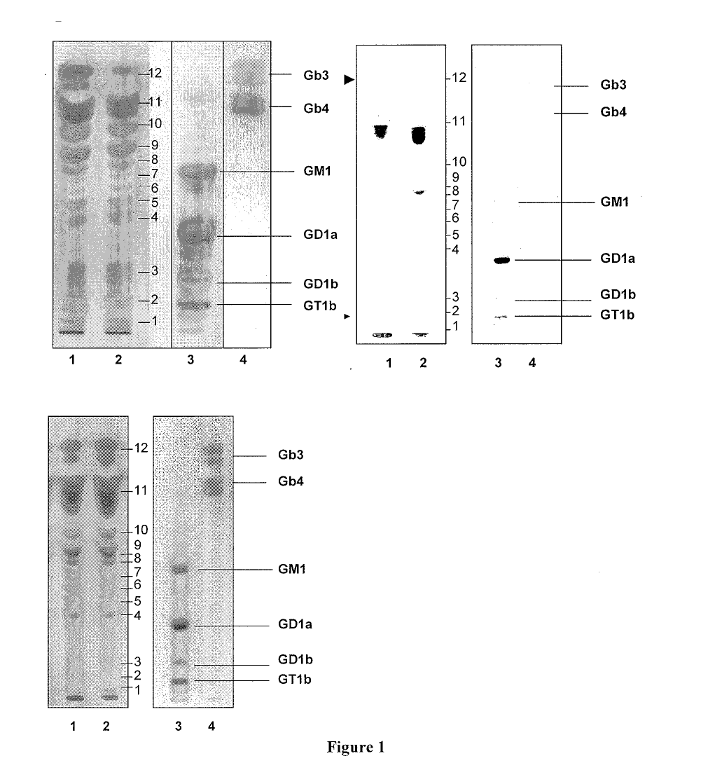

FIG. 1A shows the HPTLC analysis of glycolipids extracted when HMEC-1 cells were in the growth phase (lane 1) or at confluence (lane 2). The cells were also analyzed by densitometry.

Depending on whether the HMEC-1 endothelial cells are in the proliferation phase two or three days after seeding, or reach confluence at the end of five days, a difference in glycolipid expression is seen, especially for the most nonpolar complex gangliosides (Nos. 1 and 2) as well as glycolipid Gb3 (No. 12, FIG. 1). Densitometric analysis shows that the Gb3 content is nearly twice as high for proliferative cells as for confluent cells (equal value at 1.716±0.069, two independent analyses).

Glycolipid Expression Profile for HMEC-1 Cells According to the Composition of the Culture Medium.

FIG. 1B shows the HPTLC analysis of HMEC-1 cells that were seeded at 20,000 cells/cm² and then incubated for 24 h, as soon as the sub-confluent state is reached, in depleted culture medium (lane 1) or in complete culture medium (lane 2). The cells were also analyzed by densitometry.

Depending on whether the cells were cultured in MCDB-131 with 15% FCS (complete medium) or 0.1% FCS, without EGF (depleted medium), HPTLC shows that there is an expression difference for the most nonpolar complex gangliosides (Nos. 1, 2 and 3, FIG. 2) and for the neutral glycolipid Gb3 (No. 12). Densitometric analysis shows that the Gb3 content of cells incubated in complete medium is nearly twice as high as that for cells incubated in depleted medium (equal value at 1.512±0.061, two independent analyses).

The Glycolipid Expression Profile of HMEC-1 Cells Treated with Sphingosine-1-Phosphate for 24 h or Untreated (by HPTLC).

FIG. 1C shows the HPTLC analysis of HMEC-1 cells seeded at 20,000 cells/cm² and cultured in complete medium, until the sub-confluent state is reached, then incubated for 24 h in depleted medium supplemented with either 1 μM sphingosine-1-phosphate (lane 2), or its diluent, PET (lane 1).

This HPTLC analysis shows that there is an expression difference for Gb3 (No. 12, FIG. 1C), the Gb3 content of cells incubated 24 h with S1P is nearly 1.5 times greater than that of cells incubated with polyethylene glycol (equal value at 1.271±0.020, two independent analyses).

2.2 Conclusion

For human microvascular HMEC-1 cells transformed by SV40, we were able to develop a reproducible analysis method for glycolipids that enabled characterizing them and studying the polymorphism of glycolipid expression. The glycolipids come from the aqueous phase of a DIPE/1-butanol, 60:40 v/v, 0.1% NaCl partition and are revealed by orcinol, which allows showing 14 bands of variable intensity that would correspond to at least 7 different molecular species. We were then able to identify the presence of a neutral glycolipid that is not revealed with resorcinol/HCl, but which is recognized by rat anti-Gb3 antibody available commercially. This glycolipid Gb3 is especially present in the form of a doublet corresponding to two types of ceramide fatty acid chains.

Analysis of glycolipid expression of HMEC-1 cells as a function of different stimuli showed changes in glycolipid expression in HMEC-1 endothelial cells. An increase of the content of complex gangliosides and neutral glycolipid Gb3 is observed when the cells are in the exponential growth phase (FIG. 1A) in their complete medium (FIG. 1B), as well as an increase in the Gb3 content when they are stimulated by the presence of a pro-angiogenic factor, sphingosine-1-phosphate (FIG. 1C). The Gb3 content can be two times lower when the cells are incubated in depleted medium (0.1% FCS without EGF). In the presence of depleted medium, the cells enter the G0 phase of the cell cycle, which is a quiescent non-division stage. We verified in advance, by a cell count, that the number of HMEC-1 cells after 24 h of incubation in depleted medium was almost identical to the number of cells before incubation, whereas the number of HMEC-1 cells after incubation for 24 h in complete medium could almost double This non-division stage is reversible because under the influence of growth factors that order the cell to divide, they can again enter the G1 cell cycle to continue a normal progression of the cell cycle and proliferate.

In other words, the Gb3 content of proliferating cells is higher than that of quiescent cells. Likewise, when a proangiogenic factor like sphingosine-1-phosphate is added to depleted culture medium, the Gb3 content of the cells increases by 50%, while in complete medium, it is two times higher. We have also previously demonstrated in the laboratory, by thymidine incorporation tests, that after 24 h of incubation of HMEC-1 cells with depleted medium supplemented with 1 μM of SIP, the cell proliferation rate was 1.2 times higher compared to untreated cells, confirming the pro-angiogenic action of SP (Bonnaud et al. 2007). The greater increase in the Gb3 content in the presence of complete medium can particularly be explained by a synergistic action of the growth factors present in the fetal calf serum of the complete medium.

### Example 3

### Preparation of Antibodies Directed Against Endothelial Cells Stimulated by Tumor Cells

To generate mouse monoclonal antibodies directed against the glycolipids of intra-tumor endothelial cells, we chose to immunize Balb/c mice with endothelial cells that were previously cultured in the presence of tumor cells in a co-culture model mimicking the tumor microenvironment. In HMEC-1 endothelial cells, we showed, by HPTLC, the presence of at least seven glycolipid molecular species expressed to a greater or lesser extent, including a neutral glycolipid in the form of a doublet corresponding to glycolipid Gb3. We chose to immunize the mice with whole cells, and not with a purified glycolipid, because we did not want to favor one glycolipid in particular, or rule out the possibility of generating antibodies directed against glycoprotein antigens. Following immunization, all the hybridomas generated were frozen and all the supernatants were stored at 4° C., then we used two screening strategies to preferentially select antibodies directed against glycolipid antigens (ELISA on desiccated endothelial cells).

3.1 Results

Obtaining Antibodies

Setting Up the Co-Culture Model.

In order to generate new antibodies directed against endothelial cell glycolipid markers, we first set up a co-culture model mimicking the tumor microenvironment (FIG. 2). Primary human lung endothelial cells HMVEC-L were co-cultured in the presence of T84 human colon adenocarcinoma cells by means of porous membranes allowing the exchange of soluble factors between the two compartments (6-well Transwell-Clear, pores of 0.4 mm, Corning, the Netherlands). Once isolated and fixed with paraformaldehyde, these endothelial cells were used to immunize five Balb/c mice.

This co-culture system was developed in the laboratory and has already been described to study the influence of endothelial cell irradiation on non-irradiated T84 cells co-cultured with irradiated endothelial cells (Gaugler et al. 2007). In order to simulate the tumor microenvironment conditions as much as possible, the endothelial cells were proliferating as soon as the adenocarcinoma cells were implanted three days later and were still proliferating when they were trypsinized and fixed with 4% paraformaldehyde for immunization.

Immunization of Mice and Somatic Hybridization.

Five Balb/c mice were immunized with co-cultured endothelial cells and fixed with 4% PFA. The serums were sampled before each injection and the mouse humeral response kinetics were analyzed by ELISA on desiccated cells, both on the primary endothelial cells HMVEC-L and on HMEC-1 cells transformed by SV40. For the anti-IgM response, we used goat biotinylated polyclonal antibodies directed against the μ chain of mouse antibodies and for the anti-IgG response, goat biotinylated polyclonal antibodies directed against the (H+L) fragments of mouse antibodies.

Analysis of the immunoglobulin isotypes shows that there is an early μ response followed by a weaker γ response. In fact, for the anti-IgM response, both antibodies are detectable from the first immunization (week 8) and for the anti-IgG response, both antibodies are detectable at the end of the second injection (week 10). We also observed a cross reaction of these antibodies on HMEC-1 endothelial cells that allowed us to consider the use of these cells transformed by SV40 for screening studies.

Screening of Antibodies on HMEC-1 Cells after Somatic Hybridization

Somatic hybridization allowed obtaining 1086 hybridomas. Their supernatants were sampled for screening and stored at 4° C. and these 1086 hybridomas were taken up in FCS containing 10% dimethyl sulfoxide, to be stored at −80° C.

Due to the cross reaction observed with the antibodies present in the serum of immunized mice on HMEC-1 cells transformed by SV40 virus, we were able to consider the use of these transformed cells for screening studies. The hybridomas generated were selected by using goat detection antibodies directed against both mouse IgM (A) and IgG (H+L) and according to two approaches.

For the first approach, the hybridoma supernatants were tested by ELISA on desiccated HMEC-1 cells to preferentially target antibodies directed against glycolipid antigens. The antibodies selected by ELISA were then screened by flow cytometry since the nature of the antigens was determined by immunostaining of the HMEC-1 cell glycolipids separated by HPTLC (iTLC). For the second approach, all the antibodies preselected by ELISA were analyzed by immunostaining of HMEC-1 cell glycolipids separated by HPTLC (iTLC).

First Antibody Screening Strategy

From the 1086 hybridomas, we preselected 139 hybridomas by ELISA on desiccated HMEC-1 cells. In addition to being simple, fast, and having the advantage of selecting glycolipid motifs resistant to desiccation, this technique also does not favor a particular molecular species, since antibodies are screened on whole cells instead of purified glycolipids. The 139 hybridomas pre-selected by ELISA were then screened by flow cytometry. This method permitted selecting antibodies directed against membrane antigens at the surface of living cells. In fact, the ELISA technique can have some drawbacks. During desiccation, the cell membranes are damaged and the antibodies selected by ELISA may be directed against intracellular antigens while we are looking for antigens present at the membrane surface. By flow cytometry, we could isolate 13 hybridomas and, in order to determine the nature of the antigen recognized, we performed immunostaining of glycolipids separated by HPTLC, by these 13 hybridoma supernatants (data not shown).

Six of the supernatants of the 13 hybridomas (25C10 (1), 11E10 (3), 3E2 (4), 15C11 (5), 16G8 (10) and 22F6 (12) recognize an antigen of the same glycolipid nature present in the form of a doublet that migrates to the top of GM1, previously identified as being Gb3.

Immunostaining shows that Gb3 is present in the form of a doublet corresponding to two types of ceramide fatty acid chains. Note that certain antibodies recognize both Gb3 bands (antibody 3E2 and 15C11), others recognize the upper band (antibody 22F6) and others the lower band (antibody 25C10, 11E10 and 16G8). It has already been demonstrated that the composition of the ceramide fatty acid chain can influence the conformation of a glycolipid and thus change the reactivity of the antigen for its antibody.

In order to verify the specificity of antibodies for Gb3, we performed immunostaining of a standard mixture of neutral glycolipids including GalCer, LacCer, Gb3 and Gb4, with the supernatant of the hybridoma 3E2 antibody that has, among all anti-Gb3 antibodies, the best chromatographic profile by iTLC immunostaining and the best profile for binding by flow cytometry. The results show that this antibody recognizes only Gb3 present in the form of a doublet without having a cross reaction with globoside Gb4, or with GalCer and LacCer.

The 13 hybridomas selected were cloned by limiting dilution, again tested by ELISA on desiccated HMEC-1 cells and then we determined their isotype by a new ELISA using anti-μ, γ1, γ2a, γ2b and γ3 antibodies, and their light chain type with anti-κ and λ antibodies. The hybridomas selected by this first screening approach are listed in Table 7 below. This table shows the isotypes of each of the antibodies selected as well as the name of the corresponding monoclonal antibody.

TABLE 7
Hybridomas selected by the first screening strategy.

| Hybridoma | Isotype | mAb |
|---|---|---|
| 3E2 | IgM, κ | 3E2.c |
| 25C10 | IgM, κ | 25C10.a |
| 16G8 | IgM, κ | 16G8.h |
| 11E10 | IgM, κ | 11E10.a |
| 15C11 | IgM, κ | 15C11.h |
| 22F6 | IgM, κ | 22F6.g |

Second Antibody Screening Strategy

For the second screening strategy, we performed immunostaining on glycolipids of HMEC-1 cells separated by HPTLC with 139 hybridoma supernatants pre-selected by ELISA with anti-IgG (H+ L)+anti-IgM (μ) detection antibodies. New hybridomas (15 in all) could be selected by this second approach, notably 7 anti-Gb3 antibodies and other antibodies directed against glycolipid antigens still being characterized.

HPTLC immunostaining is a sensitive detection method that allows directly detecting purified glycolipids extracted from around 20 10⁶ to 30 10⁶ cells while whole cells in an amount of 0.4 10⁶ cells are analyzed by flow cytometry. HPTLC immunostaining allows detecting antibodies binding to minority glycolipids that might not be detectable by flow cytometry due to their low content. Moreover, at this screening stage, we are using non-cloned hybridoma supernatants whose antibody concentration is unknown. These hybridomas may only contain very small concentrations of the antibodies of interest (if the hybridoma is a poor producer or if there is a mix of hybridomas). Immunostaining on HPTLC thus allows detecting anti-Gb3 antibodies present in small quantities in the supernatants tested.

Hybridomas were cloned by limiting dilution and their isotypes were determined by ELISA using anti-isotype (μ, γ1, γ2a, γ2b and γ3) detection antibodies and detection antibodies directed against light chains (κ and λ).

In addition to the antibodies whose isotype could be determined by this technique, (such as 12E10 of isotype γ1, or 21D4 and 14C11 antibodies of isotype μ), some of them were not detectable with anti-isotype antibodies (μ, γ1, γ2a, γ2b and γ3). We assume that these antibodies may be dimers of light chains known to be secreted by myeloma cells since they can only be detected with secondary anti-IgG (H+L) antibodies and secondary antibodies specific for the κ light chain.

The hybridomas selected by this second screening approach, their isotype and the name of the corresponding monoclonal antibody are listed in Table 8 below.

TABLE 8
Antibodies selected by the second screening strategy.
Anti-Gb3

| Hybridoma | Isotype | mAb |
|---|---|---|
| 12E10 | IgG1, κ | 12E10.1C |
| 14C11 | IgM, κ | 14C11.1d |
| 15C7 | Dim, κ | 15C7.2e |
| 2F8 | Dim, κ | 2F8.1b |
| 2C8 | Dim, κ | ND |

Purification of Antibodies

In addition to the 7G10.1e antibody of the λ light chain, directed against a glycolipid in the process of being characterized and obtained during the second screening, all the antibodies of the κ light chain may potentially be purified by affinity chromatography on protein L column. The mouse monoclonal antibody 1A4 specific for Gb3, provided in the ascites form by J. Wiels, does not bind on the protein L column and was purified with a MPB mannan column (Pierce).

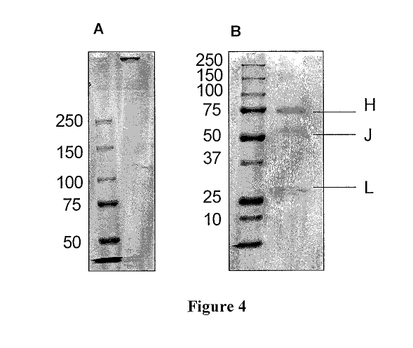

After purification, the antibody purity was analyzed by SDS-PAGE. This technique especially permits checking whether the antibodies have been denatured during the step of eluting the antibodies in the acid buffer made up of 0.1 M glycine-HCl, pH 2.5. The SDS-PAGE profile of purified antibody 3E2 is shown in FIG. 4. Antibody 3E2 is pure and undenatured. The migration of monoclonal antibody 3E2 on the polyacrylamide gel under denaturing conditions permits visualizing, under reducing conditions, three bands corresponding to the heavy chain (H) and light chain (L) and the J segment characteristic of μ immunoglobulins. Due to its high molecular weight, equal to 900,000 Da, IgM cannot migrate in 6% acrylamide gel under non-reducing conditions.

Analysis of the Specificity of Anti-Gb3 Antibodies Purified by Affinity Chromatography.

Anti-Gb3 3E2.c, 25C10.a, 16G8.h, 14C11.1d and 15C7.2e Antibodies.

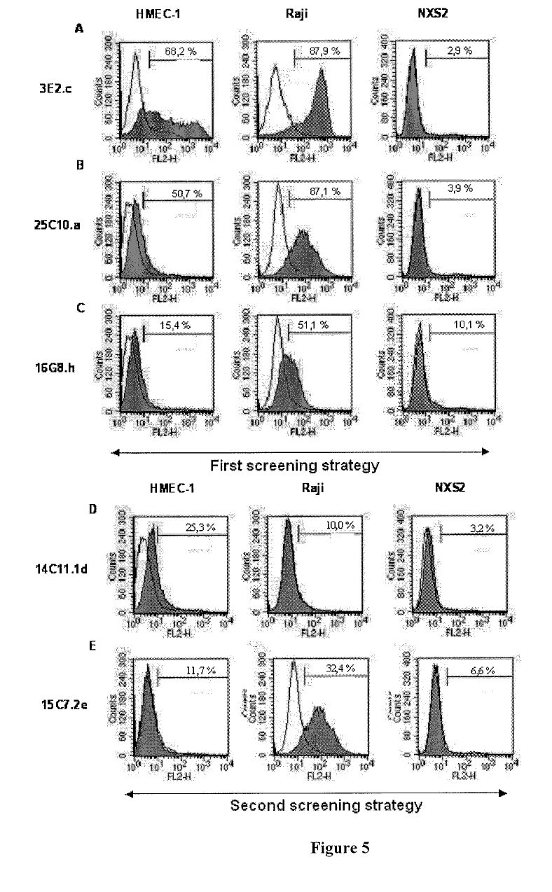

FIG. 5 shows cell staining by flow cytometry of purified anti-Gb3 monoclonal antibodies selected by the first screening strategy (3E2.c, 25C10.a, 16G8.h) or by the second screening strategy (14C11.1d and 15C7.2e). Staining was done on HMEC-1 cells, Raji lymphoma cells for which Gb3 is a marker, and mouse neuroblastoma NXS2 cells that do not express Gb3, which we had previously verified with ELISA on desiccated NXS2 cells using anti-Gb3 antibody 38.13 (data not shown). For 3E2.c, 25C10.a, 16G8.h and 14C11.1d (A, B, C and D) antibodies, we used secondary antibodies specific for the μ chain of mouse antibodies and for 15C7.2e (E) antibodies that would be a dimer of the light chain, we used secondary antibodies directed against IgG (H+L).

There is no binding on NXS2 cells regardless of the anti-Gb3 antibodies tested. The antibodies resulting from the second screening appear to have lesser affinity for Gb3 than the antibodies resulting from the first screening shown by the percentage of positive cells obtained with antibodies 14C11.1d and 15C7.2e on HMEC-1 cells (respectively 25.3 and 11.7%) and with antibody 14C11.1d on Raji cells (10.0%).

When staining is done with antibody 3E2.c, 68.2% of HMEC-1 cells and 87.9% of Raji cells are positives Unlike Raji cells, the staining of HMEC-1 cells is heterogeneous and shows that some cells strongly express Gb3 and others express it more weakly. For antibodies 25C10.a and 16G8.h from the first screening, HMEC-1 cells are more weakly stained in a homogenous manner (respectively 50.7% and 15.4%) and Raji cells are stained at a rate of 87.1% and 51.1%. Immunostaining on HPTLC of HMEC-1 cells showed that antibodies 16G8.h and 25C10.a preferentially recognized the lower band of the Gb3 doublet (data not shown), the content of which is lower than the upper band, which could explain the weak binding of these antibodies by cytometry. Binding of purified antibody 13D2.2d (data not shown) directed against a glycolipid antigen still being characterized could not be detected by flow cytometry probably due to the small quantity of this glycolipid in HMEC-1 cells or because of a poor affinity for the antibody.

Antibody 12E10.1c.

During the second screening protocol, we selected antibody 12E10.1c of isotype IgG1, which binds Gb3 of HMEC-1 cells, by immunostaining on HPTLC. Indeed, isotype IgG1 antibodies can strongly activate the complement pathway, which gives them interesting cytotoxic properties. The hybridoma was therefore cloned and purified and the antibodies were tested by flow cytometry on HMEC-1, NXS2 and Daudi cells, which are human Burkitt lymphoma cells that express Gb3. We also compared binding by flow cytometry of mAb 3E2 and 12E10.1c, on JEKO-1 human mantle cell lymphoma cells.

The results (not shown) indicate that antibody 12E10.1c hardly binds HMEC-1 cells at all and binds Daudi cells only very weakly, probably due to its weak affinity for Gb3. Moreover, it binds JEKO-1 cells more strongly, while 3E2 monoclonal antibody, having a good affinity for Gb3, does not bind these cells (data not shown). Antibody 12E10.1c no doubt recognizes a different antigen from Gb3 which is present in JEKO-1 human mantle cell lymphoma cells and has a cross reaction with Gb3 which would explain why it binds Gb3 by immunostaining on HPTLC of HMEC-1 cells.

3.2 Conclusion

Five Balb/c mice were immunized with proliferating primary HMVEC-L endothelial cells which had previously been co-cultured in the presence of tumor cells through a membrane system permitting the passage and exchange of growth factors between cells in order to mimic the tumor microenvironment. Since the immunogenicity of lipids is poor, we initially tested the serum kinetics of the antibody response by ELISA on desiccated cells for preferentially screening antibodies directed against antigens resistant to desiccation, such as glycolipids. We obtained an antibody response directed against primary HMVEC-L cells and then a cross reaction on HMEC-1 cells, which allowed considering the use of these cells transformed by SV40 as a cellular model for further studies.

This co-culture model has already been developed in the laboratory (Gaugler et al. 2007) and it has already been previously demonstrated in a similar co-culture system that the presence of tumor cells amplifies proliferation and migration of primary endothelial cells by making them acquire phenotypic and genotypic changes (Khodarev et al. 2003). The ELISA technique that uses desiccated cells was developed in the laboratory and inspired preliminary studies performed with other cell types, like screening monoclonal antibodies directed against the membrane components of human mononuclear cells. In addition, due to the fragility of pancreatic tissue, which degrades quickly and needs to be stabilized by fixation, mouse pancreatic cells were used in the desiccated form, for the detection and quantification by ELISA of antibodies present in the serum of insulin dependent diabetics. During our antibody generation strategy, we chose to immunize Balb/c mice with whole endothelial cells. Many anti-glycolipid antibodies have already been generated following immunization of whole tumor cells and in this way, new glycolipid molecular species could be identified. Initially, fixing HMVEC-L cells by 4% paraformaldehyde was chosen for screening hybridoma supernatants on cells treated under the same conditions as those that had served for immunization, but in this way too, plasma membrane HMVEC-L cells could be stabilized and the cells intended for the various immunizations during the 65 weeks could come from the same co-culture. On the other hand, since glycolipids are poorly immunogenic, they can assimilate to molecules like haptens that cannot trigger an immune reaction by themselves. To render them more immunogenic, they can be coupled to a carrier protein molecule whose role will be to expose the hapten molecule to a greater extent. By fixing glycolipids with paraformaldehyde on endothelial cell membranes, the formation of hapten-carrier complexes can be simulated in order to induce a greater immune response. In order to verify that paraformaldehyde fixation has not changed antibody binding, we performed an ELISA on desiccated HMEC-1 cells, fixed with 4% PFA or unfixed cells, before or after desiccation (data not shown). The analysis of about fifty hybridomas from 139 hybridomas selected during the first screening strategy shows that there is no change in antibody binding on desiccated cells, whether or not the cells are fixed.

Somatic hybridization permitted preselecting by ELISA on desiccated cells, from the 1086 hybridomas obtained, 139 first hybridomas that were then screened according to two strategies. The first, which consisted of screening the hybridomas on living cells by flow cytometry permitted selecting 13 of them and by immunostaining on HPTLC of glycolipid cell extracts, we determined that 6 of these recognized glycolipid Gb3. A second screening strategy, which consisted of screening by immunostaining the 139 hybridomas obtained by ELISA, permitted selecting many other antibodies, for the most part directed against Gb3 but also against glycolipids still being characterized. After purification, it was established that the antibodies generated by the second screening have a reduced affinity. Also, we chose to retain three monoclonal antibodies (IgM, κ) directed against Gb3: monoclonal antibodies 25C10 and 16G8 which, by immunostaining on HPTLC, preferentially recognize the lower band of the Gb3 doublet, which is in the minority in HMEC-1 cells and monoclonal antibody 3E2, which has the best binding profile in immunostaining on HPTLC and in flow cytometry.

Gb3 is a globotriaosylceramide, also called Gb3/CD77, or CTH (ceramide trihexoside). It was first identified as an antigen of a rare blood group, group Pk, at the surface of red blood cells and is also known as a Burkitt lymphoma marker (BLA, Burkitt lymphoma antigen). On the surface of endothelial cells, its expression is regulated by proinflammatory cytokines such as TNF-α involved in the process of tumorigenesis. Moreover, this glycolipid is known as a specific receptor for bacterial toxins and preliminary studies have shown that binding of verotoxin on Gb3 could inhibit in-vitro angiogenesis (Heath-Engel and Lingwood, 2003). These results confirm the interest of developing therapeutic antibodies targeting Gb3, especially since the content of this glycolipid seems to be modulated when endothelial cells are proliferating (see Example 2). Moreover, the great majority of anti-glycolipid antibodies generated after somatic hybridization were directed against Gb3, which would suggest that this glycolipid is fairly immunogenic in Balb/c mice. Regarding the other seven antibodies generated during the first screening strategy and whose antigens are still being characterized, it is possible that they are directed against other glycolipids of HMEC-1 cells that are not extracted by our purification by partition method (such as neutral and nonpolar glycolipids) or they may even act as glycoprotein antigens, because it is possible that glycosylated motifs carried by glycoproteins are resistant to desiccation.

### Example 4

### Characterization of Mouse Anti-Gb3 Monoclonal Antibody 3E2

4.1 Results

Determining the Affinity Constant.

Analysis of the saturation curve obtained by the Scatchard technique allowed us to evaluate the affinity constant of antibody 3E2 for the Gb3 of Raji cells and HMEC-1 cells, as well as the number of sites on their surface. The results are listed in Table 9 below.

TABLE 9
Affinity constant for antibody 3E2 for Raji and HMEC-1 cells

| | Kd (nM) | Number of sites/cell |
|---|---|---|
| HMEC-1 | 30.2 | 1.7 10⁶ |
| Raji | 29.9 | 2.0 10⁶ |

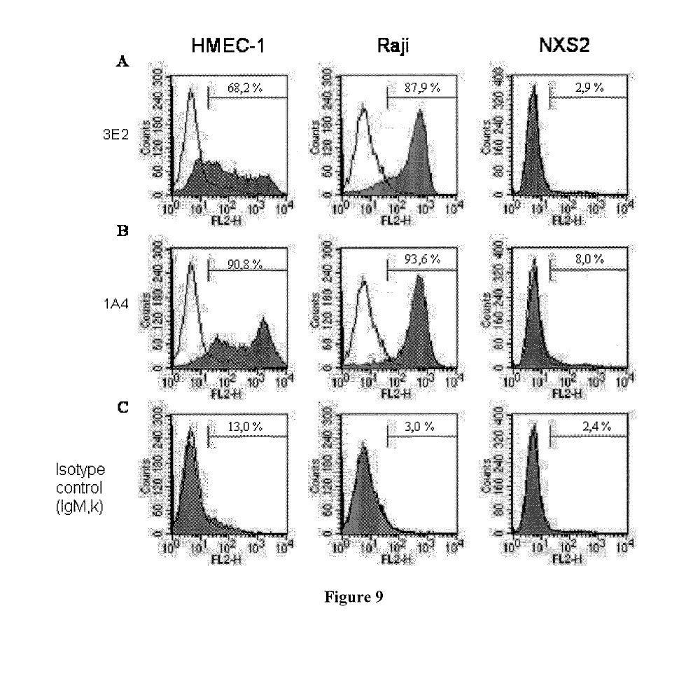

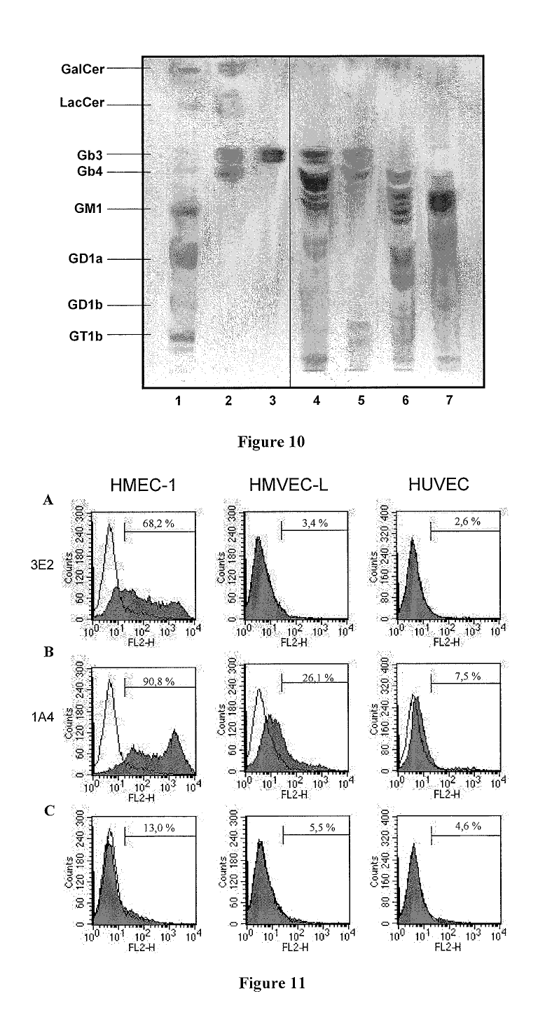

The affinity of antibody 3E2 was evaluated at 30 nM. The number of Gb3 sites is about 2 10⁶ sites for both cell types, but there are slightly more sites on Raji cells than on HMEC-1 cells. Previously, we showed by flow cytometry (FIG. 9, Example 3) that the distribution of Gb3 was homogeneous for Raji cells, for which 87.9% of cells were positive, and heterogeneous for HMEC-1 cells, for which 68.2% of cells were positive. Indeed, within the population of HMEC-1 cells, some cells weakly express Gb3 while others express it more strongly, unlike Raji cells

Study of the Specificity of Antibody 3E2

By an Immunoenzymatic Assay (ELISA) on Desiccated Cells.

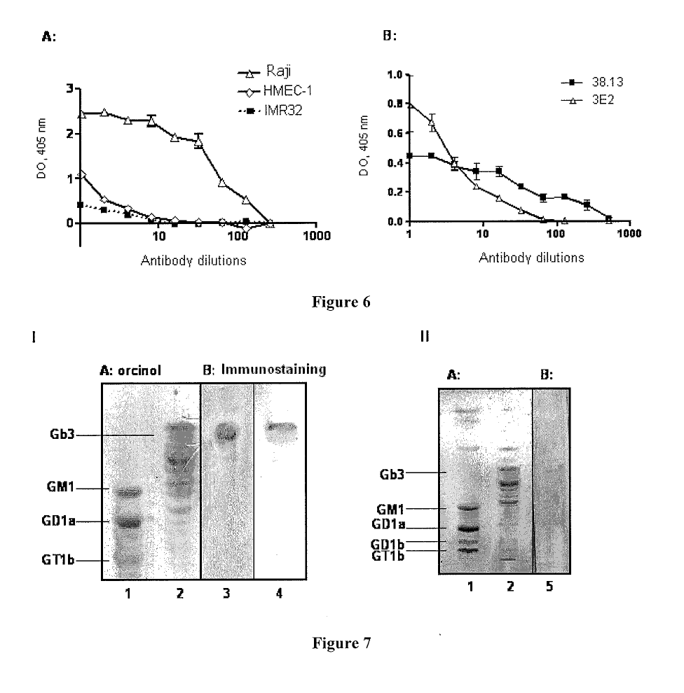

First, the specificity of mAb 3E2 was assessed by ELISA on desiccated Raji, HMEC-1 and IMR32 human neuroblastoma cells that do not express Gb3, using anti-mouse μ chain detection antibodies (FIG. 6).

Antibody 3E2 does not bind IMR32 cells. It binds Gb3-positive HMEC-1 and Raji cells, but their ELISA binding profile is different (A). In fact, antibodies bind more weakly on HMEC-1 cells. This difference could be explained by the mean number of Gb3 sites, which is slightly larger for Raji cells. Moreover, in the HMEC-1 cell population, flow cytometry shows that staining with antibody 3E2 is heterogeneous and that some cells express Gb3 more weakly (FIG. 5, Example 3). It is also conceivable that the Gb3 of HMEC-1 cells is less well recognized when it is in the desiccated form, since it is known that the conformation of a glycolipid can influence its recognition by an antibody.

Despite their low immunogenicity, anti-glycolipid monoclonal antibodies have been obtained by other laboratories. Therefore, we compared the binding of antibody 3E2 with that of rat antibody 38.13. We did not include data on the isotype control antibody and another mouse anti-Gb3 monoclonal antibody 1A4 because they bind non-specifically in the bottom of microtiter plate wells, even after the well saturation step (1 h incubation at room temperature with 1% PBS-BSA) (data not shown). Antibody 38.13 recognizes HMEC-1 cell Gb3. The binding of antibodies 38.13 and 3E2 is not at all identical, since these antibodies are revealed by two detection systems. Binding of antibody 3E2 is revealed by successive incubation of an anti-mouse μ biotinylated secondary antibody followed by incubation of a streptavidin-peroxidase complex, while that of antibody 38.13 is revealed by incubation of an anti-rat μ secondary antibody directly coupled to peroxidase.

Immunostaining of Glycolipids Separated by HPTLC (iTLC).

We compared the chromatographic profile of antibody 38.13 binding with that of antibody 3E2 (FIG. 7).

Antibody 38.13 recognizes HMEC-1 cell Gb3 in the form of a doublet (lane 3), while antibody 3E2 only recognizes the upper band (lane 4). During antibody screening, i.e., before hybridoma cloning, it is found that when immunostaining is done with the antibody 3E2 supernatant, it also recognizes Gb3 in the form of a doublet (lane 5). The assumption is that cloning allows selecting, in a mixture of several hybridomas, a monoclonal antibody only binding the upper band of the Gb3 doublet.

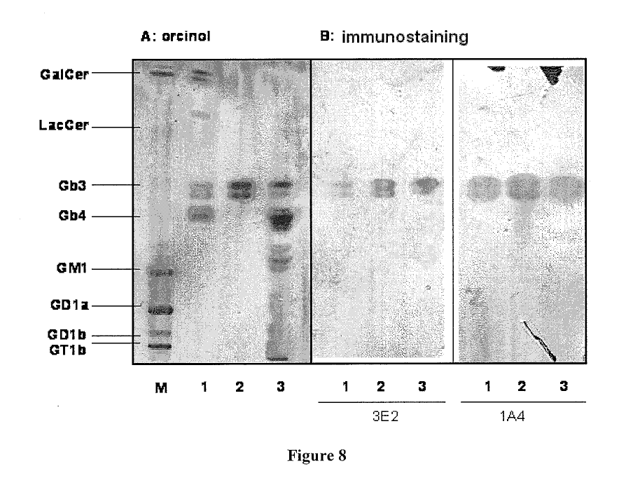

We then compared the two mouse anti-Gb3 monoclonal antibodies: antibody 3E2 generated in the laboratory and antibody 1A4 (FIG. 8).

Mouse 3E2 and 1A4 monoclonal antibodies have different chromatographic binding profiles: antibody 3E2 recognizes the upper band of HMEC-1 cell Gb3, while antibody 1A4 recognizes Gb3 in the form of a doublet (B). In contrast, antibodies 3E2 and 1A4 both recognize purified pig Gb3 (Matreya) in the form of a doublet. Furthermore, it is found that antibody 3E2 binding is weaker than that of antibody 1A4, probably because antibody 1A4 has a better affinity for Gb3 than antibody 3E2.

Study of Cell Staining by Immunofluorescence and Flow Cytometry.

In order to compare the binding of mouse anti-Gb3 antibodies, we stained HMEC-1, Raji and NXS2 cells by flow cytometry using 3E2, 1A4 and isotype control (IgM, κ) antibodies and revealed their binding by anti-mouse μ detection antibodies (FIG. 9 and Table 10).

TABLE 10
Mean fluorescence intensity after flow cytometry analysis.

| Mean fluorescence intensity | HMEC-1 | Raji | NXS2 |
|---|---|---|---|
| mAb 3E2 | 568.3 | 423.8 | 156.5 |
| mAb 1A4 | 984.2 | 498.1 | 117.9 |
| Isotype control | 64.52 | 82.5 | 101.57 |

For HMEC-1 cells, the antibody 3E2 binding profile is different from that of antibody 1A4: 68.2% of cells are positive with antibody 3E2 (A) and 90.8% of cells are positive with antibody 1A4 (B). The mean fluorescence intensity value is 568.3 for antibody 3E2 and 948.2 for antibody 1A4 (Table 10). There are therefore more positive cells when the cells are stained with antibody 1A4, but for both antibodies, the marking of HMEC-1 cells is heterogeneous. In fact, there are two cell populations observed: for antibody 3E2, the less stained population predominates and for antibody 1A4, the more strongly stained population predominates. Raji cell staining is homogeneous and the percentage of positive cells is comparable between the two antibodies (87.9% positive cells and 423.8 for the mean fluorescence intensity value for antibody 3E2, and 93.6% positive cells and 498.1 for the mean fluorescence intensity value for antibody 1A4). Thus, Raji cells strongly express Gb3 homogeneously within the cell population and both antibodies seem to recognize the glycolipid with comparable affinities.

Next, we sought to compare, by HPTLC analysis, the Gb3 content of HMEC-1, Raji and NXS2 cells (FIG. 10).

In HMEC-1 cells, the upper Gb3 band predominates. Antibody 3E2 preferentially recognizes this form compared to antibodies 38.13 and 1A4 which recognize Gb3 in the form of a doublet (FIG. 10). In flow cytometry, after staining HMEC-1 cells with antibody 3E2 (FIG. 10, A), a predominant less stained population is observed. It could also be suggested that in HMEC-1 cells, the molecular species corresponding to the upper Gb3 band is found in a high proportion of cells that express it more weakly than in a minority less stained population. In flow cytometry, after staining HMEC-1 cells with antibody 1A4 (FIG. 10, B), a predominant more stained population is observed. There is then a small proportion of HMEC-1 cells that strongly express the lower form of Gb3 or both forms equally.

When the glycolipid expression profiles of HMEC-1 cells are compared with Raji cells (FIG. 10, lanes 4 and 5), it is found that Raji cells do not have charged glycolipids like gangliosides, they are mostly neutral glycolipids (lane 5). The upper band of Gb3 is strongly predominant. Due to the small content of the species corresponding to the lower band, antibody 1A4 binding is limited to the upper form, which would explain why antibodies 3E2 and 1A4 recognize Raji cell Gb3 equally in flow cytometry.

Mouse neuroblastoma NXS2 cells and T84 cells from a human colon adenocarcinoma, used in the co-culture system with primary HMVEC-L cells do not express Gb3. This result is important because it has already been suggested that glycolipids from the tumor cell microenvironment may be incorporated into endothelial cells after spontaneous release of tumor glycolipids, rendering them more sensitive to VEGF. Since T84 cells do not express Gb3, the antibodies generated by Balb/c mice after immunization clearly recognize an antigen expressed at the surface of HMVEC-L cells and not a glycolipid antigen originating from T84 cells and incorporated into HMVEC-L cells.

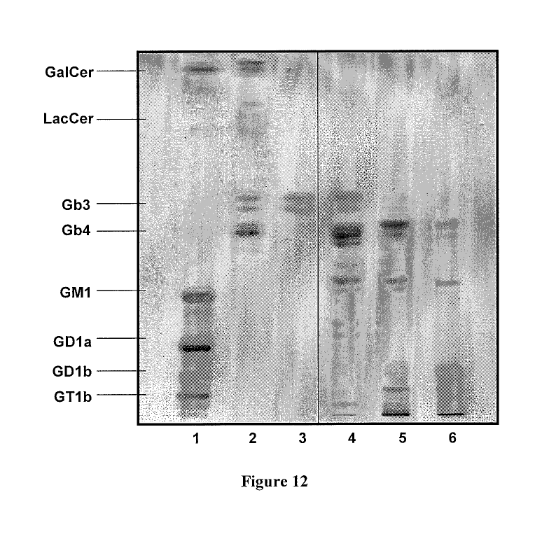

Therefore, we analyzed the Gb3 content of HMVEC-L cells by comparing them by flow cytometry (FIG. 11) and HPTLC (FIG. 12) to the expression profiles of HMEC-1 and HUVEC human macrovascular cells for which the presence of Gb3 has already been established in the literature (Obrig et al. 1993; Müthing et al. 1999; Kanda et al. 2004).

TABLE 11
Mean fluorescence intensity after flow cytometry analysis.

| Mean fluorescence intensity | HMEC-1 | HMVEC-L | HUVEC |
|---|---|---|---|
| mAb 3E2 | 568.3 | 67.6 | 33.4 |
| mAb 1A4 | 984.2 | 180.7 | 199.7 |
| Isotype control | 64.52 | 83.8 | 60.3 |

Whether by flow cytometry (FIG. 11) or HPTLC analysis (FIG. 12), it is found that primary HMVEC-L and HUVEC endothelial cells express less Gb3 than transformed HMEC-1 endothelial cells

By flow cytometry, antibody 3E2 does not recognize non-co-cultured HMVEC-L cells (3.4% of positive cells). This absence of binding is due to an overall reduction of the Gb3 doublet observed by HPTLC, since even though the overall Gb3 content is low, the upper band remains predominant in HMVEC-L cells. Since antibody 1A4 binds HMVEC-L cell Gb3 (26.1% of positive cells) and since the upper band predominates, the difference in binding is due to a difference of affinity between the two antibodies. The affinity of antibody 1A4 for Gb3 seems superior to that of antibody 3E2, since the low Gb3 content that can be visualized in HPTLC can only be detected by flow cytometry with antibody 1A4. In HUVEC cells, almost no Gb3 is detected by HPTLC. In flow cytometry, only 7.5% of cells are positive with antibody 1A4 instead of 2.5% of positive cells with antibody 3E2.

HPTLC analysis shows that the overall neutral glycolipid content is limited in HUVEC cells (lane 6). For HMVEC-L cells, it is the proportion of Gb3 within the total fraction of neutral glycolipids that is limited (lane 5) since these cells strongly express a glycolipid present in the form of a doublet probably corresponding to glycolipid Gb4. For HMVEC-L and HUVEC cells, it is the upper band of globoside Gb4 that is predominant, while for HMEC-1 cells, it is the lower band of Gb4 that is predominant. Primary cells have higher contents of complex gangliosides migrating below GD₁ₐ. Moreover, for the three cell types, it is the ganglioside migrating above GM1 that would be most abundant in the total ganglioside fraction. Since the preponderance of GM₃ in the total ganglioside fraction has already been demonstrated in human endothelial cells Obrig et al. 1993; Müthing et al. 1999; Kanda et al. 2004), it is probable that the preponderant ganglioside in HMEC-1, HMVEC-L and HUVEC cells is GM₃.

Immunofluorescence on Cells in Culture.

We analyzed the cellular localization of Gb3 by immunofluorescence. The results show that antibody 3E2 is suited to the study of cells by immunofluorescence. The advantage of this technique is that one can observe the antigenic distribution of living cells directly in their culture substrate, without their being trypsinized before staining, as is the case in flow cytometry. Depending on the results obtained with the isotype control, the signal obtained may not originate from non-specific interactions. It is observed that the distribution of Gb3 is localized at the cell membrane and that its membrane distribution is fairly homogenous within the cell (data not shown).

Immunohistochemistry on Frozen Tumor Sections.

We analyzed, by immunohistochemistry, the distribution of Gb3 in frozen Raji tumor sections expressing Gb3 and IMR32 tumors not expressing Gb3, which we verified beforehand by ELISA with antibody 3E2 and by flow cytometry. These tumors were implanted with Matrigel and harvested at the end of 3 to 4 weeks.

The results (not shown) indicate that antibody 3E2 is an immunohistochemical tool that appears to meet expectations. It recognizes the presence of Gb3, which is located at the membrane of Gb3-positive tumors (Raji) and does not show non-specific binding on tumors not expressing it (IMR32). The isotype control antibody is also appropriate for immunohistochemical studies since it does not present a specificity.

Nucleotide Sequences of VII and VL Variable Regions of Antibody 3E2.

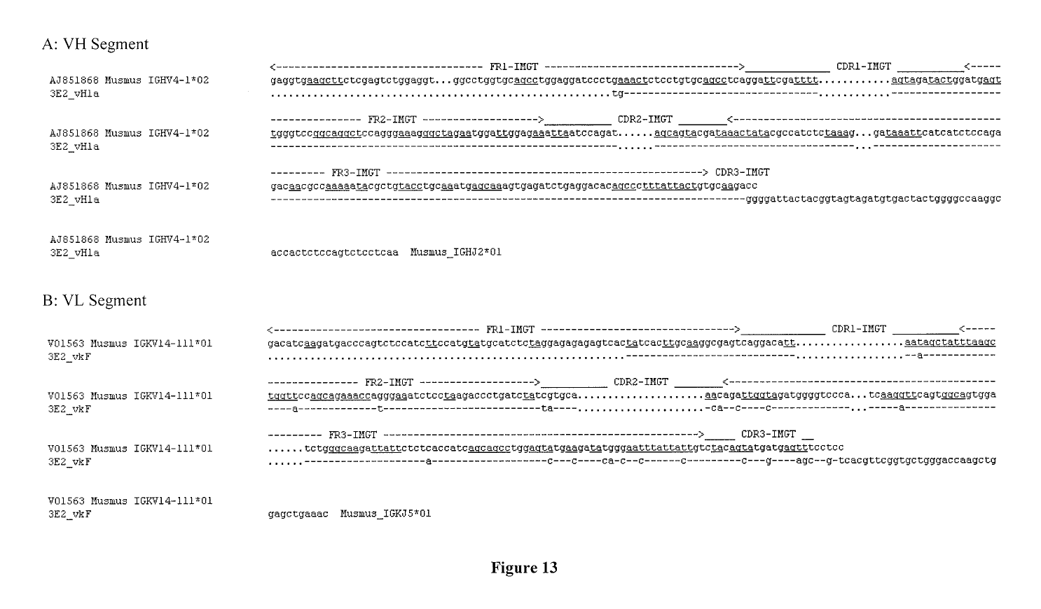

In order to finalize the characterization of antibody 3E2, we analyzed, after RT-PCR, the nucleotide sequences of the V_{H} and V_{L} variable regions in order to align them with data from the IMGT (International Immunogenetics Information System®) bank. The primers used for sequencing the heavy and light chains are listed in Table 12. The heavy and light chain nucleotide sequences are shown in FIG. 13 and the genes used for these variable regions are listed in Table 13.

TABLE 12
Back primers used for amplification of the variable regions
of the V_{H} and V_{L} gene segments of antibody 3E2.

| | |
|---|---|
| Heavy chain | Light chain |

| | PCR | Back | PCR | Back |
|---|---|---|---|---|
| Hybridoma | conditions | primer | conditions | primer |
| 3E2 | a | VH1 A | e | VκF |

TABLE 13
Genes used for the light chain variable regions.

| mAb | V Gene | V Region | J Gene | J Region | D Gene | D Region |
|---|---|---|---|---|---|---|
| 3E2_VH1a | IGHV4-1*02 | 99.15% (233/235 nt) | IGHJ2*02 | 65.96% | IGHD1-1*01 | 78.57% |
| | | | | (31/47 nt) | | (11/14 nt) |
| 3E2_VκF | IGKV14-111*01 | 90.91% (200/220 nt) | IGKJ5*01 | 100% | / | / |
| | | | | (38/38 nt) | | |

Monoclonal antibody 3E2 has 99% homology with the IGHV4 gene of germ-line configuration (genomic DNA). Two nucleotides that are mutated are noted in the FR1 region. The antibody has 65.96% homology with the IGHJ2 gene and 78.57% homology with the IGHD1 gene. The V_{K} of the antibody has a 90.91% homology over 220 nucleotides with the IGKV14 gene and has 100% homology with the IGKJ5 gene. Due to the differences observed with germ-line configuration genes, it is demonstrated that antibodies undergo a maturation process marked by several somatic mutations.

4.2 Conclusion

We obtained several hybridoma lines by somatic hybridization, including antibody 3E2 (IgM,κ) directed against neutral glycolipid Gb3. We continued its characterization using Raji human lymphoma cells for which Gb3 is a marker, human IMR32 and mouse NXS2 neuroblastoma cells that do not express Gb3 and primary endothelial cells, lung microvascular cells HMVEC-L and umbilical cord macrovascular cells HUVEC.

We determined by Scatchard representation that the antibody showed good affinity of around 30 nM. In HMEC-1 cells, we showed the membrane localization of Gb3 by immunocytochemistry, and then showed its capacity to specifically bind Gb3 by immunohistochemistry on frozen Raji tumor sections. We analyzed its specificity with ELISA on desiccated cells and by flow cytometry and immunostaining on glycolipids separated by HPTLC. Antibody 3E2 is a new tool for Gb3 analysis, in addition to the antibodies that have already been generated, such as the rat IgM 38.13 and mouse IgM 1A4 antibodies obtained by Prof S. Hakomori's team. It is these antibodies that permitted establishing Gb3 as a Burkitt lymphoma marker, showing that Gb3 was involved in apoptosis phenomena in lymphoma Gb3-positive B cells and that screening for Gb3 by antibodies (Taga et al. 1997; Tetaud et al. 2003) or by verotoxin recombinant subunit B (Mangeney et al. 1993) can also induce an apoptosis response. A third antibody, BGR-23 obtained more recently (Kotani et al. 1994) was used to study the tissue distribution of Gb3 in Fabry disease (Askari et al. 2007). These antibodies are specific for the terminal motif Gal α1→4 Gal β and cannot recognize (1) the motif Gal α1→3 Galβ present at the terminal end of the oligosaccharide chain of isoglobotriaosylceramide (iGb3) and (2) the motif Gal α1→4 Gal β present inside the oligosaccharide chain of globoside Gb₄ (GalNac β1→3 Gal α1→4 Gal β1→4 Glc β1→Cer).

During the analysis of specificity, we demonstrated the following characteristics. Antibody 3E2 recognized, on HPTLC, the upper band of the Gb3 doublet of HMEC-1 cells and pig Gb3 in the form of a doublet, unlike rat 38.13 and mouse 1A4 anti-Gb3 antibody, which recognize both molecular species of Gb3 in HMEC-1 cells and pig Gb3. The molecular species corresponding to this upper band is expressed heterogeneously in HMEC-1 cells. There are two cell populations: a predominant population that more weakly expresses this Gb3 and a minority population that strongly expresses this Gb3. Antibodies 3E2 and 1A4 equally recognize the Gb3 of Raji cells which express the strongly predominant upper form at their surface, and do so homogenously, since all these cells are strongly stained. Moreover, in an in-vitro model, it was very clearly shown that the incorporation of GD₁ₐ in HUVEC cells rendered these cells more sensitive to small contents of VEGF. They suggest that, in the tumor microenvironment, free tumor cell glycolipids and glycolipids incorporated in endothelial cells would promote tumor progression through a process of shedding. Since T84 cells do not express Gb3, the antibodies generated after immunization were directed against the Gb3 expressed by HMVEC-L endothelial cells from co-culture.

Regarding primary endothelial cells, it is found that the presence of Gb3 is reduced overall, independently of the form of ceramide. This expression difference with HMEC-1 cells may be due to the tissue origin of the cells. Human renal microvascular endothelial cells (HRMEC) express 48 times more Gb3 than human umbilical cord vascular endothelial cells (HUVEC) (3.2 nmoles/10⁶ HRMEC cells and 0.067 nmoles/10⁶ HUVEC cells) (Obrig et al. 1993). Human brain microvascular endothelial cells (HBMEC) express 2 times more Gb3 than human umbilical cord vascular endothelial cells (HUVEC) (612±185 and 269±62 ng/mg of protein) (Kanda et al. 2004).

Tumor transformation may also influence the content of a glycolipid, as shown by the abnormal overexpression of disialoganglioside acid GD2 in neuroblastomas, most melanomas and some other tumors or overexpression of Gb3 in several tumor cell lines such as Burkitt lymphoma (Wiels et al 1981), of cells from breast and ovarian cancers, astrocytic tumor cells (Gariepy, 2001. LaCasse et al. 1999), cells from epithelial carcinoma of the upper digestive tract (Marques Filho et al. 2006.) or in human tumor tissues, such as breast cancer (Johansson et al. 2009), colorectal tumors or metastases (Falguieres et al. 2008). The fact that primary endothelial cells express less Gb3 than transformed cells is an interesting result because we want to target proangiogenic endothelial cells while sparing the endothelial cells of healthy tissue. Moreover, we analyzed the glycolipid content of HMVEC-L cells cultured alone in the absence of co-culture with other cell types and it was shown that the presence of tumor cells in co-culture induced a phenotype and genotype change in primary endothelial cells (Khodarev et al. 2003).

The biochemical changes observed during malignant transformation of cells also affect ceramide biosynthesis. These changes manifest by variations in the length of the aliphatic chain, the number of substitutions or the degree of unsaturation. While in healthy tissues, fatty acid chains are shorter (C14:0 to C18:0), the gangliosides of tumor cells have longer fatty acid chains (C22:0, C22:1 and C24:1) (Hakomori and Kannagi, 1983). An aberrant α-hydroxylation of tumor ganglioside fatty acids may also be observed.

It is usual to find glycosphingolipids present in the form of a doublet on chromatograms stained with orcinol. These doublets reflect the presence of various substitutions, the length of the fatty acid chain and differences in hydroxylation and saturation in the glycolipid ceramide, which can then migrate differentially by HPTLC. Pig Gb3 obtained from Matreya is made up of a mix of two types of ceramide chains (C:16 and C:20) which are composed of 70% saturated fatty acids and 30% unsaturated fatty acids. These two forms are recognized by antibody 3E2 and it is very likely that the Gb3 doublet found in HMEC-1 cells is itself made up of two types of fatty acid chains and that the lower form that is not recognized by the antibody has yet another fatty acid chain. It has already been shown by HPTLC of HUVEC cells that Gb3 was present in the form of a doublet that corresponded to two types of ceramide chains (C24:0) and (C16:0) determined by mass spectrometry (Müthing et al. 1998). In addition to the tissue origin of vascular cells, it appears that the glycolipid profile varies from one species to another. Only traces of Gb3 can be detected in primary bovine aortic endothelial cells BAEC, in which different types of fatty acid chains ranging from C24:0 or C24:1 to C:16 could be detected.

The presence of these hydroxyl groups as well as variations in the length of the ceramide change the conformation of the antigen and thus its accessibility to the antibody. The affinity of anti-glycolipid antibodies would increase as a function of the ceramide fatty acid chain length, which could be the case for the upper form of Gb3 recognized by antibody 3E2 in HMEC-1 cells, while the antibody does not have an affinity for the lower form. An anti-GD3 antibody (mAb 4.2) obtained after immunization of mice with human melanoma cells containing a large amount of C:24 GD₃ only slightly binds cells comprising low contents of C:24 GD₃. Moreover, it has been shown that monoclonal antibodies directed against glycolipids were able to recognize high-density antigens and that these same antibodies would not be able to react with the same antigens of low density.

The data regarding the differences in antibody binding need to be correlated with studies that show that the density, and not just the presence of Gb3, is necessary for binding Shiga-like toxin. Other studies show that Shiga toxin subunit B (StxB) is induced by binding a Gb3 cluster at the membrane, which is responsible for invagination of the membrane and allows internalizing the subunit and bringing it to the intracellular compartment. This is made possible because StxB is homopentameric, which is also the case for verotoxin. The vast majority of antibodies that we selected belong to isotype IgM. Due to its pentavalence, immunoglobulin M mimics the pentameric structure of these toxins and gathers together the glycolipids naturally present in the form of lipid rafts, according to synthetic plasma membrane models. These data explain why an antibody of isotype IgG1 like antibody 12E10.1c selected by the second screening strategy showed a poor affinity for Gb3, as shown by the weak binding of this antibody on HMEC-1 cell Gb3.

It has already been demonstrated that the molecular structure of Gb3, i.e., its fatty acid composition, determines the location of lipids in the lipid bilayers, and considerably contributes to the formation of Gb3 clusters. In the case of synthetic bilayers composed of phosphatidylcholine and Gb3, this observation is corroborated by the fact that the affinity of STxB increases if the length of the phosphatidylcholine fatty acid chain decreases, which generates a greater exposure of Gb3 at the surface of this bilayer. Thus, in the same way as antibodies, molecular modeling studies have shown that fatty acid composition influences the conformation of Gb3 at the membrane interface and that had a direct incidence on STxB affinity. Verotoxin binding also depends on the length of Gb3 ceramide fatty acid chains. Glycolipids with medium or long fatty acid chains (C16, and C24) are recognized preferentially while short chains (C12 and C14) show minimal binding. Fatty acid chains C20:0 and C22:1 have a greater binding capacity and the presence of unsaturated fatty acids also significantly increases verotoxin binding. Fatty acid hydroxylation also increases binding. Verotoxin binding at the terminal motif Gal (α1-4) Gal of glycolipids depends on the whole structure of the molecule and its molecular environment in the plasma membrane of target cells. This result proves that glycolipids are involved in cellular tropism closely related to their structure.

Antibody 3E2 thus targets an original glycolipid present in endothelial cells. It would be important to determine the structure of the two molecular species of Gb3 present in HMEC-1 and HMVEC-L in order to show the structural variations that are responsible for the differential specificity of antibodies 3E2 and 1A4. Previously, we showed that Gb3 expression was heterogeneous in HMEC-1 cells and that it appeared to be modulated as a function of the proliferation state of these cells. It is necessary to see whether these results can be corroborated with a biological activity to determine the therapeutic potential of the antibody.

### Example 5

### In Vitro Biological Properties of Anti-Gb3 mAb 3E2

We also want to see if this antibody can have biological properties and if it can be a new therapeutic tool.

5.1 Results

Study of Gb3 Expression Polymorphism by Flow Cytometry with Antibody 3E2.

We previously demonstrated, by HPTLC (see Example 2), that the content of glycolipid Gb3 could be modulated when cells were proliferating, either incubated in complete medium or incubated in depleted medium supplemented with a pro-proliferative factor, sphingosine-1-phosphate (S1P). Since antibody 3E2 is a new analysis tool, we again analyzed the expression of Gb3 in HMEC-1 cells by two different methods, which differ from HPTLC analysis, enabling the total glycolipids in cells to be analyzed independently of their location in the cell. By flow cytometry, the homogeneity of Gb3 expression at the cell surface can be analyzed, and by the Scatchard technique, the number of Gb3 cites at the cell surface can be determined.

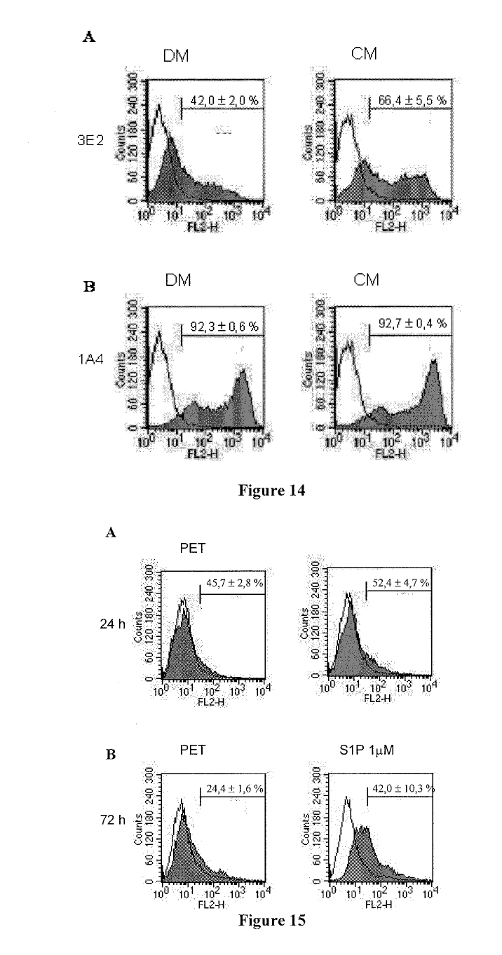

First, we analyzed, by flow cytometry, the expression of Gb3 after 24 h of incubation in depleted or complete culture medium (FIG. 14).

We confirmed by cytometry that the Gb3 content decreases when HMEC-1 cells are incubated for 24 h in depleted medium. In fact, in complete medium, there are two cell populations seen after staining with antibody 3E2. When cells are incubated in depleted medium, there are fewer cells stained (66.4±5.5% in complete medium and 42.0±2.0% in depleted medium (n=3)) until there is virtually only a single cell population that weakly expresses Gb3. The mean fluorescence intensity is decreased by more than 50% (Table 14).

TABLE 14
Percentage of positive cells and mean fluorescence
intensity after flow cytometry analysis.

| | HMEC-1 cells | | | |
|---|---|---|---|---|
| | DM | | CM | |
| | | Mean | | Mean |
| | Number of | fluorescence | Number of | fluorescence |
| | positive cells | intensity | positive cells | intensity |
| mAb 3E2 | 42.0 ± 2.0% | 300.7 ± 3.8 | 66.4 ± 5.5% | 639.6 ± 2.6 |
| mAb 1A4 | 92.3 ± 0.6% | 1046.0 ± 14.0 | 92.7 ± 0.4% | 1356.9 ± 39.7 |

The reduction in Gb3 content is less extensive when the cells are stained with antibody 1A4. Although this result is preliminary, it seems to suggest that the upper band of the Gb3 doublet is especially overexpressed in complete medium, i.e., when endothelial cells are proliferating. This would show that antibody 3E2, which specifically recognizes this upper band, and any other antibody with this same specificity, would preferentially recognize proliferating endothelial cells, unlike antibodies recognizing both bands of the doublet.

We also analyzed, by immunocytochemistry with antibody 3E2, 24 h after their seeding, the distribution of membrane Gb3 in two interacting HMEC-1 cells cultured in their complete medium. It is found that the distribution is not homogenous, as can be seen when the cell is isolated (data not shown). There is membrane polarization of Gb3 in the contact zone between cells and there are also more isolated cells, more weakly stained (indicated by the white arrows). Thus, in addition to the heterogeneity of HMEC-1 cell staining within the cell population, there is also heterogeneity of the Gb3 distribution in the membrane of interacting cells. The presence of isolated cells indicated by the white arrows, which are more weakly stained by antibody 3E2, confirms the presence of the cell populations observed by flow cytometry, which express Gb3 in a different way at their surface.

We then analyzed, by flow cytometry with antibody 3E2, the expression of Gb3 in HMEC-1 cells incubated for 24 h and 72 h in depleted medium supplemented with sphingosine-1-phosphate or its diluent, PET (FIG. 15).

TABLE 15
Percentage of positive cells and mean fluorescence
intensity after flow cytometry analysis.

| | HMEC-1 cells | | | |
|---|---|---|---|---|
| | PET | | S1P | |
| | | Mean | | Mean |
| | Number of | fluorescence | Number of | fluorescence |
| | positive cells | intensity | positive cells | intensity |
| 24 h | 45.7 ± 2.8% | 91.1 ± 46.3 | 52.4 ± 4.7% | 104.4 ± 37.6 |
| 72 h | 24.4 ± 1.6% | 184.0 ± 2.5 | 42.0 ± 10.3% | 212.3 ± 24.1 |

When cells are incubated in the presence of S1P, the Gb3 content at the surface of HMEC-1 cells slightly increases. In flow cytometry, this content is only detectable at the end of 72 h, while this increase is seen by 24 h by HPTLC (FIG. 1C). The cells are positive at a rate of about 42.0±0.3% after incubation with S1P, while only 24.4±1.6% are positive in the presence of depleted medium supplemented with the diluent, PET. When cells are incubated in complete medium, the increase in Gb3 content is greater (FIG. 14). The fetal calf serum present in complete medium has a mixture of growth factors and this increase may be due to a synergistic action of growth factors.

We then evaluated, by the Scatchard technique, the number of Gb3 sites at the surface of HMEC-1 cells incubated 24 h in depleted medium or in complete medium. The analysis of the saturation curve according to the Scatchard equation allowed us to precisely determine the number of Gb3 sites of HMEC-1 cells recognized by antibody 3E2 (Table 16).

TABLE 16
Number of Gb3 sites evaluated by antibody 3E2 (Kd =
30 nM) for HMEC-1 cells after incubation in depleted
culture medium (DM) and complete medium (CM).
Number of sites/cell

| | |
|---|---|
| DM | 0.2 10⁶ |
| CM | 1.7 10⁶ |

There are 8.5 times more Gb3 sites when HMEC-1 cells are incubated for 24 h with complete culture medium than when the cells are incubated with depleted culture medium.

It clearly appears that the Gb3 content of proliferating cells is modulated: the mean fluorescence intensity of the cells is increased by more than 50% (FIG. 14); a polarization of Gb3 is observed when HMEC-1 cells in culture are in contact, while isolated HMEC-1 cells are more weakly stained (data not shown) and there are 8.5 times more Gb3 sites at the surface of proliferating HMEC-1 cells (Table 15). These results confirm once again the interest of generating anti-Gb3 antibodies; it remains to determine the biological properties of the antibodies.

In-Vitro Biological Properties of Mouse Monoclonal Antibody 3E2.

Cytostatic Properties of Antibody 3E2

Study on Cellular Viability (MTT) of Cells Treated with Antibody 3E2.

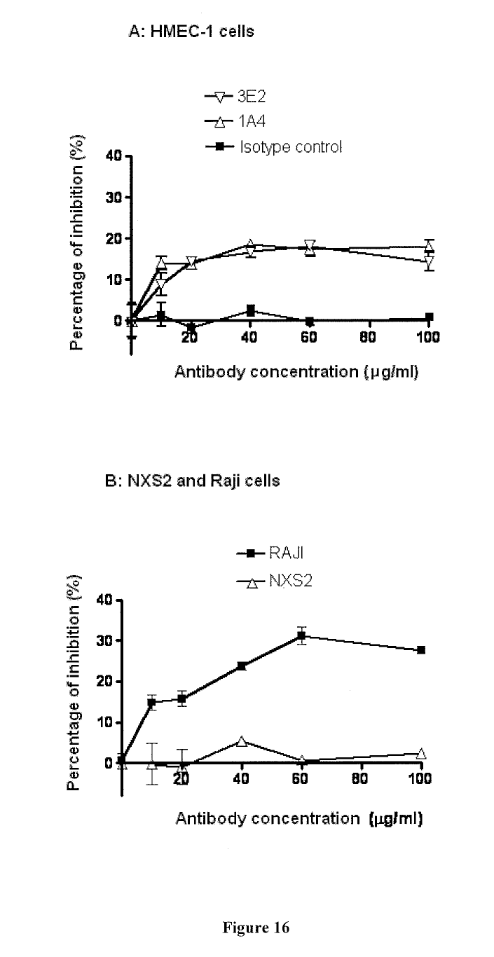

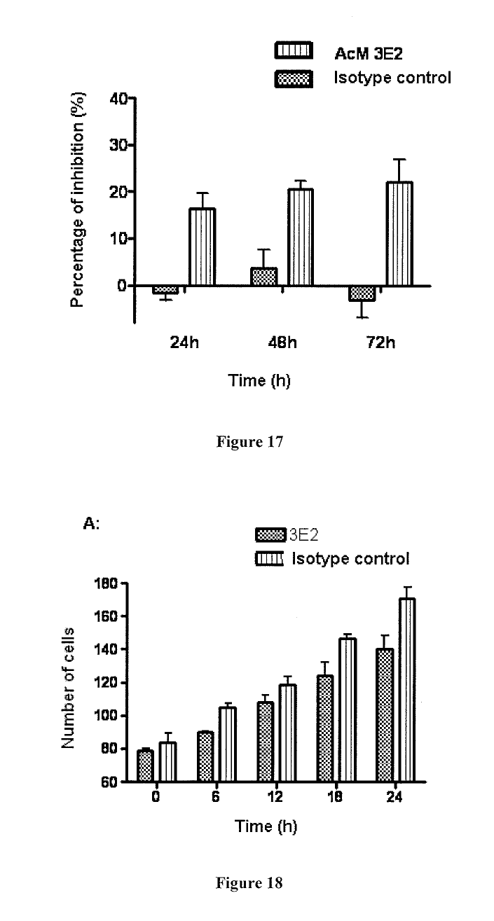

One the one hand, we measured the inhibition of cellular viability of HMEC-1 and Raji cells after 24 h of incubation with antibodies 3E2 and 1A4 at different concentrations (FIG. 16) and, on the other hand, the inhibition kinetics of cellular viability of HMEC-1 cells after 24 h, 48 h and 72 h of incubation with 20 μg/ml of antibody 3E2 (FIG. 17).

At 24 h (FIG. 16, A), the inhibition of cellular viability induced by antibodies 3E2 and 1A4 is comparable and leads to a saturation plateau at 20 to 40 μg/ml of antibodies, for which equal values are reached at 14.6±1.6% and 16.3±3.1% inhibition. No inhibition of cellular viability is observed with the isotype control antibody on HMEC-1 cells. This inhibition is greater for Raji cells (B) which have more Gb3 sites (2.0 10⁶ sites versus 1.7 10⁶ sites for HMEC-1 cells) and which express Gb3 homogeneously. Within the population of HMEC-1 cells, certain cells express Gb3 more weakly, which could explain why the inhibition of viability is weaker. No inhibition is observed for NXS2 cells, which do not express Gb3. The inhibition of cellular viability is therefore dependent on Gb3.

We observed the inhibition of cellular viability for 72 h at a single dose of 20 μg/ml of antibody 3E2 (FIG. 17). The maximum value of cellular viability inhibition is almost reached by 24 h of incubation. At 72 h, it is found that cellular viability inhibition is slightly greater (22.2±9.1%). For antibody 1A4, the inhibition kinetics are comparable to those of antibody 3E2 (data not shown). No inhibition of cellular viability is seen with antibody 3E2 on NXS2 cells that do not express Gb3, at 48 h and at 72 h (data not shown).

Video-Kinetic Study of HMEC-1 Cells Treated with Antibody 3E2.

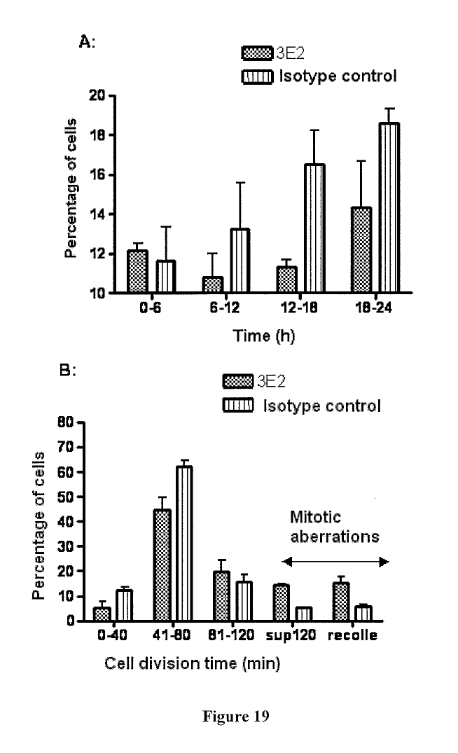

We sought to determine the nature of the cellular viability inhibition observed by performing an accelerated video-kinetic study of HMEC-1 cells for 24 h in the presence of 20 μg/ml of antibody 3E2 or 20 μg/ml of isotype control antibody. For each of the conditions, we compared the number of HMEC-1 cells per time interval (FIG. 18), the amount of cells in division per time interval and the cell division time (FIG. 19).

FIG. 18 shows the number of HMEC-1 cells as a function of incubation time with 20 μg/ml of antibody 3E2 or of the isotype control antibody. It is found that there are fewer HMEC-1 cells in the presence of antibody 3E2 at 24 h of incubation. This reduction is significant by 6 h of incubation (A). At the end of 24 h, a reduction in the number of cells is observed that can be evaluated at around 18%, which is close to the 14.6% cellular viability inhibition that is observed at 24 h by MTT (FIG. 16).

FIG. 19 shows the proportion of cells that enter into cell division per time interval (A) and their cumulative division time during these 24 h of incubation (B). Indeed, cells entering into division can be detected and counted: the cells lose their adhesion, detach from the substrate, become more refractive and adopt a circular morphology, while they have cytoplasmic expansions when they are adherent. Thus the cells in division and their division time until the daughter cells adhere to the substrate again can be identified.

It is found that the number of HMEC-1 cells is division is smaller when the cells are incubated in the presence of antibody 3E2 (A). This reduction is significant by 12 h. When their cellular division time over 24 h of incubation is analyzed, it is found that this time increases overall (B). In the presence of antibody 3E2, there are fewer cells which take between 0 and 40 min or between 41 and 80 min to divide, while there are more cells that take more than 80 min to divide. The presence of cells that take more than 120 min to divide is also noted (“more than 120” B), cells that do not finish their division before the end of 24 h of incubation and cells that do not enter into cell division (“stuck”, B). We have classified these cells in the category of mitotic aberrations (FIG. 19, B).

Analysis of the images recorded corresponding to the division times of cells incubated in the presence of the isotype control and antibody 3E2 shows that the cell indicated by the arrow takes around 40 min to divide in the presence of the isotype control and another cell incubated with antibody 3E2 takes 130 min (data not shown).

The analysis of recorded images of cells classified in the category of mitotic aberrations shows the presence of cells that do not finish their division in 24 h of incubation and the fate of such a cell observed at 27 h [sic; 72 h?], which finally bursts in 120 min.

Among the mitotic aberrations that we observed, the presence of significantly more cells that try to enter cell division without being able to do so is also noted. These cells detach, become refractive and then adhere again, successively.

Antiangiogenic Properties of Antibody 3E2.

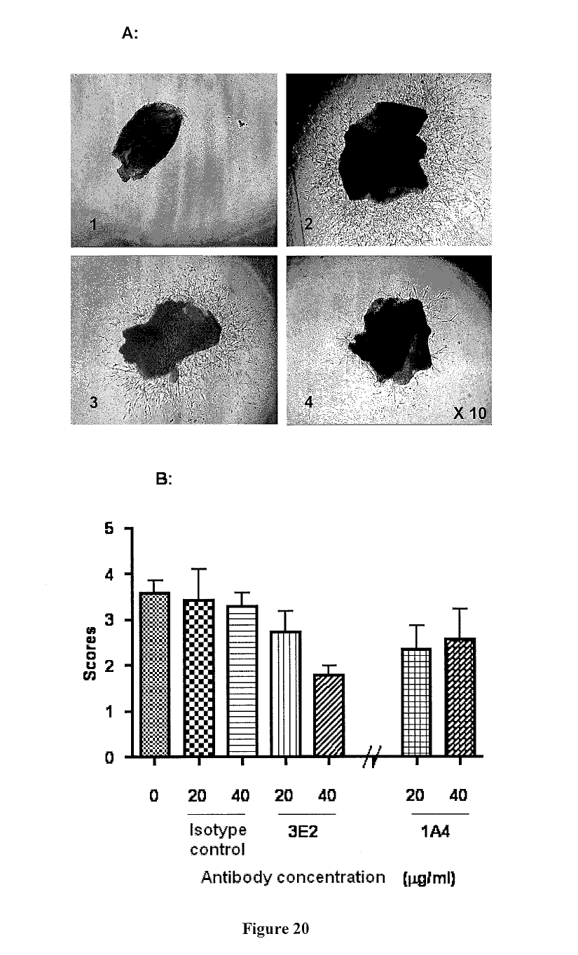

We evaluated the antiangiogenic properties of antibody 3E2 by the aortic ring test which is the standard ex vivo test to measure the pro- or antiangiogenic activity of molecules (FIG. 20).

At the end of five days of incubation of the rings in complete medium without antibody (A, photo 2), the formation of a large number of vascular buds is observed, characterized by their length and density. When the rings are incubated with antibody 3E2 (FIG. 20, B), an inhibition of bud formation is seen from 20 μg/ml, which is significant at 40 μg/ml. This inhibition is dependent on Gb3; antibody 1A4 has a comparable antiangiogenic activity to antibody 3E2, while the isotype control antibody does not significantly reduce bud formation.

Cytotoxic Properties of Antibody 3E2 in the Presence of Human Complement (CDC)

Previously, we evaluated antibody 3E2 as having cytostatic activity: it induces an inhibition of cellular viability at 24 h, a reduction in the number of cell divisions and an extension of division time. It also induces an inhibition of the formation of vascular buds on cross sections of aortic explants in culture. These observations were obtained in the absence of complement. We sought to measure the CDC (complement-dependent cytoxicity) activity of the antibody by flow cytometry according to a method developed in the laboratory by using the ability of propidium iodide to bind cell DNA. If the cell membranes were damaged by CDC activity, their DNA will be accessible and propidium iodide can be seen by flow cytometry.

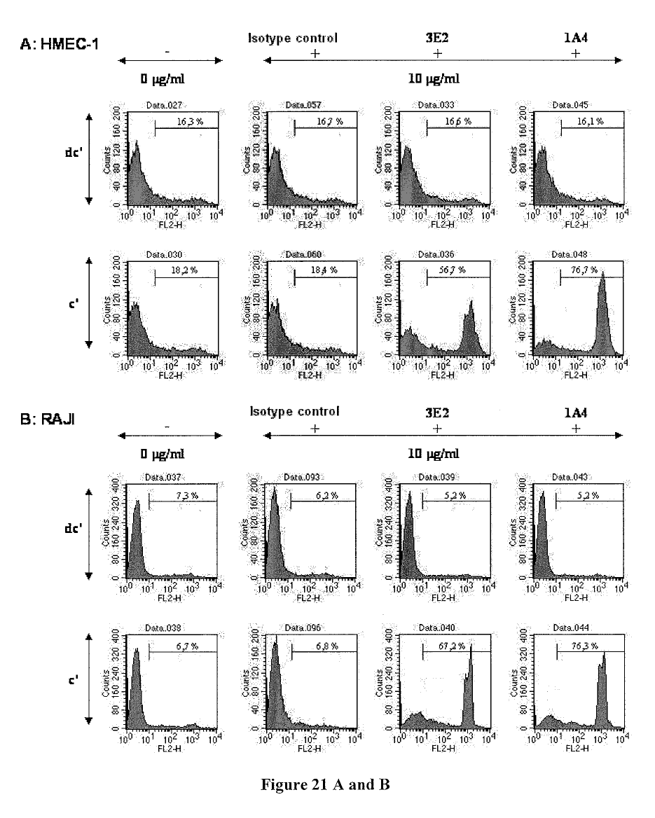

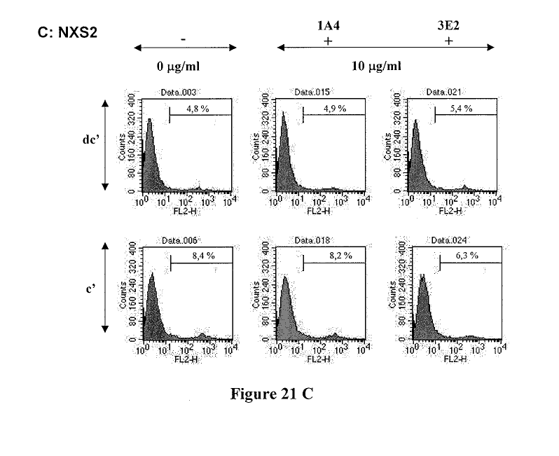

FIG. 21 shows the CDC activity of antibodies 3E2, 1A4 and the isotype control antibody (10 μg/ml of antibody) on HMEC-1, Raji and NXS2 cells, in the presence of decomplemented and non-decomplemented human serum.

Table 17 lists the percentage of cells lysed for antibody concentrations ranging from 0.1 to 10 μg/ml (mean of 3 independent experiments).

TABLE 17
Percentages of cells lysed (n = 3) after binding of antibodies 3E2 and
1A4 on HMEC-1, Raji and NXS2 cells.

| | 3E2 | 1A4 | ISO | — | 3E2 | | | | 1A4 | | | |
|---|---|---|---|---|---|---|---|---|---|---|---|---|
| mAb | + | + | + | − | + | + | + | + | + | + | + | + |
| mAb | 10 | 10 | 10 | — | 0.1 | 0.5 | 5 | 10 | 0.1 | 0.5 | 5 | 10 |
| concentration | | | | | | | | | | | | |
| (μg/ml) | | | | | | | | | | | | |
| Non- | − | − | + | + | + | + | + | + | + | + | + | + |
| decomplemented | | | | | | | | | | | | |
| serum | | | | | | | | | | | | |
| HMEC-1 | 17.5 ± | 18.6 ± | 18.1 ± | 16.5 ± | 16.4 ± | 20.6 ± | 39.8 ± | 51.3 ± | 22.2 ± | 37.2 ± 6.21 | 56.9 ± 6.51 | 65.2 ± 2.31 |
| | 5.4 | 6.5 | 5.4 | 7.2 | 7.2 | 2.3 | 6.11 | 2.9 | 9.6 | | | |
| Non- | − | − | + | + | + | + | + | + | + | + | + | + |
| decomplemented | | | | | | | | | | | | |
| serum | | | | | | | | | | | | |
| Raji | 10.8 ± | 11.1 ± | 11.6 ± | 10.8 ± | 15.7 ± | 36.4 ± | 67.4 ± | 71.8 ± | 25.8 ± | 68 ± 6.6 | 83.6 ± 7.7 | 82.9 ± 7.7 |
| | 7.5 | 8.5 | 9.5 | 1.3 | 2.5 | 8.6 | 6.4 | 5.5 | 9.3 | | | |
| Non- | − | − | + | + | + | + | + | + | + | + | + | + |
| decomplemented | | | | | | | | | | | | |
| serum | | | | | | | | | | | | |
| NXS2 | 10.3 ± | 10.7 ± | ND | 8.3 ± | ND | ND | ND | 5.55 ± | ND | ND | ND | 7.05 ± 1.1 |
| | 7.0 | 6.0 | | 1.0 | | | | 9.0 | | | | |

ND: not determined. ISO: isotype control.
In the absence of non-decomplemented serum, the cells are incubated with decomplemented serum.

At 10 μg/ml, antibody 3E2 shows a CDC activity on HMEC-1 cells (Table 17, 51.3±9.2% of cells lysed) and Raji cells (71.8±5.5% of cells lysed). The activity can be detected at low concentrations of around 0.5 μg/ml of antibody (20.6±3.2 of HMEC-1 cells lysed and 36.4±6.8% of Raji cells lysed). Antibody 1A4 also has significant CDC activity at 10 μg/ml (65.2±13.2% of positive HMEC-1 cells and 82.9±7.7% of positive Raji cells). CDC activity can be detected from 0.1 μg/ml of antibody (37.2±12.6% positive HMEC-1 cells and 25.8±3.9% positive Raji cells).

NXS2 cells, which do not express Gb3, are not lysed in the presence of antibodies 3E2 and 1A4 and HMEC-1 and Raji cells, which express Gb3, are not lysed in the presence of the isotype control: thus the cytotoxic activity observed is clearly dependent on Gb3. In the absence of antibody, NXS2, HMEC-1 and Raji cells are not lysed in the presence of non-decomplemented serum: there is therefore no activation of the alternative complement pathway, which plays an important role in non-specific innate immune defense. HMEC-1 and Raji cells are not lysed in the presence of antibodies 1A4 or 3E2 when they are incubated in the presence of decomplemented serum or in the absence of human serum: no necrotic or apoptotic cell death is observed. The cytolytic activity observed is therefore clearly dependent on the activation of complement, via the binding of antibodies on Gb3.

Simultaneously with the staining with propidium iodide, which reflects the CDC activity of the antibodies, we measured Gb3 expression by flow cytometry, in order to evaluate the CDC activity/cell staining ratios for 10 μg/ml of antibody, which are listed in Table 18.

TABLE 18
CDC activity/cell staining ratios.

| | | Staining with | | |
|---|---|---|---|---|
| | | propidium | Staining of | |
| | | iodide (CDC) | membrane Gb3 | Ratios |
| HMEC-1 cells | 3E2 | 51.3 ± 9.2% | 75.0 ± 11.3% | 68.2 ± 6.2% |
| | 1A4 | 65.2 ± 13.2% | 92.7 ± 5.9% | 69.9 ± 10.2% |
| Raji cells | 3E2 | 71.8 ± 5.5% | 91.6 ± 5.3% | 78.7 ± 8.2% |
| | 1A4 | 82.9 ± 7.7% | 99.3 ± 0.2%. | 83.4 ± 7.8% |

For HMEC-1 cells, the ratio is evaluated at 68.2±6.2% for antibody 3E2 and 69.9±10.2% for antibody 1A4. For Raji cells, the ratio is evaluated at 78.7±8.2% for antibody 3E2 and 83.4±7.8% for antibody 1A4. Thus, both antibodies have comparable CDC activities. Antibody 1A4 has greater CDC activity values than antibody 3E2 because its affinity for Gb3 is greater.

5.2 Conclusion

Antibodies act mainly in three ways, which can be cumulative. By direct binding, they can induce cytostatic activities (like blocking the cell cycle) or cytotoxic activities (like apoptosis and necrosis). By CDC, they can induce the activation of complement proteins by binding protein complex C1q on the Fc region of antibodies bound to their target, thus forming the membrane attack complex and lysing the cell. By ADCC, the FcγR receptors bind the Fc region of antibodies bound to their target, leading to lysis or phagocytosis induced by an effector cell of the immune system.

The various biological tests implemented show that antibody 3E2 initially has a cytostatic activity independent of complement. MTT shows an inhibition of cellular viability of HMEC-1 cells equal to 14.6±1.6% for 20 μg/ml of antibody, by 24 h (FIG. 16). This result is confirmed by a video-kinetic study of HMEC-1 cells. When the cells are incubated with 20 μg/ml of antibody, a reduction in the total number of cells near the inhibition value that is obtained by MTT is observed (FIG. 18). There are fewer dividing cells and their division time is extended overall. At 72 h, the inhibition of HMEC-1 cellular viability is 22.2±9.1%. This value is close to the maximum value that is obtained at 24 h with 40 μg/ml of antibody 3E2 (16.3±3.1%; FIG. 16). This can be explained by the presence of HMEC-1 cells weakly expressing Gb3, which are then less subject to inhibition of cellular viability induced by antibody 3E2 and by the very rapid growth of these cells transformed by SV40 virus. This is why we reduced our video-kinetic study to the first 24 h, because beyond this, HMEC-1 cells invade the analysis field very quickly and it is no longer possible to count them.

In the presence of antibody 3E2, an increase in the frequency of mitotic aberrations is also observed, associated with the presence of cells blocked in division for several hours and which finally burst, as well as the presence of cells that are not able to enter into division and which alternate repeated cycles of cellular attachment and detachment. Such mitotic aberrations can be attributed to death mechanisms involving adhesion, such as death by anoikis, as already demonstrated for antibodies targeting ganglioside GD₂ in small cell lung cancers. Anoikis is a death phenomenon that was demonstrated for the first time in 1994 and which is characterized by the loss of contact of cells with their extracellular matrix. It has been shown that anti-GD₂ antibody induces apoptosis via the ubiquitous cytoplasmic protein FAK (focal adhesion kinase) found within the adhesion complex and which can be activated by integrins as well as by various growth factors, cytokines or hormones. As for Gb3, it has already been shown for microvascular endothelial cells derived from patients with Fabry disease, that Gb3 present in large quantities was associated with an increased expression of molecules involved in cell adhesion such as ICAM-1, VCAM-1 and E-selectin proteins. Zemunic et al. (2004) compared the distribution of Gb3 with that of E-selectin in HUVEC cells stimulated with TNF-α. The activated cells were characterized by an increase in the expression of selectin E associated with an increase in the expression of Gb3. These results suggest that Gb3 could have a potential role in the mechanisms of adhesion in the endothelium (Zemunik et al. 2004).

Furthermore, we have shown by immunocytochemistry in proliferating cells that there is a polarization of membrane Gb3 at the contact area of two interacting HMEC-1 cells (data not shown). When the cell is isolated, the membrane distribution of Gb3 is fairly homogeneous (data not shown). It has recently been described that the increase of the glycosphingolipid content in tumor tissue, such as Gb3, whose content is 2 to 3 times higher in epithelial carcinomas, could be reflected by morphological changes at the membrane microdomains (Marques Filho et al. 2006). These changes would be responsible for a better interaction of cells among themselves and cells with their extracellular matrix, which would increase their ability to infiltrate and their metastatic potential (Marques Filho et al. 2006). The migration of cells requires setting up a finely regulated system of cytoskeleton and adhesion complex organization. In endothelial cells, treatment with IFN-γ increases the relative proportion of intracellular Gb4 that is associated with the cell cytoskeleton. The specific modulation of glycosphingolipids by IFN-γ suggests that they play a role in the adhesion mechanisms of activated endothelial cells. Notably, the most abundant glycolipids of HUVEC cells, Gb4 and GM₃ are located at the surface and intracellularly, where they are associated with vimentin intermediate filaments of the cytoskeleton which could play a role in the transport of glycosphingolipids. It was then shown that after binding Gb3, verotoxin is internalized to the rough endoplasmic reticulum and the nuclear envelope, suggesting that Gb3 may play a role in cell growth since its expression varies depending on the cell cycle, and that the cells in the G1/S phase are more sensitive to binding of the toxin, while cells in the stationary phase are resistant. It seems that Gb3 expression variations are associated with the cell cycle, cell migration and cell adhesion.

The high membrane expression of an antigen, especially if it is not subject to internalization phenomena, can yield a high antigen-antibody complex density at the membrane, promoting the recruitment of immunity effectors, especially complement. The classical pathway is activated by the antigen-antibody complex and among the immunoglobulins, IgM is an effective activator of complement-dependent cytoxicity. Antibody 3E2 is capable of activating CDC from 0.5 μg/ml in HMEC-1 cells (20.6±3.2% of cells lysed) which have 1.7 10⁶ Gb3 sites and from 0.1 μg/ml in Raji cells (15.7±5.2%) which have 2.0 10⁶ sites. For antibody 1A4, CDC activity is observed from 0.1 μg/ml on HMEC-1 cells (22.2±6.9%) and Raji cells (25.8±3.9%). The two antibodies are thus able to activate the complement pathway for small antibody concentrations. This complement-dependent activity allows us to consider future preclinical studies in mice.

### Example 6

### Anti-Gb3 Antibodies According to the Invention

The seven monoclonal antibodies selected (3E2, 14C11, 15C11, 22F6, 25C10, 11E10, 16G8) were obtained from a single somatic hybridization and a single cloning. They are all of isotype IgM and all have a κ type light chain. To analyze the mechanisms of the immune response with Gb3 and their structure-function relationship, we sequenced the V_{H} and V_{L} gene segments of antibodies directed specifically against glycolipid Gb3. The problem was to determine what this specificity was due to: either the restricted expression of the VDJ gene repertoire, or the presence of somatic mutations.

Genes Used for the Heavy Chain Variable Regions of Anti-Gb3 Antibodies.

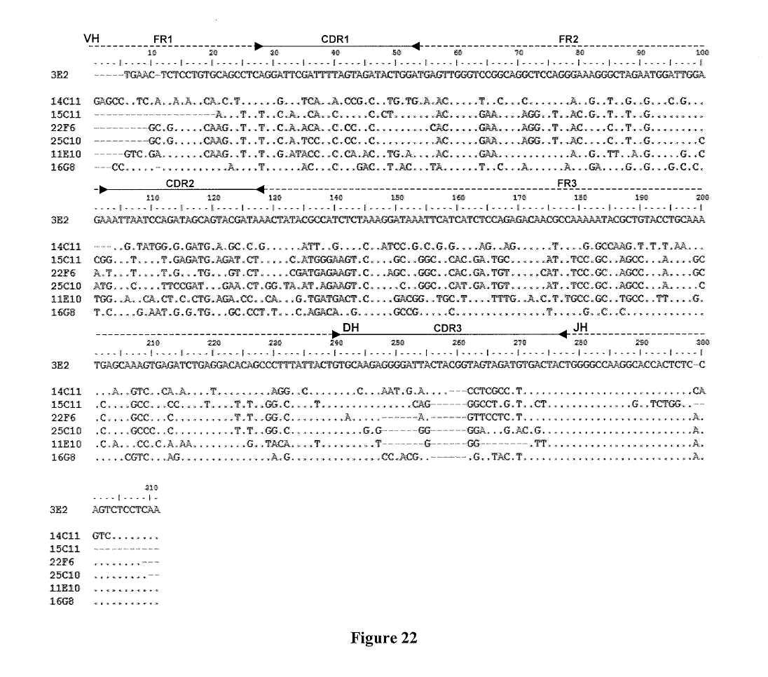

The nucleotide sequences of the heavy chains of the seven anti-Gb3 antibodies are shown in FIG. 22.

The genes used for the heavy chain variable regions are listed in Table 19 below.

TABLE 19
Genes used for the heavy chain variable regions.

| | | | J gene | | | | | AA sequence |
|---|---|---|---|---|---|---|---|---|
| Sequence | V gene and | | and its | | D gene and | Length of | | of the |
| name | its allele | Homology | allele | Homology | its allele | CDR | FR | junction |
| 15C11_VH2A | IGHV1- | 96.83% | IGHJ3*01 | 100% | IGHD3-3*01 | 8.8.10 | [2.17.38.7] | CARGDRAW |
| | 82*01 | (214/221 nt) | | (35/35 nt) | | | | FAYW |
| 22F6_VH2B | IGHV1S22 | 98.71% | IGHJ2*01 | 93.33% | IGHD5-1*01 | 8.8.8 | [6.17.38.10] | CTRKFLFDYW |
| | *01 | (229/232 nt) | | (42/45 nt) | | | | |
| 25C10_VH2B | IGHV1- | 93.09% | IGHJ2*01 | 84.78% | IGHD3-3*01 | 8.8.8 | [6.17.38.10] | CAGGDRYG |
| | 74*01.or | (216/232 nt) | | (39/46 nt) | | | | YW |
| | IGHV1- | | | | | | | |
| | 74*04 | | | | | | | |
| 14C11_VH1A | IGHV2-6- | 100% | IGHJ2*01 | 81.65% | IGHD2-1*01 | 8.7.11 | [9.17.38.11] | CARNGNYL |
| | 7*01. or | (238/238 nt) | | (39/48 nt) | | | | AFDYW |
| | IGHV2-6- | | | | | | | |
| | 7*02 | | | | | | | |
| 3E2_VH1A | IGHV4- | 99.15% | IGHJ2*01 | 65.96% | IGHD1-1*01 | 8.8.12 | [7.17.38.11] | CARGDYYG |
| | 1*02 | (233/235 nt) | | (31/47 nt) | | | | SRCDYW |
| 16G8_VH3A | IGHV5- | 98.73% | IGHJ2*01 | 93.75% | IGHD2-11*01 | 8.8.10 | [8.17.38.11] | CARPTYGYF |
| | 12*02 | (234/237 nt) | | (45/48 nt) | | | | DYW |
| 11E10_VH2C | IGHV9-3- | 98.73% | IGHJ2*01 | 84.09% | IGHD2-3*01 | 8.8.5 | [7.17.38.11] | CASGVYW |
| | 1*01 | (233/236 nt) | | (37/44 nt) | | | | |

According to the table above, the seven specific antibodies of glycolipid Gb3 each use a different V gene with a degree of homology to the germ-line configuration greater than 98% except for the case of mAb 15C11 and 25C10, which are, respectively, distinguished by 96.8 and 93.1% homology. For mAb 14C11 a homology of the V gene identical to allele IGHV2-6-7*01 or 02 will be noted. Moreover, except for mAb 15C11, which has 100% homology with allele IGHJ3*01, the six other mAb all use the same J gene (IGHJ2*01) which combines with a D gene, different products of VH sequences, different for each hybridoma. The length of the hypervariable region CDR3H is comprised between 5 and 12 amino acids.

Genes Used for the Light Chain Variable Regions of Anti-Gb3 Antibodies.

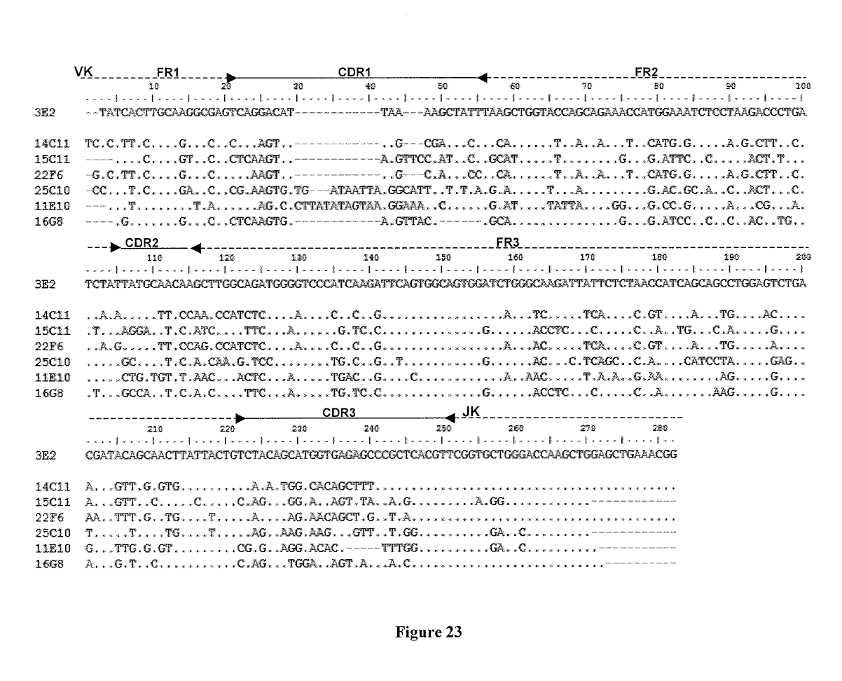

The nucleotide sequences of the heavy chains of the seven anti-Gb3 antibodies are shown in FIG. 23.

The genes used for the light chain variable regions are listed in Table 20 below.

TABLE 20
Genes used for the light chain variable regions.

| | | | J gene | | | | | | AA |
|---|---|---|---|---|---|---|---|---|---|
| Sequence | V gene and | | and its | | Length of | | | | sequence of |
| name | its allele | Homology | allele | Homology | CDR 1 | CDR 2 | CDR 3 | FR | the junction |
| 11E10_VKA | IGKV1- | 100% | IGKJ1*01 | 100% | 11 | 3 | X | [6.17.36.7] | CVQGTH## |
| | 133*01 | (234/234 nt) | | (28/28 nt) | | | | | TF |
| 25C10_VKA | IGKV3- | 100% | IGKJ1*01 | 100% | 10 | 3 | 9 | [6.17.36.7] | CQQSKEVP |
| | 2*01 | (233/233 nt) | | (26/26 nt) | | | | | RTF |
| 16G8_VKF | IGKV4- | 99.53% | IGKJ5*01 | 100% | 5 | 3 | 9 | [5.17.36.7] | CQQWSSNP |
| | 72*01 | (214/215 nt) | | (25/25 nt) | | | | | PTF |
| 15C11_VKF | IGKV4- | 100% | IGKJ2*01 | 100% | 7 | 3 | 9 | [5.17.36.8] | CQQGSSIP |
| | 91*01 | (221/221 nt) | | (23/23 nt) | | | | | RTF |
| 14C11_VKA | IGKV5- | 100% | IGKJ5*01 | 100% | 6 | 3 | 9 | [7.17.36.10] | CQNGHSFP |
| | 39*01 | (222/222 nt) | | (38/38 nt) | | | | | LTF |
| 22F6_VKF | IGKV5- | 99.55% | IGKJ5*01 | 100% | 6 | 3 | 9 | [6.17.36.10] | CQQSNSWP |
| | 45*01 | (220/221 nt) | | (35/35 nt) | | | | | HTF |
| 3E2_VKF | IGKV14- | 90.91% | IGKJ5*01 | 100% | 6 | 3 | 9 | [6.17.36.10] | CLQHGESP |
| | 111*01 | (200/220 nt) | | (38/38 nt) | | | | | LTF |

The alignment of nucleotide sequences coding for the variable region Vκ of the seven monoclonal antibodies first shows a use of V genes that are all different but are almost all in germ-line configuration, with the exception of mAb 3E2 that we selected as having the most affinity of all. Its specificity is most likely associated with the variability of the J segment of VH (65.9%) combined with that of the V gene segment of Vκ (90.9%). It is also interesting to note that mAb 3E2 is encoded by the same J gene (IGKJ5*01) as mAb 14C11 and 22F6 with the same degree of homology but with a different expression of amino acids at the junction from one hybridoma to another.

The seven anti-Gb3 antibodies were generated during a single somatic hybridization and the similarities observed in the heavy and light chains suggest that the antibodies are related by cloning. Our results indicate that a variety of V_{H} and V_{L} chains can encode anti-Gb3 antibodies and that some are subject to a maturation process marked by several somatic mutations. All the nucleotide data collected for the expression of the seven mAb specific for Gb3 confirm, after cloning of the hybridomas, the existence of distinct combinations of VDJ and VJ genes for the expression of mAb, apparently without any restriction.

It could actually be very interesting to follow the measurement of monoclonal affinity and the state of the respective sequences at the same time to understand the nature of the interactions of mAbs and glycolipid Gb3. By directed mutagenesis, it could be very easy to define the critical amino acids of the variable regions and more particularly the hypervariable regions that typically confer antibody specificity.

We are currently in the process of evaluating the affinities of each of the seven antibodies by the Scatchard technique in order to demonstrate more precisely the relationships existing between the nucleotide structure of the antibody and its affinity.

Still, sequencing of the V_{H} and V_{L} regions of mAb is essential to design and optimize chimeric or humanized antibodies for therapeutic purposes in humans if human antibodies cannot be obtained. This approach requires at least three-dimensional modeling of the V_{H} and V_{L} variable regions of the antibody adapted to the specific recognition of Gb3 to substitute the hypervariable regions of a human antibody with those of mAb 3E2 that we have defined as being the leader of the seven antibodies that we characterized with regard to glycolipid Gb3.

### Example 7

### In-Vivo Confirmation of the Antiangiogenic Properties of Antibody 3E2 in Two Tumor Models

The therapeutic potential of antibody 3E2 in the treatment of solid tumors has been confirmed in vivo in two tumor models based on the mouse neuroblastoma line NXS2 and compared to that of an anti-GD2 antibody.

These two models based on mouse neuroblastoma NXS2 were chosen for the following reasons:
Insofar as NXS2 cells do not express Gb3 (see Example 3 and the results below), these models allow precisely evaluating the in vivo effects of antibody 3E2 on tumor vessels (i.e., its antiangiogenic properties), and not a combination of effects on tumor vessels and on the tumor cells themselves, as would be the case if the tumor cells chosen also expressed Gb3.
In this model, there is a reference antibody treatment corresponding to anti-GD2 antibodies, since GD2 is another glycolipid that, unlike Gb3, is expressed by NXS2 tumor cells and not expressed by blood vessel cells.

These two models therefore permit assessing the in-vivo antiangiogenic properties of antibody 3E2 and comparing its efficacy to that of an antibody considered to be the standard treatment for the chosen disease.

7.1 Materials and Methods

NXS2 Tumors Established Subcutaneously

Female AJ mice were inoculated subcutaneously in the right flank with 10⁶ living NXS2 cells (neuroblastoma line) (viability >95%) resuspended in 150 μL of PBS. Tumor progression was measured every day. The long (D) and short (d) diameters of each tumor were measured with a caliper. Tumor volume V was calculated by using the following formula: V=0.5×D×d². When the tumors reached a volume V of 100-150 mm³, the mice received a treatment with antibody 3E2 directed against the upper band of the Gb3 doublet expressed by HMEC-1 cells, anti-GD2 antibody 14G2a, an IgM isotype control antibody or PBS alone. The treatment consisted of a single injection in 150 μL of PBS. Tumor progression was measured as previously and expressed as a percentage of increase with regard to the tumor volume at the time of injection.

NXS2 Metastatic Tumor Model

Mouse

Male A/J mice (age 6-8 weeks) were obtained from Harlan Sprague-Dawley (Sulzfeld, Germany). Animal studies were conducted in compliance with Directive 86/609/EEC

Tumor Model

Experimental hepatic metastases were induced by injection in the tail vein at day 0 of 2.5 10⁵ NXS2 tumor cells (viability >95%) in 200 μL of PBS. The mice were treated with intravenous injections of monoclonal antibody at days 1, 2, 7 and 8 for antibody 3E2 (IgM, 200 μg in 150 μL of PBS), or at days 1 and 2 for antibody 14G2a (IgG, 100 μg in 100 μL) or for an IgG isotype control antibody (100 μg in 100 respectively. The molar quantities of antibody for each injection are similar for all the antibodies, the larger mass used for antibody 3E2 (IgM) with regard to other antibodies (IgG) being due to the higher molecular weight of IgM compared to IgG. Likewise, the new injections of antibody 3E2 at days 7 and 8 are made necessary by the half-life of IgM in vivo (around 1 week), which is not the case for the other antibodies of isotype IgG, since IgG has a half-life in vivo of around 3 weeks.

Control mice only received PBS. The animals were sacrificed at day 28, their liver was weighed and the number of metastases evaluated.

Histology and Immunohistochemical Staining

Frozen tumors were sliced and fixed in cold acetone for 10 minutes. The samples were stained for 90 minutes with the following monoclonal antibodies: a rat antibody directed against mouse CD31 (1:50; Millipore, Molsheim, France) and a biotinylated antibody 3E2, 14G2a (anti-GD2) or its isotype control (Beckmann Coulter, Fullerton, Calif., USA) (40 μg/ml). Antibody 3E2 and the isotype control were biotinylated with an EZ-Link Sulfo-NHS-LC-Biotinylation kit (Thermo Scientific, Courtaboeuf, France). Staining was revealed by 90 minutes of incubation with a goat anti-rat antibody conjugated with AF 488 (1:400; Invitrogen) for CD31 and with streptavidin conjugated with AF 568 (1:200; Invitrogen) for antibody 3E2 or its isotype control. The samples were finally coated with a ProLong Gold Antifade reagent with DAPI. Organs from healthy animals were also stained with antibody 3E2 or its isotype control by using the same protocol. The staining was observed under a confocal fluorescence microscope (Nikon, Champigny sur Marne, France). Muscle sections were used as a normal tissue control.

7.2 Results

NXS2 Tumors Established Subcutaneously

The tumor volumes were comprised between 120 and 130 mm³ and did not significantly differ during the treatment injection (antibodies 3E2, 14G2a, control IgM, or PBS).

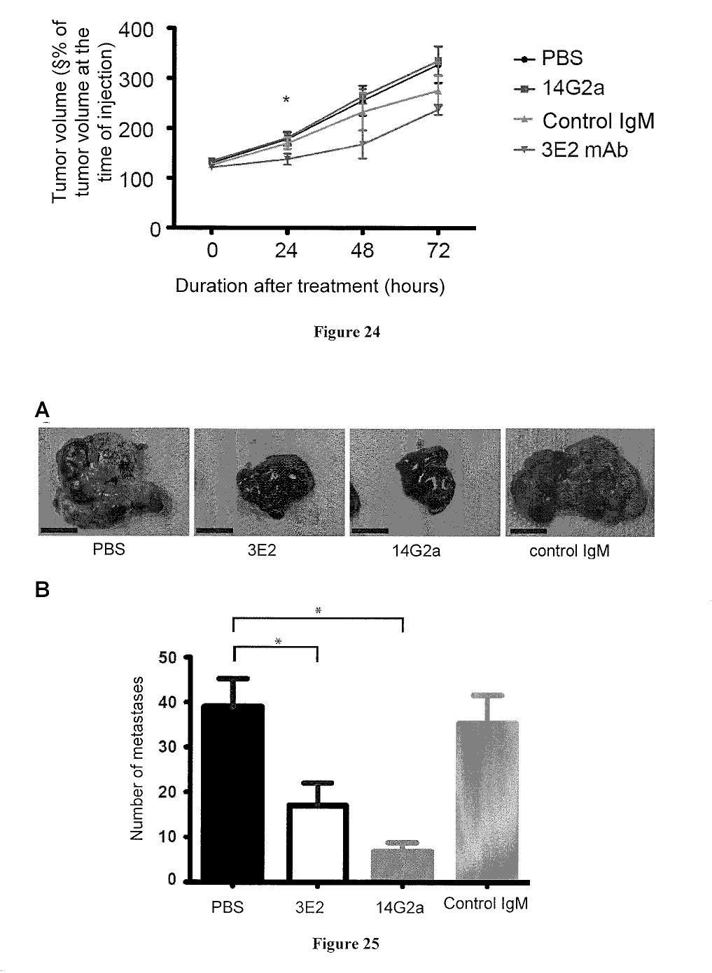

The change in the tumor volume after treatment is shown in FIG. 24. At 24 hours after treatment, a single injection with antibody 3E2 significantly reduced tumor volume at 24 h compared to treatment with PBS alone (tumor volume of 178.3±13.19 mm³ with PBS (n=11) vs. 137.9±11.08 mm³ for antibody 3E2 (n=8)). This is not the case for the other treatments (antibody 14G2a or control IgM).

At 48 and 72 hours, treatment with antibody 3E2 led to a smaller tumor volume, although the difference is not statistically significant. This is probably explained by the fact that too few animals were tested at 48 and 72 hours after treatment.

NXS2 Metastatic Tumor Model

The efficacy of antitumor antibody 3E2 was then determined in an experimental hepatic metastasis model of mouse neuroblastoma NXS2 developed by Lode et al.

It was first verified that antibody 3E2 specifically targets blood vessels within the tumor mass by histology and immunohistochemical staining. The results are presented in Table 21 below and show that antibody 3E2 generates an intense staining co-localized with that of the anti-CD31 antibody (cell blood vessel marker), and no staining in the tumor mass. The muscle sections did not show any Gb3 staining, only endothelial cells within tumor vessels were stained by antibody 3E2. In return, NXS2 tumor cells show a high level of GD2.

TABLE 21
Distribution of Gb3 within the tumor mass of NXS2
metastases. The distribution of GD2, Gb3 and CD31
within the tumor mass of untreated mouse NXS2 metastases
was detected by immunohistochemistry.

| | | Tumor | |
|---|---|---|---|
| Antigen | Neuroblastoma cells | endothelial cells | Muscle |
| GD2 | + | − | − |
| CD31 | − | + | − |
| Gb3 | − | + | − |
| Control antibody | − | − | − |

−, no staining; +, positive staining. Muscle sections were used as a normal tissue control.

To evaluate the therapeutic benefit of monoclonal antibody 3E2 injections and compare it to immunotherapy with anti-GD2 monoclonal antibody 14G2a, the number of NXS2 liver metastases and the weight of the liver were evaluated after these different treatments (FIG. 25).

Treatment of mice with antibodies 3E2 and 14G2a proved very effective for reducing neuroblastoma hepatic metastases, as indicated by the reduction of liver weight from 39±16.23 g (mice treated with PBS) to 17±5.03 g (mice treated with antibody 3E2) and 6.75±12.06 g (mice treated with antibody 14G2a) (p>0.05). These last two values are not significantly different from those found in healthy control animals (p>0.1). The effect of treatment with monoclonal antibody 3E2 is not significantly different from treatment with monoclonal antibody 14G2a (p>0.5).

These data also confirm the specificity of treatment with antibody 3E2, since treatment with an isotype control antibody is completely ineffective.

Because antibody 3E2 is an IgM, whose distribution is limited to the intravascular compartment and because it recognizes blood vessels in solid tumors but not in healthy tissues, the results suggest that its therapeutic effects are linked to its action on these blood vessels.

7.3 Conclusion

These results show for the first time that passive immunotherapy with anti-Gb3 antibody 3E2 is effective to suppress the growth of tumor metastases in a relevant syngeneic neuroblastoma model in mice which is similar to the human disease (Lode et al.). This tumor model expresses disialoganglioside GD2, a well-known antigen associated with neuroblastoma (Lode et al.), but not Gb3, as demonstrated in vitro and in vivo.

Likewise, the injection of antibody 3E2 is also effective to inhibit subcutaneous NXS2 neuroblastoma tumors in A/J mice.

The distribution of Gb3 in NXS2 metastases, shown by staining with antibody 3E2 and validated by CD31 staining of tissue sections, is clearly limited to the endothelial cells of tumor compartment blood vessels. Consequently, the results obtained show that the antivascular activity of anti-Gb3 antibody 3E2 is sufficient to inhibit tumor growth in vivo. The results obtained also show that the anti-tumor efficacy of anti-Gb3 antibody 3E2 is comparable to that for anti-GD2 antibody 14G2a, which directly targets NXS2 neuroblastoma cells and which has been the subject of clinical evaluations with positive results (Murray et al.).

### REFERENCES

Ades, et al (1992). J Invest Dermatol 99, 683-690
Almagro et al. Frontiers in Bioscience 13, 1619-1633, Jan. 1, 2008,
Askari et al. 2007. Virchows Arch 451, 823-834
Bast D J et al. Infect Immun. 1997 June; 65(6): 2019-28,
Bonnaud et al. 2007. Cancer Res 67, 1803-1811
Bordron A et al. J Clin Invest. 1998 May 15; 101(10): 2029-35,
Brochet, X. et al. Nucl. Acids Res. 36, W503-508 (2008)
Chark D et al. Eur J Biochem. 2004 January; 271(2): 405-17,
Chomczynski and Sacchi, (1987). Anal Biochem 162, 156-159.
Clackson et al. 1991, Nature 352, 624-628
EP1176195A1,
EP1331266A1,
Falguieres et al. 2008. Mol Cancer Ther 7, 2498-2508
Folch, J., et al. (1951). J Biol Chem 191, 833-841
Gariepy, 2001. Crit Rev Oncol Hematol 39, 99-106
Gaugler et al. 2007. Radiat Res 167, 185-193
Hakomori and Kannagi, 1983. J Natl Cancer Inst 71, 231-251
Heath-Engel H M et al. Angiogenesis. 2003; 6(2): 129-41,
Janssen K P et al. Cancer Res. 2006 Jul. 15; 66(14): 7230-6,
Johansson et al. 2009, BMC Cancer 9, 67
Jones et al. Nature, 321: 522-525, 1986;
Kabat et al. Sequences of proteins of immunological interest, 5^{th} Ed., U.S. Department of Health and Human Services, NIH, 1991, and later editions,
Kanda et al. 2004. J Neurosci Res 78, 141-150
Khodarev et al. 2003. J Cell Sci 116, 1013-1022
Kotani et al. 1994. Arch Biochem Biophys 310, 89-96
LaCasse et al. 1999. Blood 94, 2901-2910
Ladisch and Li. Methods Enzymol. 2000; 312: 135-45,
Lefranc and Lefranc, 1997 Ingénierie des anticorps banques combinatoires [Engineering antibody combinatorial libraries]. eds. Lefranc, M. P. and Lefranc, G. Les éditions INSERM, Paris
Lefranc, M.-P. et al. Dev. Comp. Immunol., 27, 55-77 (2003),
Lingwood et al. Bioscience Reports, Vol. 19, No. 5, 1999,
Lode et al. Proc Natl Acad Sci USA. 1999 Feb. 16; 96(4):1591-6,
Mangeney et al. 1993. Cancer Res 53, 5314-5319
Marques Filho et al. 2006. Otorhinolaryngol 72, 25-30
Morita, Y., et al. (2000). Nat Med 6, 1109-1114,
Murray J L, et al. J Clin Oncol. 1994; 12:184-93,
Müthing et al. 1998. Carbohydr Res 307, 147-157
Müthing et al. 1999. Glycobiology 9, 459-468
Nikolayenko I V et al. Ukrainica Bioorganica Acta 2 (2005) 3-11
Obrig T G et al. Infect Immun. 1988 September; 56(9): 2373-8,
Obrig et al. 1993. J Biol Chem 268, 15484-15488
Riechmann et al. Nature, 332: 323-327, 1988,
Sanger et al. 1977, Proc Natl Acad Sci USA 74, 5463-5467
Shields et al. Journal of Biological Chemistry. Vol. 276, No. 9, Issue of March 2, pp. 6591-6604, 2001,
Taga et al. 1997. Blood 90, 2757-2767
Tétaud C et al. J Biol Chem. 2003 Nov. 14; 278(46): 45200-8,
U.S. Pat. No. 7,432,357B2,
Verhoeyn et al. BioEssays, 8: 74, 1988,
Verhoeyen et al. Science, 239: 1534-1536, 1988;
Viel et al. Mol. Imaging. 2008 November-December; 7(6):239-47;
Wiels et al. 1981. Proc Natl Acad Sci USA 78, 6485-6488,
WO98/51326,
WO01/77181,
Zemunik et al. 2004. Biochemistry (Mosc) 69, 513-519

1-21. (canceled)

22. An antibody directed against the membrane glycosphingolipid globotriaosylceramide (Gb3), or a functional fragment or a derivative thereof, characterized in that it has at least one complementarity determining region (CDR) with an amino acid sequence selected from SEQ ID NO: 1 to 42, or with an amino acid sequence having at least 80% identity with one of SEQ ID NO: 1 to 42.

23. The antibody, functional fragment or derivative thereof according to claim 22, which has at least one heavy chain comprising at least one complementarity determining region (CDR) with an amino acid sequence selected from SEQ ID NO: Ito 3, 7 to 9, 13 to 15, 19 to 21, to 27, 31 to 33, and 37 to 39, or with an amino acid sequence having at least 80%-identity with one of SEQ ID NO: 1 to 3, 7 to 9, 13 to 15, 19 to 21, 25 to 27, 31 to 33, and 37 to 39.

24. The antibody, functional fragment or derivative thereof according to claim 22, which has a light chain comprising at least one complementarity determining region (CDR) with an amino acid sequence selected from SEQ ID NO: 4 to 6, 10 to 12, 16 to 18, 22 to 24, 28 to 30, 34 to 36, and 40 to 42, or with an amino acid sequence having at least 80% identity with one of SEQ ID NO: 4 to 6, 10 to 12, 16 to 18, 22 to 24, 28 to 30, 34 to 36, and 40 to 42.

25. The antibody, functional fragment or derivative thereof according to claim 22, which has a heavy chain comprising three CDR-H (heavy chain CDR) with the following amino acid sequences, or sequences having at least 80% identity with the following sequences:
a) CDR1-H-3E2: SEQ ID NO: 1, CDR2-H-3E2: SEQ ID NO: 2, CDR3-H-3E2: SEQ ID NO: 3,
b) CDR1-H-14C11: SEQ ID NO: 7, CDR2-H-14C11: SEQ ID NO: 8, CDR3-H-14C11: SEQ ID NO: 9,
c) CDR1-H-15C11: SEQ ID NO: 13, CDR2-H-15C11: SEQ ID NO: 14, CDR3-H-15C11: SEQ ID NO: 15,
d) CDR1-H-22F6: SEQ ID NO: 19, CDR2-H-22F6: SEQ ID NO: 20, CDR3-H-22F6: SEQ ID NO: 21,
e) CDR1-H-25C10: SEQ ID NO: 25, CDR2-H-25C10: SEQ ID NO: 26, CDR3-H-25C10: SEQ ID NO: 27,
f) CDR1-H-11E10: SEQ ID NO: 31, CDR2-H-11E10: SEQ ID NO: 32, CDR3-H-11E10: SEQ ID NO: 33, or
g) CDR1-H-16G8: SEQ ID NO: 37, CDR2-H-16G8: SEQ ID NO: 38, CDR3-H-16G8: SEQ ID NO: 39.

26. The antibody, functional fragment or derivative thereof according to claim 22, which has a light chain comprising three CDR-L (light chain CDR) with the following amino acid sequences, or sequences having at least 80% identity with the following sequences:
a) CDR1-L-3E2: SEQ ID NO: 4, CDR2-L-3E2: SEQ ID NO: 5, CDR3-L-3E2: SEQ ID NO: 6,
b) CDR1-L-14C11: SEQ ID NO: 10, CDR2-L-14C11: SEQ ID NO: 11, CDR3-L-14C11: SEQ ID NO: 12,
c) CDR1-L-15C11: SEQ ID NO: 16, CDR2-L-15C11: SEQ ID NO: 17, CDR3-L-15C11: SEQ ID NO: 18,
d) CDR1-L-22F6: SEQ ID NO: 22, CDR2-L-22F6: SEQ ID NO: 23, CDR3-L-22F6: SEQ ID NO: 24,
e) CDR1-L-25C10: SEQ ID NO: 28, CDR2-L-25C10: SEQ ID NO: 29, CDR3-L-25C10: SEQ ID NO: 30,
f) CDR1-L-11E10: SEQ ID NO: 34, CDR2-L-11E10: SEQ ID NO: 35, CDR3-L-11E10: SEQ ID NO: 36, or
g) CDR1-L-16G8: SEQ ID NO: 40, CDR2-L-16G8: SEQ ID NO: 41, CDR3-L-16G8: SEQ ID NO: 42.

27. The antibody, functional fragment or derivative thereof according to claim 22, which has a heavy chain comprising a variable region with a sequence selected from SEQ ID NO: 43 to 49, or with a sequence having at least 80% identity with one of SEQ ID NO: 43 to 49.

28. The antibody, functional fragment or derivative thereof according to claim 22, which has a light chain comprising a variable region with a sequence selected from SEQ ID NO: 50 to 56, or with a sequence having at least 80% identity with one of SEQ ID NO: 50 to 56.

29. The antibody, functional fragment or derivative thereof according to claim 22, which has heavy and light chains whose variable regions have the following amino acid sequences, or sequences having at least 80% identity with the following sequences:
a) Antibody 3E2: heavy chain: SEQ ID NO: 43, light chain: SEQ ID NO: 50,
b) Antibody 14C11: heavy chain: SEQ ID NO: 44, light chain: SEQ ID NO: 51,
c) Antibody 15C11: heavy chain: SEQ ID NO: 45, light chain: SEQ ID NO: 52,
d) Antibody 22F6: heavy chain: SEQ ID NO: 46, light chain: SEQ ID NO: 53,
e) Antibody 25C10: heavy chain: SEQ ID NO: 47, light chain: SEQ ID NO: 54,
f) Antibody 11E10: heavy chain: SEQ ID NO: 48, light chain: SEQ ID NO: 55, and
g) Antibody 16G8: heavy chain: SEQ ID NO: 49, light chain: SEQ ID NO: 56.

30. The antibody, functional fragment or derivative thereof according to claim 29, which has heavy and light chains whose variable regions have the following amino acid sequences, or sequences having at least 80%-identity with the following sequences:
a) Antibody 3E2: heavy chain: SEQ ID NO: 43, light chain: SEQ ID NO: 50, and
b) Antibody 22F6: heavy chain: SEQ ID NO: 46, light chain: SEQ ID NO: 53.

31. The antibody, functional fragment or derivative thereof according to claim 22, which is of isotype IgG or IgM.

32. The antibody, functional fragment or derivative thereof according to claim 22, which is a chimeric or humanized antibody.

33. The functional fragment of antibody according to claim 22, which is selected from fragments Fv, ScFv, Fab, F(ab′)2, Fab′, scFv-Fc or diabodies.

34. The antibody, functional fragment or derivative thereof according to claim 22, wherein said antibody, functional fragment or derivative thereof is not bound to a cytotoxic molecule.

35. A nucleic acid encoding the antibody according to claim 22.

36. A vector comprising a nucleic acid according to claim 35.

37. A host cell comprising the nucleic acid according to claim 35.

38. A method for treating a disease associated with angiogenesis in a subject in need thereof, comprising the administration of an effective quantity of an antibody directed against the membrane glycosphingolipid Gb3 and not coupled to a therapeutic molecule.

39. The method of claim 38, wherein said disease associated with angiogenesis is selected from solid tumors, psoriasis, angiomas, proliferative eye diseases, and autoimmune diseases.

40. A method for treating a disease associated with angiogenesis in a subject in need thereof, comprising the administration of an effective quantity of an antibody directed against the membrane glycosphingolipid Gb3 and not coupled to a therapeutic molecule, wherein said antibody directed against the membrane glycosphingolipid Gb3 and not coupled to a therapeutic molecule is the antibody according to claim 35.

41. The method according to claim 38, wherein said antibody directed against the membrane glycosphingolipid Gb3 and not coupled to a therapeutic molecule recognizes the upper band but not the lower band of the Gb3 doublet expressed by HMEC-1 cells.

42. A method to inhibit angiogenesis in a subject in need thereof, comprising the administration of an effective quantity of an antibody directed against the membrane glycosphingolipid Gb3 and not coupled to a therapeutic molecule.

43. A host cell comprising the vector according to claim 36.
